(19) United States
(12) Patent Application Publication
(10) Pub. No.: US 2004/0014209 A1
(43) Pub. Date: Jan. 22, 2004

(54) Compositions and methods for modulating cell differentiation

(76) Inventors: Andrew B. Lassar, Newton Center, MA (US); Mark Mercola, Del Mar, CA (US); Ruchika Gupta, San Diego, CA (US); Martha Marvin, Brookline, MA (US); Valerie Schneider, Philadelphia, PA (US); Eldad Tzahor, Brookline, MA (US); Barbara Brott, Boston, MA (US); Sergei Sokol, Boston, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/351,275

(22) Filed: Jan. 23, 2003

Related U.S. Application Data
(60) Provisional application No. 60/351,126, filed on Jan. 23, 2002.
(60) Provisional application No. 60/352,456, filed on Jan. 28, 2002.
(60) Provisional application No. 60/352,665, filed on Jan. 29, 2002.

(51) Int. Cl. C12N 15/85; C12N 5/08

(52) U.S. Cl. 435/366; 435/455

(57) ABSTRACT

The present invention relates to compositions and methods for stimulating differentiation of stem cells into cardiac cells. The methods of the invention involve contacting a population cells comprising stem cells with at least one Wnt antagonist, such as a polypeptide or polypeptide fragment. In certain embodiments, the methods of the invention involve Dkk proteins or fragments, homologs, derivatives, variants, or peptidomimetics thereof.

### RELATED APPLICATION INFORMATION

This application claims the benefit of priority to Provisional Patent Application Nos. 60/351,126, filed Jan. 23, 2002, 60/352,456, filed Jan. 28, 2002, and 60/352,665, filed Jan. 29, 2002, which applications are hereby incorporated by reference in their entirety.

### GOVERNMENT SUPPORT

This invention was made with government support by the National Institutes of Health under award numbers HD31247, HL59502, PO 50 HL61036 and P050 HL61036-01. The government has certain rights in the invention.

### BACKGROUND OF THE INVENTION

The heart and the derivatives of the blood islands are the first mesodermal tissues to differentiate after gastrulation in amniote embryos. Cells that migrate anterior and lateral to the primitive streak in early gastrulation contribute to heart tissue, whereas cells that move into the posterior lateral plate form the extraembryonic blood islands (Rosenquist and DeHaan, 1966. Carnegie Inst. Washington Contrib. Embryol. 38: 111-121; Schoenwolf et al. 1992. Dev. Dyn. 193: 235-248; Garcia-Martinez and Schoenwolf 1993. Dev. Biol. 159: 706-719). Precardiac cells residing in the primitive streak at stage 3 are uncommitted (Inagaki, et al., 1993. Dev. Dyn. 197: 57-68;) but become specified in response to signals from surrounding tissues after their migration into the lateral plate (Antin, et al. 1994. Dev. Dyn. 200: 144-154; Montgomery, et al., 1994. Dev. Biol. 164: 63-71; Sugi and Lough 1994. Dev. Dyn. 200: 155-162; Schultheiss, et al. 1995. Development 121: 4203-4214; Schultheiss, et al., 1997. Genes & Dev. 11: 451-462). The cardiac mesoderm precursors are in contact with presumptive anterior endoderm throughout their migration from the streak into the lateral plate (Garcia-Martinez and Schoenwolf 1993, supra). Anterior endoderm is required for cardiac specification in Xenopus (Nascone and Mercola 1995. Development 121: 515-523). Moreover, blood precursors from the posterior primitive streak develop into cardiac myocytes when cultured with anterior but not posterior endoderm (Schultheiss et al. 1995, supra). These findings suggest that the anterior endoderm secretes a heart-inducing signal that influences the fate of nascent mesodermal cells.

Bone morphogenetic protein (BMP) signals from the lateral regions of the embryo are also required for heart formation (Schultheiss et al. 1997, supra; Andreé, et al., 1998. Mech. Dev. 70: 119-131). The BMP antagonist noggin blocks cardiogenesis in explants of stage 4 precardiac mesoendoderm and blocks cardiogenesis in vivo when ectopically expressed through stage 7 (Schultheiss and Lassar 1997. Cold Spring Harbor Symp. Quant. Biol. 62: 413-419; Schultheiss et al. 1997, supra; Schlange, et al. 2000. Mech. Dev. 91: 259-270). Conversely, anterior paraxial mesoderm, which lies medial to the heart-forming region and normally gives rise to head mesenchyme, can be induced to express cardiac genes and to form beating cardiac myocytes in explant culture by exposure to BMP-2 at stages 5-6 (Schultheiss et al. 1997, supra; Andreé et al. 1998, supra). In vivo, implantation of a BMP-2-soaked bead into the anterior paraxial mesoderm induces the expression of Nkx-2.5 and GATA-4 (Schultheiss et al. 1997, supra; Schlange et al. 2000 Mech. Dev. 91: 259-270). While BMP signals can induce robust cardiac differentiation from anterior gastrula stage mesendoderm, posterior mesoderm fails to activate heart markers in response to BMP signals (Schultheiss et al. 1997, supra). These findings led us to propose a two-factor model for heart induction, in which a signal from the anterior endoderm induces a field of cardiogenic competence, and a BMP signal specifies the lateral portion of this field to develop into heart tissue (Schultheiss and Lassar 1997, supra; Schultheiss et al. 1997, supra).

Studies in Xenopus indicate that aspects of embryonic anteroposterior patterning are modulated by Wnt signals. Ectopic expression of FrzB, a Wnt-8 antagonist, expands cement gland and inhibits posterior development in Xenopus (Leyns, et. al. 1997. Cell 88: 747-756; Wang, et al., 1997. Cell 88: 757-766). In contrast, zygotically transcribed XWnt-8 promotes convergent extension movements and the development of ventral and posterior structures, including blood and somites (Christian and Moon 1993. Genes & Dev. 7: 13-28; Hoppler and Moon 1998. Mech. Dev. 71: 119-129; Hoppler, et. al. 1996. Genes &Dev. 10: 2805-2817). A second class of Wnt antagonists represented by Dkk-1 also inhibits Wnt-8 signaling at the extracellular level and has effects similar to those of FrzB on the Xenopus embryo (Glinka, et. al. 1998. Nature 391: 357-362;).

Although it is clear from these studies that modulation of Wnt signaling can control specification of anteroposterior identity in vertebrates, the effect of Wnt signaling on the induction of heart muscle has not yet been evaluated. Crescent is a member of the FrzB family of Wnt antagonists that is expressed in chick anterior endoderm during gastrulation, while this tissue displays heart-inducing activity (Schultheiss et al. 1995, supra; Pfeffer, et al., 1997. Int. J. Dev. Biol. 41: 449-458). During this period, cells in the primitive streak and posterior mesoderm express both Wnt-3a and Wnt-8c. The heart develops from mesoderm derived from the primitive streak, and thus, the cardiac precursor cells themselves expressed Wnt genes at an earlier stage of development.

The ability to produce heart cells, such as cardiomyocetes, has great importance for various therapeutic treatments, such as cell transplantation therapy as treatment for heart diseases or damage. We have now discovered that the inhibition of Wnt signaling plays a role in stimulating the differentiation of stem cells, e.g., such as cells derived from anterior mesoderm, embryonic stem cells, and side population cells, into cardiac cells. Accordingly, it is a method of the present invention to provide methods and compositions for enhancing the differentiation of stem cells into cardiac cells and/or enhancing the maintenance of cardiac cells.

### SUMMARY OF THE INVENTION

The invention provides methods and compositions for differentiating stem cells into differentiated cells, such as cardiac, kidney and liver cells.

In one aspect the invention provides a method for stimulating differentiation of stem cells into cardiac cells, comprising contacting a population of cells comprising stem cells with a sufficient amount of at least one Wnt antagonist and/or inhibitor to stimulate differentiation of the at least a portion of the stem cells into cardiac cells. In various embodiments, the Wnt antagonist may be an antagonist of Wnt signaling involving one or more of the following: Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16. In certain embodiments the Wnt antagonists may be a polypeptide, nucleic acid, or small molecule, including, but not limited to, a Dkk polypeptide, a crescent polypeptide, a cerberus polypeptide, an axin polypeptide, a Frzb polypeptide, a glycogen synthase kinase polypeptide, a T-cell factor polypeptide, or a dominant negative dishevelled polypeptide.

Stem cells used in associaiton with the methods and compositions described herein may be embryonic stem cells, adult stem cells, side population cells or germ cells. In certain embodiments, the cells may be isolated from a subject. For example, for treatment of a patient suffering from a heart disease, disorder or injury, the patient's own cells may be isolated and reintroduced into the patient after exposing the cells to a Wnt antagonist, or inhibitor, so as to stimulate the cells to differentiate into cardiac cells.

In certain embodiments of the methods and compositions described herein, it may be desirable to include a bone morphogenetic protein (BMP). Exemplary BMPs include, for example, BMP1, BMP2, BMP3, BMP4, BMP5, BMP6, BMP7, BMP8, BMP9, BMP10, BMP11, and BMP15.

In various embodiments of the invention, the Wnt antagonist polypeptides, BMP polypeptides, and stem cells may be of mammalian origin, e.g., human, mouse, rat, canine, feline, bovine, ovine, etc., or non-mammalian origin, e.g., from Xenopus, zebrafish, Drosophila, chicken, quail, etc.

In one embodiment, the invention provides a fragment of a Dkk protein that is at least about 5 times more potent than the full length Dkk protein in inducing differentiation of a stem cell into a cardiac cell. In another embodiment, the invention provides a polypeptide comprising a fragment of a Dkk protein comprising at most about 150, preferably 110 amino acids and a C-terminal cysteine rich domain. The polypeptide may be from a Dkk1 or Dkk2 protein. For example, the polypeptide may comprise about amino acids 159 to 266 of a Dkk1 protein, such as human Dkk1 having SEQ ID NO: 2. The polypeptide may further comprise a signal sequence, such as a signal peptide consisting of about amino acids 1 to 31 of SEQ ID NO: 2.

Isolated nucleic acids encoding such polypeptides are also within the scope of the invention. The nucleic acid may be linked to one or more transcriptional regulatory elements and may be part of a vector. The nucleic acid or vector may be in a host cell.

The invention provides methods for stimulating differentiation of stem cells into cardiac cells, comprising contacting a population of cells comprising stem cells with a Dkk protein or portion thereof sufficient to stimulate differentiation of a stem cell into a cardiac cell, such that the stem cells differentiate into cardiac cells. The Dkk protein may be Dkk1 or Dkk2, such as human Dkk1 or Dkk2 and may comprise SEQ ID NO: 2 or 4, or a portion thereof. The Dkk protein may be a fusion protein comprising an N-terminal cysteine rich domain of a Dkk1 protein and a C-terminal cysteine rich domain of a Dkk2 protein. In one embodiment, the method comprises contacting the population of cells with a fragment of a Dkk protein sufficient to stimulate differentiation of a stem cell into a cardiac cell. The fragment of the Dkk protein may comprise at most about 110 amino acids and a C-terminal cysteine rich domain, e.g., about amino acids 159 to 266 of SEQ ID NO: 2. The stem cells may be embyonic stem (ES) cells; side population (SP) stem cells or germ cells. The cells can be from a subject. In one embodiment, the method further comprises inhibiting LDL-receptor related protein (LRP) 6.

The invention also provides isolated cardiac cells, such as cells obtained according to a method of the invention. The invention also provides isolated population of differentiated cells, e.g., cardiac cells, wherein at least about 90% of the cells are differentiated cells, e.g., cardiac cells.

### BRIEF DESCRIPTION OF THE DRAWINGS

FIG. 1. Signals from the dorsal neural tube block cardiogenesis in anterior paraxial mesendoderm. (A) Whole-mount in situ hybridization for Nkx-2.5 in a stage 9 chick embryo (ventral side up). (B-D) show representative transverse sections as indicated in A. Anterior paraxial mesendoderm with overlying ectoderm (APMEE) is outlined in blue, APMEE with adjacent neural tube and notochord is outlined in green, and the anterior lateral mesendoderm with overlying ectoderm (ALMEE) is outlined in red. (E) A diagram of a stage 9 chick embryo is shown in the middle panel, and the APMEE is indicated by blue shading. Diagrams of transverse sections through APMEE explants cultured in either the presence or the absence of the axial tissues are shown on the left and right, respectively. (F) Diagram of APMEE explant cultured in the presence of only the dorsal neural tube. (G) RT-PCR analysis of gene expression in explants of APMEE that have been cultured in vitro for 48 h in either the presence (lane 1) or the absence (lane 2) of the adjacent axial tissues or cultured in the presence (lane 3) or the absence (lane 4) of only the dorsal neural tube. Cardiogenesis was observed in 33 of 48 APMEE explants cultured in the absence of the axial tissues and was never observed in APMEE explants cultured in the presence of the axial tissues. APMEE explants were cultured in either the presence (lane 5) or absence (lane 6) of the BMP antagonist, noggin. Noggin administration blocked cardiogenesis in 10 out of 14 APMEE explants. Alternatively, explants of APMEE plus the neural tube and notochord (lane 7), APMEE alone (lane 8), or anterior lateral mesendoderm plus ectoderm (ALMEE; lane 9) were dissected and immediately harvested for RNA. Transcript levels of the indicated genes were monitored by RT-PCR analysis. Similar results were obtained in four independent experiments.

FIG. 2. Inhibition of cardiogenesis by Wnt-1 and Wnt-3a. Expression of Wnt-l (A) and Wnt-3a (B) as assessed by whole-mount in situ hybridization in stage 9-10 chick embryos. (C) Stage 9 APMEE explants were infected with either RCAS-Wnt-3a (lane 1) or RCAS-AP (lane 2). Rat-1/Wnt-1-HA cells (lane 3) or Rat-I control cells (lane 4) were cocultured with APMEE explants on raft filters. Wnt-3a expression blocked cardiogenesis in ˜70% of the APMEE explants (n=24), whereas Wnt-1 expression blocked cardiogenesis in 50% of such cultures (n=12). (D-G) Rat-1/Wnt-1 cells were transiently transfected with either a control IgG expression vehicle (D, E) or a Frzb-IgG expression vehicle (F, G). Cell pellets were implanted into the left side of the heart-forming region of a stage 7 chick embryo that was maintained in New culture. Embryos were allowed to developed to stage 9-10, and subsequently analyzed by whole-mount in situ hybridization for Nkx-2.5 gene expression (D, F). Following in situ hybridization, the embryos were subsequently immunostained for IgG to identify the location of the IgG- or the Frzb-IgG-expressing cell pellet (E, G). The IgG- or the Frzb-IgG-expressing cells stain darker purple than cells expressing Nkx-2.5 as detected by in situ hybridization in D and F. (H) APMEE explants were cultured either with Rat-1/Wnt-3a-HA cells (lanes 1, 2) or Rat-1/Wnt-1-HA cells (lanes 3, 4) that had been transiently transfected with either control IgG (lanes 1, 3) or with Frzb-IgG (lanes 2, 4). After 48 h in culture, RNA was harvested and transcript levels of the indicated genes were monitored by RT-PCR analysis. Similar results were obtained in three independent experiments. Western blot analysis of the expression levels of the HA-tagged Wnts is shown.

FIG. 3. A combination of BMP signals and Frzb promotes cardiogenesis in anterior paraxial mesendoderm in the presence of the neural tube and notochord. (A) Stage 8 APMEE plus the adjacent neural tube and notochord (schematically illustrated in FIG. 1E) were dissected. Explants were cultured in the presence of either soluble control IgG (lanes 1, 3) or soluble Frzb-IgG (lanes 2, 4) in either the absence (lanes 1, 2) or presence (lanes 3, 4) of 60 ng/mL BMP-2. Alternatively, APMEE plus neural tube and notochord explants were cocultured on raft filters with aggregates of 293 cells transfected with either a control IgG expression vehicle (lanes 5, 7) or a Frzb-IgG expression vehicle (lanes 6, 8) and cultured either in the absence (lanes 5, 6) or the presence (lanes 7, 8) of 60 ng/mL BMP-2. After 48 h in culture, RNA was harvested and transcript levels monitored by RT-PCR. (B) Analysis of gene expression in APMEE explants that have been cultured in the presence of either the dorsal neural tube plus the floor plate and notochord (lane 1) or the dorsal neural tube only (lanes 2-5; schematically illustrated in FIG. 1F). Explants were cultured in the presence of exogenous BMP-2 or Frzb-IgG (lanes 3 and 5, respectively). Similar results have been obtained in four independent experiments.

FIG. 4. Inhibiting Wnt signals in vivo directs anterior paraxial mesodermal cells into the cardiac fate. (A) Pellets of cells expressing BMP-4, Frzb-IgG, and/or control IgG were implanted into the left side of the anterior paraxial mesoderm in stage 7 chick embryos. The location of the cell pellet is represented by a blue dot. In some cases, DiI was subsequently injected into a region that lay medial to the implanted cell pellet (red stars). The embryo is depicted dorsal side up. (B, C) Whole-mount in situ hybridization for vMHC expression is shown in stage 12 embryos that had been previously implanted with cell pellets expressing BMP-4 plus control IgG (B) or the combination of Frzb-IgG and BMP-4 (C). (D-F) Examples of the results obtained from DiI-injected embryos; dorsal side up. HEK-293 cells transfected with the indicated plasmids were implanted as described in A. Embryos were allowed to developed to stage 12-13 (26-30 h) before fixation. Brightfield and fluorescent images were taken and are overlayed. (G) Statistical summary of the in vivo results (red, DiI tracing; black, heart looping). (H,) Implanting Frzb-IgG- and BMP-4-expressing cells into the anterior paraxial mesoderm induces migration of cells within this tissue into regions of the heart that express vMHC. Transverse section of embryo implanted with Frzb-IgG- and BMP-4-expressing cells in the anterior paraxial mesoderm (as shown in F). DiI fluorescence signals were photoconverted into a brown precipitate before in situ hybridization for vMHC. (1) High-power magnification of the square area outlined in H. DiI-labeled cells are brown (indicated by arrow heads in 1); vMHC-positive cells stain blue; (nt) neural tube; (nc) notochord; (da) dorsal aorta; (ve) heart ventricle.

FIG. 5. Heart formation is cued by a combination of positive and negative signals from surrounding tissues. Whereas a signal(s) from the anterior endoderm works to promote heart formation in concert with BMP signals in the anterior lateral mesoderm (blue arrows), signals from the axial tissues (red) repress heart formation in the more dorsomedial anterior paraxial mesoderm. Inhibitory signals that block heart formation in anterior paraxial mesoderm include Wnt family members expressed in dorsal neural tube (Wnt-1 and Wnt-3a) and anti-BMPs expressed in the axial tissues (i.e., noggin in the notochord).

FIG. 6. dkk-1 and crescent, but not frzb, induce cardiac specific gene expression in noncardiogenic tissue. (A) mRNAs encoding various Wnt and BMP antagonists were injected equatorially into ventral blastomeres at the four-cell stage. Ventral marginal zone (VMZ) tissue was then explanted from Xenopus laevis at stage 10 and cultured until analyzed by RT-PCR for gene expression at stage 30 (see Materials and Methods). (B) Injection of dkk-1 or crescent induced both markers of cardiac mesoderm (Tbx5 and Nkx2.5) and heart muscle-specific genes (cardiac isoform of troponin-I, Tnlc, and myosin heavy chain-α, MHCα) in VMZ tissue. frzb, in contrast, induced muscle actin (m. actin), which is primarily a skeletal muscle marker, but not cardiac specific gene expression. Induced genes were expressed at levels comparable to endogenous expression in control dorsal marginal zone (DMZ) explants. (C-E) TnIc transcripts induced by injection of 1.5 ng of dkk-1 or crescent mRNAs were highly localized, similar to endogenous expression (cf. with control DMZ shown in FIGS. 8C and 10G), whereas injection of frzb mRNA does not induce TnIc. (F, G) dkk-1, crescent, and frzb block Wnt8 induction of Siamois in animal cap tissue. Wnt8 and Wnt antagonist mRNAs were injected into the animal region of two-cell-stage embryos and caps were isolated at stage 9, cultured, and processed for RT-PCR at stage 10.5 (F). Antagonism of Wnt8 signaling indicates that functional protein is translated from the injected mRNAs in each case (G). EF1α expression is shown as a control for the RT reaction in all cases.

FIG. 7. Injection of the Wnt antagonists dkk-1 and crescent resulted in the formation of beating hearts in VMZ tissue. Embryos were injected ventrally with 900 pg dkk-1, 1.5 ng crescent, or 1.5 ng frzb mRNA at the four-cell stage, and VMZ explants isolated and cultured as above. (A) The explants were scored for rhythmic beating when sibling controls reached stage 41. Uninjected VMZ and DMZ explants were analyzed as negative and positive controls, respectively. (B-D) Control DMZ explants formed an embryoid-like structure having a well-developed anteroposterior body axis (B). The heart tube contained a myocardial layer that stained with CT-3, which recognizes the cardiac isoform of troponin-T (C), lined by a thin layer of CT-3 negative endothelial cells visualized with DAPI (arrow in D). (E-G) dkk-1 injected VMZ explants formed simple structures resembling a small epithelial sac encapsulating a CT-3 positive myocardial tube (F) also lined by endothelial cells (G). (H-J) crescent-injected VMZs formed similar structures. Pigmented melanocytes were seen scattered on the surface of the dkk-1- and crescent-injected VMZ explants, and cement gland tissue was often observed (cluster of pigmented cells on surface of tissue in E). Line represents 25 μm.

FIG. 8. Induction of cardiogenesis in the VMZ assay is specific to certain Wnt antagonists. (A) The BMP antagonists Noggin and Chordin did not induce specific markers of cardiogenesis (TnIc or MHCα) despite induction of m. actin and elongation of the explants (not shown). Noggin did not induce Tbx5, Nkx2.5, or Nkx2.10, whereas chordin weakly induced these genes. Note that Tbx5 and Nkx2.5 are expressed in tissues other than cardiac mesoderm and that induction of these genes (in the absence of other markers) does not necessarily indicate heart field specification (see text). (B) Wnt antagonists not normally present in gastrula-stage embryos induced weak expression of Tbx5, Nkx2.5, and Nkx2.10 but did not induce the more specific cardiac markers TnIc or MHCα. In situ hybridization for expression of Nkx2.5 (C-J) and TnIc (C′-J′) indicated that only WIF-1 induced detectable levels of Nkx2.5 expression (arrow in H; 4 of 24 explants showed expression) and that none of these mRNAs induced TnIc. Arrowheads in F and F′ show pigmented cement glands that formed in explants injected with chordin mRNA.

FIG. 9. Injection of mRNA encoding GSK3β is sufficient to induce both markers of cardiac mesoderm and heart muscle-specific proteins, indicating that inhibition of β-catenin signal transduction is sufficient to induce cardiogenesis in the VMZ assay.

FIG. 10. Overexpression of Wnt3A and Wnt8, but not Wnt5A and Wnt11, blocks endogenous expression of Nkx2.5 and TnIc in DMZ tissue. (A) Expression was targeted to the heart-forming region by injection of a plasmid encoding Wnt cDNA into dorsal blastomeres at the four-cell stage. DMZ explants were dissected at stage 10 and analyzed when sibling controls reached stage 23 (Nkx2.5) or stage 30 (TnIc). (B) Percentage of explants expressing Nkx2.5 and TnIc as determined by in situ hybridization. (C-G) Examples of TnIc in situ hybridization patterns in DMZ explants overexpressing Wnt cDNAs. Note that nearly all control DMZ explants expressed both markers (G), as did DMZ explants overexpressing Wnt5A and Wnt11 (E, F). In contrast, Wnt3A and Wnt8 reduced the incidence of Nkx2.5 and TnIc expression (B). Whereas Nkx2.5 expression was lost entirely in affected explants, TnIc expression was either absent (C, D) or greatly reduced in area (C′, D′).

FIG. 11. Crescent is expressed anteriorly, whereas Wnt-8c and Wnt-3a are expressed posteriorly in gastrula stage chick embryos. In situ hybridization comparing crescent (A-C), Wnt-8c (D-F), and Wnt-3a (G-1) expression patterns at the indicated gastrulation stages. (C, F, I) Sections of stage 6 embryos are at the levels indicated by the red lines in B, E, and H, respectively. Crescent expression is restricted to the germinal crescent, anterior endoderm, and prechordal plate. Wnt-8c is expressed in primitive streak and migrating lateral plate mesoderm. Wnt-3a is expressed in the epiblast of the primitive streak. Arrow in F shows expression of Wnt-8c in lateral plate mesoderm.

FIG. 12. Crescent is an efficient Wnt-8 antagonist. (A) Crescent injection into one cell of a two-cell embryo enlarged anterior structures and inhibited posterior extension in injected Xenopus embryos (bottom series of embryos). Control embryos (top) were injected with globin mRNA. LacZ mRNA was included as a lineage tracer. (B) Crescent inhibited the induction of siamois by chick Wnt-8c in Xenopus animal caps. RT-PCR analysis of siamois and ornithine decarboxylase (ODC) expression in whole embryos (WE; lane 1) or animal caps from embryos injected with the following RNAs: globin RNA (lane 2), 1 ng of crescent RNA (lane 3), 200 pg of chick Wnt-8c RNA (lane 4), 200 pg of chick Wnt-8c and 1 ng of crescent RNA (lane 5), 10 pg of mWnt-3a (lane 6), or 10 pg of mWnt-3a and 1 ng of crescent (lane 7). All injected RNA was made up to 1.2 ng with globin RNA. The difference between the levels of siamois expression in lanes 6 and 7 was approximately threefold, when normalized to ODC levels.

FIG. 13. Stage 5 chick posterior lateral plate and posterior primitive streak express heart markers when cocultured with quail anterior endoderm. Stage 5 chick PLP mesoderm was explanted and cultured either alone (lane 1) or in the presence of quail anterior endoderm (lane 2). Stage 5 chick PPS was explanted and cultured either alone (lane 3) or in the presence of quail anterior endoderm (lane 4). Cultures were grown for 48 h and harvested for RNA. Gene expression for GAPDH, Nkx-2.5, vMHC, and aMHC were assayed for both quail (Q) and chick (C) tissue by RT-PCR. Restriction site polymorphisms were employed to distinguish quail and chick transcripts.

FIG. 14. Wnt antagonists can induce cardiogenesis in PLP mesoderm but not in PPS explants. (A) Stage 5 posterior lateral plate (PLP) mesoderm (lanes 1, 2) or posterior primitive streak (lanes 3, 4) were infected with RCAS viruses encoding either alkaline phosphatase (AP;

lanes 1, 3) or crescent (lanes 2, 4). Gene expression for the indicated genes was assayed by RT-PCR analysis. (B) Time course of Wnt-8c and Wnt-3a expression in stage 5 PLP and PPS mesoderm explants. PLP mesoderm (lanes 1-4) or PPS (lanes 5-8) were cultured for the indicated periods of time. At the end of the culture period, explants were harvested and transcript levels evaluated by RT-PCR. (C) Posterior tissues cocultured with COS cells expressing pCS2+-β-gal, pCMV-Dkk-1 (Xenopus), or pCS2+-crescent. PLP mesoderm (lanes 1, 2, 5, 6) or PPS (lanes 3, 4, 7, 8) were cultured with either control COS cells expressing CS2⁺-β-gal (lanes 1, 3, 5, 7), COS cells expressing pCMV2-XDkk-1 (lanes 2, 4), or COS cells expressing pCS2+-crescent (lanes 6, 8). Transcript levels for the indicated genes were evaluated by RT-PCR.

FIG. 15. Overexpression of Wnt genes blocks cardiogenesis in precardiac mesoderm. (A) Whole-mount in situ hybridization for Nkx-2.5 in chick embryos in which pellets of chick embryo dermal fibroblasts infected with either RCAS-mWnt-3a or control RCAS-AP were implanted into the precardiac region of embryos in New culture at stage 3⁺ to 4. Pellets of RCAS-Wnt-3a infected CEFs (red arrowheads) inhibit expression of Nkx-2.5 in a stage 9 embryo whereas pellets of control RCAS-AP-infected CEFs (open arrowheads) do not. (B) Red line in A indicates the level of this section. RCAS-Wnt3a-expressing cell pellets (red dotted circle) but not control cell pellets (black dotted circle) inhibit expression of cNkx-2.5 in the precardiac mesoderm and foregut endoderm but do not inhibit the accumulation of mesoderm lateral and ventral to the neural tube. (C) Ectopic Wnt expression suppresses cardiogenesis in anterior lateral plate mesoderm explants. Stage 5 anterior lateral plate mesoderm from the precardiac region was infected with either control virus (RCAS-GFP, lane 1; RCAS-AP, lane 3), or RCAS-Wnt-3a (lane 2) or RCAS-Wnt-8c (lane 4). Cultures were carried out in the presence of 200 ng/mL BMP-4 overnight followed by 48 h in 20 ng/mL BMP-4. Transcript levels were evaluated by RT-PCR.

FIG. 16. Shows the nucleotide sequence of human Dkk1 (SEQ ID NO: 1).

FIG. 17. Shows the nucleotide sequence of human Dkk 2 (SEQ ID NO: 3).

FIG. 18. Show the amino acid sequence of human Dkk1 (SEQ ID NO: 2) and the location of the N-terminal and C-terminal cysteine rich sequences and signal sequence. The cysteine rich domains are indicated by continuous lines. Alternative cysteine rich domains are indicated by stippled lines.

FIG. 19. Shows the amino acid sequence of human Dkk2 (SEQ ID NO: 4) and the location of the N-terminal and C-terminal cysteine rich sequences and signal sequence.

FIG. 20. Shows the amino acid sequence for Dkk fragments C1 (SEQ ID NO: 5) and C2 (SEQ ID NO: 6) as described in the Examples. For both the C1 and the C2 fragments, the underlined portion of the sequence represents the singal peptide from Dkk1 and the double underlined portion of the sequence represents the Flag epitope tag. The non-underlined portion of C1 represents amino acids 156-266 of Dkk1 as shown in FIG. 18 and the non-underlined portion of C2 represents amino acids 151-259 of Dkk2 as shown in FIG. 19.

FIGS. 21A and B. Shows enhancement of cardiomyogenesis by recombinant Dkk1.

FIG. 22. Shows FACS profiles of Hoechst 33342 and propidium iodide treated quail cells, showing the presence of SP cells within the boxed region.

### DETAILED DESCRIPTION OF THE INVENTION

Wnts are encoded by a large gene family whose members have been found in round worms, insects, cartilaginous fish and vertebrates. Wnts are thought to function in a variety of developmental and physiological processes since many diverse species have multiple conserved Wnt genes (McMahon, Trends Genet., 8: 236-242 [1992]; Nusse and Varmus, Cell, 69: 1073-1087 [1992]). Wnt genes encode secreted glycoproteins that are thought to function as paracrine or autocrine signals active in several primitive cell types (McMahon, supra [1992]; Nusse and Varmus, supra [1992]). The Wnt growth factor family includes more than 10 genes identified in mouse and human (Wnt-1, 2, 2b, 3, 3a, 4, 5a, 5b, 6, 7a 7b, 8a, 8b, 10a, 10b, 11, 14, 16) (see, e.g., Gavin et al., Genes Dev., 4: 2319-2332 [1990]; Lee et al., Proc. Natl. Acad. Sci. USA, 92: 2268-2272; Christiansen et al., Mech. Dev. 51: 341-350 [1995], Vant Veer et al., Mol. Cell. Biol., 4: 2532-2534 [1984]).

Studies of mutations in Wnt genes have indicated a role for Wnts in growth control and tissue patterning. In Drosophila, wingless (wg) encodes a Wnt gene (Rijsenijk et al., Cell. 50: 649-657 [1987]) and wg mutations alter the pattern of embryonic ectoderm, neurogenesis, and imaginal disc outgrowth (Morata and Lawrence, Dev. Biol., 56: 227-240 [1977]; Baker, Dev. Biol., 125: 96-108 [1988]; Klingensmith and Nusse, Dev. Biol., 166: 396-414[1994]). In Caenorhabditis elegans, lin-44 encodes a Wnt which is required for asymmetric cell divisions (Herman and Horvitz, Development, 120: 1035-1047 [1994]). Knock-out mutations in mice have shown Wnts to be essential for brain development (McMahon and Bradley, Cell, 62: 1073-1085 [1990]; Thomas and Cappechi, Nature, 346: 847-850 [1990]), and the outgrowth of embryonic primordia for kidney (Stark et al., Nature, 372: 679-683 [1994]), tail bud (Takada et al., Genes Dev., 8: 174-189 [1994]), and limb bud (Parr and McMahon, Nature, 374: 350-353 [1995]). Overexpression of Wnts in the mammary gland can result in mammary hyperplasia (McMahon, supra (1992]; Nusse and Varmus, supra [1992]), and precocious alveolar development (Bradbury et al., Dev. Biol., 170: 553-563 [1995]).

There are a variety of proteins which have also been shown to be Wnt antagonists, including, for example the Dkk, crescent, cerberus, axin, Frzb, GSK, TCF, dominant negative dishevelled, dominant negative N-cadherin, and dominant negative β-catenin polypeptides.

Frizzled proteins are seven-transmembrane proteins that act as receptors for Wnt proteins. The extracellular part of the receptor which binds to the Wnt is referred to as the cystein-rich domain or CRD. Polypeptides comprising the CRD of frizzleds are secreted proteins referred to as FRP/FrzB molecules and can act as antagonists of Wnt signaling. There are various Frizzled proteins which have been identified in the mouse (e.g., Fzd1, Fzd2-rs1, Fzd2-rs2, Fzd3, Fzd4, Fzd5, Fzd6, Fzd7, Fzd8, Fzd9, and Smoh), human (e.g., FZD1, FZD2, FZD3, FZD4, FZD5, FZD6, FZD7, FZD8, FZD9, FZD10, and SMOH) and rat (e.g., Rfz1 and Rfz2). Examples of FRP/FrzB polypeptides include FRP-1, SARP2, FrzA, FRP-2, SDF-5, SARP-1, FRP-3, FrzB, Fritz, FRP-4, frpAP, frpHE, SARP3, and sizzled.

Dickkopf (Dkk) proteins are cystein rich secreted proteins that have been shown to be negative regulators of Wnt signaling. Dkk does not bind directly to Wnt but acts via the Wnt co-receptor LRP (LDL-receptor related proteins LRP5 and LRP6). Four Dkk proteins have been identified in humans referred to as Dkk1-4. Dkks are composed of two cysteine-rich domains separated by a variable length spacer region. Both domains are well conserved among all four members of the Dickkopf family (Glinka, et al., 1998. Nature 391:357-362; Krupnik, et al. 1999. Gene 238:301-313). In particular, Dkk1 and Dkk2 share 50% identity in their N-terminal cysteine-rich region, and 70% identity in their C-terminal regions. Dkk family members are expressed throughout development in a tissue- and stage-restricted manner. Their transcripts are found in the brain, heart, lungs, limbs, and other tissues in which epithelial-mesenchymal interactions occur (Grotewold, et al., 1999. Mech. Dev. 89:151-153; Krupnik, et al. 1999, supra; Monaghan, et al., 1999. Mech. Dev. 87:45-56), suggesting that these proteins modulate a number of important developmental processes. Dkk1, the most extensively studied Dickkopf family member, is a potent Wnt antagonist (Glinka, et al., 1998, supra; McMahon and Moon 1989. Cell 58:1075-1084; Smith and Harland 1991. Cell 67:753-765; Sokol 1991. Cell 67:741-752). Various functional and structural studies involving the Dkk proteins have been carried out and C-terminal fragments of Dkks are sufficient to inhibit Wnt8 (Krupnik et al., Gene 238: 301-13 (1999); Brott & Sokol, Mol. Cell Biol. 22: 6100-10 (2002)).

Dishevelled proteins (Dsh) interact with a variety of proteins essential in Wnt signaling including the Casein Kinase 1 and 2 proteins. Various Dsh proteins have been identified in human (DVL1, DVL2, DVL3, and Dvl1L1), mouse (Dvl-1, Dvl-2, and Dvl-3), drosophila (Dsh), and C. elegans (mig-5).

Axin associates with β-catenin, GSK-3b and APC via various domains in the axin protein. Overexpression of axin in Xeonpus embryos destabilizes β-catenin and blocks the axis-duplicating activity of XWnt-8. Actin proteins have been cloned in mouse, human (AXIN1 and AXIN2), Xenopus, zebrafish, Drosophila and C. elegans.

GSK-3 plays a role in Wnt signalling by inducing β-catenin degredation via phosphorylation of the molecule. GSK3 has been cloned in human (GSK3 alpha and beta), mouse and Drosophila.

T-cell factor/lymphoid enhancer factor (TCF/LEF) proteins mediate Wnt signaling in the nucleus via transcriptional activation/repression of Wnt targets. Various TCF proteins have been identified in human (TCF1, 3 and 4), mouse, chicken and Xenopus.

In Xeonpus embryos, cerberus is expressed in the head organizing region that consists of crawling-migrating cells. The cerberus expressing region corresponds to the prospective foregut, including the liver and pancreas anlage, and the heart mesoderm. Cerberus expression is activated by chordin, noggin, and organizer-specific homeobox genes.

The invention is based at least in part on the discovery that Wnt antagonists, including, but not limited to, the Dkk proteins, stimulate the differentiation of stem cells into cardiac cells. The invention is also based on the discovery that fragments of Dkk proteins that comprises the C-terminal cysteine rich domain are even more potent in inducing differentiation of stem cells into cardiac cells relative to a full length Dkk protein.

Definitions

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

The term “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

A “cysteine rich domain” refers to a domain in a Wnt antagonist protein that is rich in cysteine residues. In an exemplary embodiment, cysteine rich domain refers to a domain in a Dkk protein that is rich in cysteine residues. Dkk proteins usually have two cysteine rich domains. The domain located closer to the N-terminus of the protein is referred to as the “N-terminal cysteine rich domain,” whereas the domain that is located closer to the C-terminus of the protein is referred to as the “C-terminal cysteine rich domain.” The N-terminal cysteine rich domain of human Dkk1 consists of about amino acids 97-138 or about amino acids 85-138 of SEQ ID NO: 2 (Fedi et al. (1999) J. Biol. Chem. 274:19465 and Krupnik et al. (1999) Gene 238:301; FIG. 18). The C-terminal cysteine rich domain of human Dkk1 consists of about amino acids 183-245 or about amino acids 189-266 of SEQ ID NO: 2 (Fedi et al., supra and Krupnik et al., supra; FIG. 18). The N-terminal cysteine rich domain of human Dkk2 consists of about amino acids 78-127 of SEQ ID NO: 4 (Krupnik et al., supra; FIG. 19). The C-terminal cysteine rich domain of human Dkk2 consists of about amino acids 183-259 of SEQ ID NO: 4 (Krupnik et al., supra; FIG. 19). The location of each of the domains in other Dkk proteins in other species and/or in other Dkk proteins can be derived by amino acid sequence comparisons. Domains in other species and Dkk proteins are also described in Krupnik et al., supra.

A “delivery complex” refers to a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

The term “derivative” of a polypeptide or polynucleotide refers to a chemically modified polypeptide or polynucleotide. Chemical modifications of a polynucleotide can include, for example, replacement of hydrogen by an alkyl, acyl, or amino group. A derivative polynucleotide encodes a polypeptide which preferably retains at least one biological or immunological function of the natural molecule. A derivative polypeptide is one modified by glycosylation, pegylation, phosphorylation or any similar process that retains at least one biological or immunological function of the polypeptide from which it was derived.

The term “differentiating Wnt antagonist” refers to a Wnt antagonist which is known, or has been shown (e.g., such as by the assays described herein), to induce differentiation of a stem cell into a cardiac cell. Examples of a differentiating Wnt antagonist, include, for example, Dkk and crescent. An example of a Wnt antagonist which is not a differentiating Wnt antagonist is a dominant negative Wnt8 protein (see e.g., Hoppler et al., Genes & Dev. 10: 2805-2817 (1996)).

“Dkk protein” refers to a protein of the Dkk family of proteins that contains one or more cysteine-rich domains. The Dkk family of proteins includes Dkk1, Dkk2, Dkk3 and Dkk4, and any other protein sufficiently related to one or more of these proteins at the sequence level, structurally or functionally. This family of proteins is described, e.g., in Krupnik et al. (1999) Gene 238:301. Human Dkk1 is a protein of 266 amino acids; human Dkk2 is a protein of 259 amino acids; human Dkk3 is a protein of 224 amino acids and human Dkk4 is a protein of 350 amino acids. Human Dkk1 and Dkk2 nucleotide sequences are set forth as SEQ ID NO: 1 and 3, respectively. Human Dkk1 and Dkk2 amino acid sequences are set forth in FIGS. 18 and 19 and as SEQ ID NO: 2 and 4, respectively. Nucleotide and amino acid sequences of Dkk nucleic acids and proteins from various species can be found, e.g., under the following GenBank numbers:

| | nucleic acid | protein |
|---|---|---|
| HumanDkk1 | NM_012242; NT_024082; | NP_036374; O94907; |
| | AH009834; NT_024082 | AAG15544 |
| Mouse Dkk1 | NM_010051 | O54908; NP_034181 |
| Zebrafish Dkk1 | AF116852; AB023488 | ADD22461; BAA82135 |
| Human Dkk2 | NM_014421; NT_006397; | NP_064661; NP_055236; |
| | XM_003612 | XP_003612; Q9UBU2 |
| Mouse Dkk2 | NM_120265 | Q9QYZ8; NP_064661 |
| Xenopus Dkk2 | AJ300197 | CAC17815 |
| Human Dkk3 | NM_015814; | NP_056965; NP_037385; |
| | NM_0132253 | Q9UBP4 |
| Mouse Dkk3 | NM_015814; AK013622; | NP_056629; Q9QUN9 |
| | AK004853; AK013054 | |
| Chick Dkk3 | | Q90839 |
| Human Dkk4 | NT_017505; NM_014420 | NP_055235; Q9UBT3 |

Allelic variants and mutants of Dkk proteins such as those recited herein are also encompassed by this definition.

“Dkk reagents” include Dkk proteins, fragments thereof, homologs thereof, derivatives thereof and peptidomimetics thereof that are capable of stimulating the differentiation of stem cells into differentiated cells, e.g., cardiac cells, kidney cells or liver cells.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

A “homolog” of a Dkk protein or fragment thereof refers to a polypeptide having a significant amino acid sequence homology to the Dkk protein or fragment thereof. For example, a homolog can have an amino acid sequence that is at least about 70%; preferably at least about 80%; 90%; 95%; 98%; or 99% identical or similar to that of the Dkk protein or fragment thereof. Homologs may have at most 20; 15; 10; 5; 3; 2 or 1 amino acid deletions, additions or substitutions. Substitutions may be conservative or non-conservative substitutions. A homolog of a Dkk protein or fragment thereof can also be a polypeptide that is encoded by a nucleic acid that hybridizes under stringent conditions to a nucleic acid that encodes the Dkk protein or fragment thereof.

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes which contain certain mismatches, provided that the two strands are still forming a double stranded helix. “Stringent hybridization conditions” refers to hybridization conditions resulting in essentially specific hybridization. The term “specific hybridization” of a probe to a target site of a template nucleic acid refers to hybridization of the probe predominantly to the target, such that the hybridization signal can be clearly interpreted. As further described herein, such conditions resulting in specific hybridization vary depending on the length of the region of homology, the GC content of the region, the melting temperature “Tm” of the hybrid. Hybridization conditions will thus vary in the salt content, acidity, and temperature of the hybridization solution and the washes.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. An “isolated cell” or “isolated population of cells” is a cell or population of cells that is not present in its natural environment.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wis., Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases. Databases with individual sequences are described in Methods in Enzymology, ed. Doolittle, supra. Databases include Genbank, EMBL, and DNA Database of Japan (DDBJ).

The term “protein” is used interchangeably herein with the terms “peptide” and “polypeptide.”

A “stem cell” refers to a cell that is capable of differentiating into a desired cell type. A stem cell includes embryonic stem (ES) cells; adult stem cells; and somatic stem cells, such as SP cells from uncommitted mesoderm. A “totipotent” stem cell is capable of differentiating into all tissue types, including cells of the meso-, endo-, and ecto-derm. A “multipotent” or “pluripotent” stem cell is a cell which is capable of differentiating into at least two of several fates.

The term “stimulating” with reference to differentiation of a stem cell into a cardiac cell, is meant to encompass any change in a stem cell which increases the likelihood that the cell will progress toward becoming a cardiac cell as compared to what would occur in the absence of such changes. Such differentiation may be monitored by a variety of means, including, for example, visually (e.g., by inspecting the cell, cell population, or tissue under a microscope), electically (e.g., by measuring changes in electrical potential of the cell or cell surface), mechanically (e.g., by measuring changes in cell length or volume), or biochemically (e.g., by assaying for the presence of one or more gene and/or protein markers). In certain embodiments, stimulation of differentiation will have the effect of priming the cell or causing a partial differentiation of the cell toward a cardiac cell which differentiation may be completed upon exposure to another factor. In other embodiments, stimulation of differentiation will lead to full differentiation of at least a portion of the stem cells in a cell population into cardiac cells.

The term “test compound” refers to a molecule to be tested by one or more screening method(s) for its ability to stimulate differentiation of stem cells into cardiac cells. Examples of test compounds include, but are not limited to, peptides, nucleic acids, carbohydrates, and small molecules.

A “variant” of a polypeptide refers to a polypeptide having the amino acid sequence of the peptide which is altered in one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. The polypeptides generally will have significant amino acid identity relative to each other. A polymorphic variant is a variation in the polynucleotide sequence of a particular gene between individuals of a given species. Polymorphic variants may encompass “single nucleotide polymorphisms” (SNPs) in which the polynucleotide sequence varies by one base. The presence of SNPs may be indicative of, for example, a certain population, a disease state, or a propensity for a disease state.

The term “Wnt antagonist” refers to a molecule or composition which downregulates (e.g., suppresses or inhibits) signal transduction via the Wnt pathway. Downregulation may occur directly, e.g., by inhibiting a bioactivity of a protein in a Wnt signaling pathway, or indirectly, e.g., by inhibiting downsteam mediators of Wnt signaling (such as TCF3) or by decreasing stability of β-catenin, etc. Examples of Wnt antagonists include, but are not limited to, Dkk polypeptides (Glinka et al., Nature (1998) 391: 357-62; Niehrs, Trends Genet (1999) 15(8):314-9), crescent polypeptides (Marvin et al., Genes & Dev. 15: 316-327 (2001)), cerberus polypeptides (U.S. Pat. No. 6,133,232), axin polypeptides (Zeng et al., Cell (1997) 90(1):181-92; Itoh et al., Curr Biol (1998) 8(10):591-4; Willert et al., Development (1999) 126(18):4165-73), Frzb polypeptides (Cadigan et al., Cell (1998) 93(5):767-77; U.S. Pat. No. 6,133,232; U.S. Pat. No. 6,485,972), glycogen synthase kinase (GSK) polypeptides (He et al., Nature (1995) 374(6523): 617-22), T-cell factor (TCF) polypeptides (Molenaar et al., Cell (1996) 86(3):391-9), dominant negative dishevelled polypeptides (Wallingford et al., Nature (2000) 405(6782): 81-5), dominant negative N-cadherin polypeptides (U.S. Pat. No. 6,485,972), dominant negative β-catenin polypeptides (U.S. Pat. No. 6,485,972), dominant negatives of downstream transcription factors (e.g., TCF, etc.), dominant negatives of Wnt polypeptides, agents that disrupt LRP-frizzled-wnt complexes, and agents that sequester Wnts (e.g., crescent and antibodies to Wnts). Wnt antagonist polypeptides may be of mammalian origin, e.g., human, mouse, rat, canine, feline, bovine, or ovine, or non-mammalian origin, e.g., from Xenopus, zebrafish, Drosophila, chicken, or quail. Wnt antagonists also encompass fragments, homologs, derivatives, allelic variants, and peptidomimetics of various polypeptides, including, but not limited to, Dkk, crescent, cerberus, axin, Frzb, GSK, TCF, dominant negative dishevelled, dominant negative N-cadherin, and dominant negative β-catenin polypeptides. In other embodiments, Wnt antagonists also include antibodies (e.g., Wnt-specific antibodies), polynucleotides and small molecules.

Polypeptides and Polypeptidomimetics of the Invention

The invention provides polypeptides that are capable of stimulating the differentiation of stem cells into cardiac cells. In exemplary embodiments, the polypeptides of the invention are Wnt antagonists such as Dkk, crescent, cerberus, axin, Frzb, GSK, TCF, dominant negative dishevelled, dominant negative N-cadherin, and dominant negative β-catenin polypeptides, and fragments, homologs, derivatives, allelic variants, and peptidomimetics thereof. In an exemplary embodiment, the polypeptide is a Dkk protein or a fragment thereof. The Dkk protein can be of mammalian origin, e.g., human, mouse, rat, canine, feline, bovine, ovine. The protein can also be of non-mammalian origin, e.g., from Xenopus, Zebrafish, drosophila, chicken, or quail. In a preferred embodiment, the Dkk protein is Dkk1 or Dkk2. In an even more preferred -embodiment, the protein is human Dkk1 or Dkk2, e.g., proteins comprising, or consisting of, the amino acid sequence set forth in SEQ ID NO: 2 or 4.

In another embodiment, the polypeptides of the invention comprise a fragment of a Wnt antagonist. A fragment of a, Wnt antagonist refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. In exemplary embodiments, fragments of Wnt antagonists retain the ability to induce differentiation of stem cells into cardiac cells.

In one embodiment, the polypeptides of the invention comprise a fragment of a Dkk protein. In a preferred embodiment, the fragment comprises the C-terminal cysteine rich domain of a Dkk protein, e.g., those indicated in FIGS. 18 and 19. For example, a polypeptide may comprise about amino acids 155, 156, 157, 158, 159 or 160 to about amino acids 260, 262, 262, 263, 264, 265 or 266 of SEQ ID NO: 2. A polypeptide may comprise about amino acids 130, 135, 145, 150, 155, 160, 165, 170, 175, 180 or 185 to about amino acid 255 or 259 of SEQ ID NO: 4. Fragments of Dkk proteins may comprise at most about 200, 150, 125, 110, 100, 90, 80, 70, 60 or 50 amino acids. Other exemplary polypeptides of the invention are also set forth in the FIG. 20 and in the Examples.

In yet another embodiment, the invention provides polypeptides which are fusion polypeptides comprising sequences from two or more Wnt antagonist polypeptides (e.g., different types of Wnt antagonists or derived from different species). For example, a polypeptide can be a fusion between two different Dkk proteins, e.g., Dkk proteins from different species or different types of Dkk proteins. An exemplary protein is one having a C-terminal cysteine rich domain from Dkk1 or Dkk2 and an N-terminal domain from another Dkk protein. Other fusion polypeptides provided by the invention include polypeptides that are modified to increase their half-life, e.g., immunoglobulin fusion proteins. For example, a polypeptide of the invention may comprise a C-terminal cysteine rich domain of Dkk1 or Dkk2 fused to the constant region of an immunoglobulin. Other fusion proteins comprise a sequence that is used to detect and/or isolate them, e.g., a 6× His tag.

Polypeptides of the invention can be full length or portions of naturally occurring Wnt antagonist proteins. The polypeptides can also be homologs of naturally-occurring Wnt antagonist polypeptides, such as non-naturally-occurring polypeptides. Homologs may differ from naturally-occurring Wnt antagonist proteins or fragments thereof by one or more amino acid deletion, addition or substitution. The substitution can a conservative or non-conservative substitution. In certain embodiments, polypeptides differ in at most 2, 3, 5, 10, 15, 20, 25, 30, or 50 amino acids from a naturally-occurring Wnt antagonist protein or fragment thereof. Other homologs include polypeptides that are encoded by a nucleic acid that hybridizes, e.g., under stringent hybridization conditions, to a nucleic acid encoding a Wnt antagonist protein.

Polypeptides of the invention, such as homologs of Wnt antagonist proteins or fragments of Wnt antagonist proteins have at least one biological activity of a Wnt antagonist protein. Most preferred polypeptides stimulate the differentiation of stem cells into cardiac cells. Even more preferred polypeptides accelerate and/or enhance the differentiation of stem cell into cardiac cells. Other homologs are at least 2, 3, 5, 10, 20, 30, 50, 100, 500 or 1000 times more potent (i.e., accelerates and/or enhances cardiac differentiation) relative to a naturally-occurring Dkk protein. Acceleration may be by one, two or at least three days. Enhancement refers to the number of stem cells that will differentiate into cardiac cells. Enhancement may be by a factor of at least 2, 5, 10, 20, 50 or over 100. Other polypeptides stimulate the differentiation of stem cells into cells of the same lineage as cardiac cells, e.g., pancreatic or liver cells. Assays, such as those described in the Examples can be used to determine the capability of polypeptides to stimulate cell differentiation, e.g., into cardiac cells. The cells used for testing the differentiation stimulating potential of a polypeptide can be any type of stem cell that is capable of differentiating into the desired cell type, e.g., cardiac cells. For example, they can be embryonic or adult stem cells; somatic stem cells, e.g., SP cells or cells from uncommitted mesoderm, as further described herein.

A polypeptide of the invention may also comprise a signal sequence, such as to enable the polypeptide to be secreted from a cell, e.g., a mammalian cell, in which it is synthesized. The signal sequence can be from a Wnt antagonist protein, such as a Dkk protein, or from a different protein. Signal peptides from human Dkk1 and Dkk2 are shown in FIGS. 18 and 19. Signal peptides are known in the art and can be identified by analysis with signal sequence predicting algorithms.

Amino acid and nucleic acid sequences for exemplary polypeptides of the invention, including, but not limited to, Wnt antagonists and BMP proteins, may be obtained by one having ordinary skill in the art, based on the teachings herein, from publicly available databes, such as GenBank (http://www.ncbi.nlm.nih.gov/). Examples of accession numbers for some of the polypeptides discussed herein include: axin (AAC51624, XP_{—}128515, NM_{—}131503), axin2 (AAF22799, AAF22800), crescent (AAF70300, AAB61752), cerberus (AAC02430, BAC54274), Frzb (AAB51298, AAF27643), Tcf (P36402), Dishevelled (AAH32459, NP_{—}034221), N-cadherin (NP_{—}001783, XM_{—}109359, CAA69397), β-catenin (P35222, Q02248), BMP-3 (P22444), BMP-2 (CAB82007), and BMP-1 (XP_{—}127857, AAA513833).

Polypeptides of the invention can be produced recombinantly, e.g., in a prokaryotic or eukaryotic expression system or in an in vitro transcription and translation system, according to methods known in the art. In certain embodiments, Wnt antagonists for use in the compositions and methods described herein may be synthesized chemically, ribosomally in a cell free system, or ribosomally within a cell. Chemical synthesis of Wnt antagonist polypeptides may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full-length ligation product having a native peptide bond at the ligation site. Full-length ligation products are chemically identical to proteins produced by cell free synthesis. Full-length ligation products may be refolded and/or oxidized, as allowed, to form native disulfide-containing protein molecules. (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Schnolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

Another aspect of the invention relates to polypeptides derived from the full-length Wnt antagonist polypeptides of the invention. Isolated peptidyl portions of those polypeptides may be obtained by screening polypeptides recombinantly produced from the corresponding fragment of the nucleic acid encoding such polypeptides. In addition, fragments may be chemically synthesized using techniques known in the art such as conventional Merrifield solid phase f-Moc or t-Boc chemistry. For example, proteins may be arbitrarily divided into fragments of desired length with no overlap of the fragments, or may be divided into overlapping fragments of a desired length. The fragments may be produced (recombinantly or by chemical synthesis) and tested to identify those peptidyl fragments having a desired property, for example, the capability of functioning as a modulator of the polypeptides of the invention. In an illustrative embodiment, peptidyl portions of a protein of the invention may be tested for binding activity, as well as inhibitory ability, by expression as, for example, thioredoxin fusion proteins, each of which contains a discrete fragment of a protein of the invention (see, for example, U.S. Pat. Nos. 5,270,181 and 5,292,646; and PCT publication WO94/02502).

In another embodiment, truncated Wnt antagonist polypeptides may be prepared. Truncated polypeptides have from 1 to 20 or more amino acid residues removed from either or both the N- and C-termini. Such truncated polypeptides may prove more amenable to expression, purification or characterization than the full-length polypeptide. In addition, the use of truncated polypeptides may also identify stable and active domains of the full-length polypeptide.

It is also possible to modify the structure of the Wnt antagonist polypeptides of the invention for such purposes as enhancing therapeutic or prophylactic efficacy, or stability (e.g., ex vivo shelf life, resistance to proteolytic degradation in vivo, etc.). Such modified polypeptides, when designed to retain at least one activity of the naturally-occurring form of the protein, are considered “functional equivalents” of the polypeptides described in more detail herein. Such modified polypeptides may be produced, for instance, by amino acid substitution, deletion, or addition, which substitutions may consist in whole or part by conservative amino acid substitutions.

For instance, it is reasonable to expect that an isolated conservative amino acid substitution, such as replacement of a leucine with an isoleucine or valine, an aspartate with a glutamate, a threonine with a serine, will not have a major affect on the biological activity of the resulting molecule. Whether a change in the amino acid sequence of a polypeptide results in a functional homolog may be readily determined by assessing the ability of the variant polypeptide to produce a response similar to that of the wild-type protein. Polypeptides in which more than one replacement has taken place may readily be tested in the same manner.

This invention further contemplates a method of generating sets of combinatorial mutants of Wnt antagonist polypeptides of the invention, as well as truncation mutants, and variant sequences (e.g. homologs). The purpose of screening such combinatorial libraries is to generate, for example, homologs which may have a greater activity for inducing differentiation of stem cells into cardiac cells. Such homologs may be used in the development of therapeutics.

Likewise, mutagenesis may give rise to homologs which have intracellular half-lives dramatically different than the corresponding wild-type protein. For example, the altered protein may be rendered either more stable or less stable to proteolytic degradation or other cellular process which result in destruction of, or otherwise inactivation of the protein. Such homologs, and the genes which encode them, may be utilized to alter protein expression by modulating the half-life of the protein. As above, such proteins may be used for the development of therapeutics or treatment.

In a representative embodiment of this method, the amino acid sequences for a population of protein homologs are aligned, preferably to promote the highest homology possible. Such a population of variants may include, for example, homologs from one or more species, or homologs from the same species but which differ due to mutation. Amino acids which appear at each position of the aligned sequences are selected to create a degenerate set of combinatorial sequences. In certain embodiments, the combinatorial library is produced by way of a degenerate library of genes encoding a library of polypeptides which each include at least a portion of potential protein sequences. For instance, a mixture of synthetic oligonucleotides may be enzymatically ligated into gene sequences such that the degenerate set of potential nucleotide sequences are expressible as individual polypeptides, or alternatively, as a set of larger fusion proteins (e.g. for phage display).

There are many ways by which the library of potential homologs may be generated from a degenerate oligonucleotide sequence. Chemical synthesis of a degenerate gene sequence may be carried out in an automatic DNA synthesizer, and the synthetic genes may then be ligated into an appropriate vector for expression. One purpose of a degenerate set of genes is to provide, in one mixture, all of the sequences encoding the desired set of potential protein sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp. 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis may be utilized to generate a combinatorial library. For example, protein homologs may be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated forms of proteins that are bioactive.

A wide range of techniques are known in the art for screening gene products of combinatorial libraries made by point mutations and truncations, and for screening cDNA libraries for gene products having a certain property. Such techniques will be generally adaptable for rapid screening of the gene libraries generated by the combinatorial mutagenesis of protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high throughput analysis as necessary to screen large numbers of degenerate sequences created by combinatorial mutagenesis techniques.

The invention also provides derivatives of Wnt antagonist polypeptides or fragments thereof, such as chemically modified polypeptides and peptidomimetics. In an exemplary embodiment, the invention provides derivatives of Dkk polypeptides or fragments thereof. Peptidomimetics are compounds based on, or derived from, peptides and proteins. The peptidomimetics of the present invention typically can be obtained by structural modification of a known peptide sequences using unnatural amino acids, conformational restraints, isosteric replacement, and the like. The subject peptidomimetics constitute the continum of structural space between peptides and non-peptide synthetic structures; peptidomimetics may be useful, therefore, in delineating pharmacophores and in helping to translate peptides into nonpeptide compounds with the activity of the parent peptides.

Moreover, as is apparent from the present disclosure, mimetopes of the subject polypeptides can be provided. Such peptidomimetics can have such attributes as being non-hydrolyzable (e.g., increased stability against proteases or other physiological conditions which degrade the corresponding peptide), increased specificity and/or potency for stimulating cell differentiation. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biologyy, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Communl26:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in in Peptides: Chemistry and Biology, G. R. Marshall ed., ESCOM Publisher: Leiden, Netherlands, 1988)

In addition to a variety of sidechain replacements which can be carried out to generate the subject peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

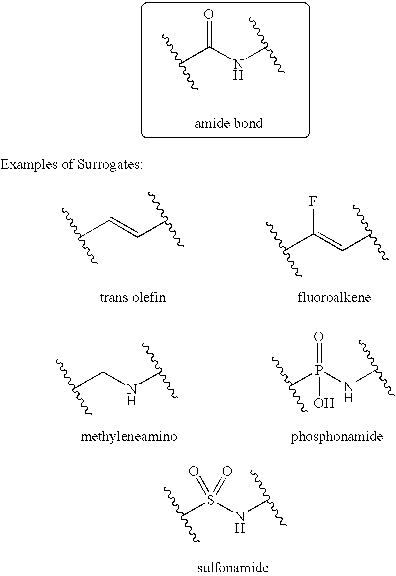

Additionally, peptidomimietics based on more substantial modifications of the backbone of a peptide can be used. Peptidomimetics which fall in this category include (i) retro-inverso analogs, and (ii) N-alkyl glycine analogs (so-called peptoids).

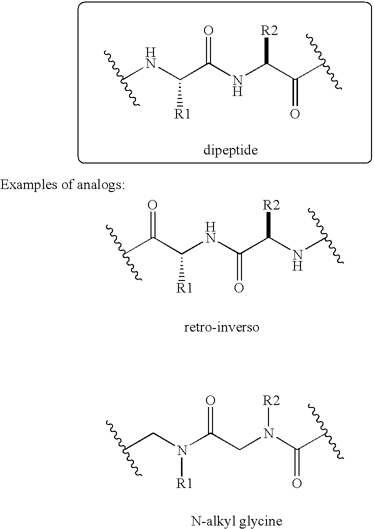

Furthermore, the methods of combinatorial chemistry are being brought to bear, e.g., by G. L. Verdine at Harvard University, on the development of new peptidomimetics. For example, one embodiment of a so-called “peptide morphing” strategy focuses on the random generation of a library of peptide analogs that comprise a wide range of peptide bond substitutes.

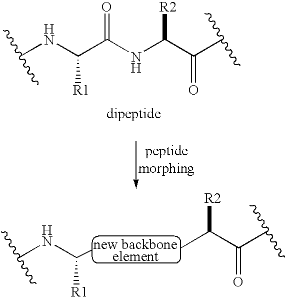

In an exemplary embodiment, the peptidomimetic can be derived as a retro-inverso analog of the peptide. Such retro-inverso analogs can be made according to the methods known in the art, such as that described by the Sisto et al. U.S. Pat. No. 4,522,752. A retro-inverso analog can be generated as described, e.g., in WO 00/01720. It will be understood that a mixed peptide, e.g. including some normal peptide linkages, may be generated. As a general guide, sites which are most susceptible to proteolysis are typically altered, with less susceptible amide linkages being optional for mimetic switching. The final product, or intermediates thereof, can be purified by HPLC.

In another illustrative embodiment, the peptidomimetic can be derived as a retro-enatio analog of a peptide. Retro-enantio analogs such as this can be synthesized commercially available D-amino acids (or analogs thereof) and standard solid- or solution-phase peptide-synthesis techniques, as described, e.g., in WO 00/01720. The final product may be purified by HPLC to yield the pure retro-enantio analog.

In still another illustrative embodiment, trans-olefin derivatives can be made for the subject polypeptide. Trans olefin analogs can be synthesized according to the method of Y. K. Shue et al. (1987) Tetrahedron Letters 28:3225 and as described in WO 00/01720. It is further possible to couple pseudodipeptides synthesized by the above method to other pseudodipeptides, to make peptide analogs with several olefinic functionalities in place of amide functionalities.

Still another class of peptidomimetic derivatives include the phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the subject peptidomimetics. To illustrate, the E2 peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject peptidomimetics can be optimized by, e.g., combinatorial synthesis techniques combined with high throughput screening.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of stimulating differentiation of stem cells into the desired cell type. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling. The predicted mimetope structures can then be produced by, for example, chemical synthesis, recombinant DNA technology, or by isolating a mimetope from a natural source (e.g., plants, animals, bacteria and fungi).

Nucleic acids encoding a polypeptide of the invention are also within the scope of the invention. A nucleic acid encoding a polypeptide of the invention may be linked to one or more transcriptional regulatory elements, e.g., a promoter and optionally enhancer. The nucleic acid may be in an expression vector, e.g., a prokaryotic expression vector or a eukaryotic expression vector. Eukaryotic expression vectors can be used, e.g., for gene therapy purposes, e.g., to treat cardiac failures. Also within the scope of the invention are host cells comprising a nucleic acid of the invention or a vector comprising such. Host cells may be prokaryotic or eukaryotic host cells, such as mammalian, e.g., human or non-human cells.

A nucleic acid encoding a Wnt antagonist polypeptide of the invention may be obtained from mRNA or genomic DNA from any organism in accordance with protocols described herein, as well as those generally known to those skilled in the art. A cDNA encoding a Wnt antagonist polypeptide, for example, may be obtained by isolating total mRNA from an organism, e.g. a vertebrate, mammal, etc. Double stranded cDNAs may then be prepared from the total mRNA, and subsequently inserted into a suitable plasmid or bacteriophage vector using any one of a number of known techniques. A gene encoding a Wnt antagonist polypeptide may also be cloned using established polymerase chain reaction techniques in accordance with the nucleotide sequence information provided by the invention. In one aspect, the present invention contemplates a method for amplification of a nucleic acid of the invention, or a fragment thereof, comprising: (a) providing a pair of single stranded oligonucleotides, each of which is at least eight nucleotides in length, complementary to sequences of a nucleic acid of the invention, and wherein the sequences to which the oligonucleotides are complementary are at least ten nucleotides apart; and (b) contacting the oligonucleotides with a sample comprising a nucleic acid comprising the nucleic acid of the invention under conditions which permit amplification of the region located between the pair of oligonucleotides, thereby amplifying the nucleic acid.

In another aspect of the invention, the subject nucleic acid is provided in an expression vector comprising a nucleotide sequence encoding a Wnt antagonist polypeptide of the invention and operably linked to at least one regulatory sequence. It should be understood that the design of the expression vector may depend on such factors as the choice of the host cell to be transformed and/or the type of protein desired to be expressed. The vector's copy number, the ability to control that copy number and the expression of any other protein encoded by the vector, such as antibiotic markers, should be considered.

The subject nucleic acids may be used to cause expression and over-expression of a Wnt antagonist polypeptide in cells propagated in culture, e.g. to produce proteins or polypeptides, including fusion proteins or polypeptides.

This invention pertains to a host cell transfected with a recombinant gene in order to express a Wnt antagonist polypeptide of the invention. The host cell may be any prokaryotic or eukaryotic cell. For example, a Wnt antagonist polypeptide of the invention may be expressed in bacterial cells, such as E. coli, insect cells (baculovirus), yeast, or mammalian cells. In those instances when the host cell is human, it may or may not be in a live subject. Other suitable host cells are known to those skilled in the art. Additionally, the host cell may be supplemented with tRNA molecules not typically found in the host so as to optimize expression of the polypeptide. Other methods suitable for maximizing expression of the polypeptide will be known to those in the art.

The present invention further pertains to methods of producing Wnt antagonist polypeptides. For example, a host cell transfected with an expression vector encoding a Wnt antagonist polypeptide may be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated.

A cell culture includes host cells, media and other byproducts. Suitable media for cell culture are well known in the art. The polypeptide may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of a polypeptide of the invention.

Thus, a nucleotide sequence encoding all or a selected portion of Wnt antagonist polypeptide, may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant polypeptides of the invention by microbial means or tissue-culture technology.

Expression vehicles for production of a recombinant protein include plasmids and other vectors. For instance, suitable vectors for the expression of a Wnt antagonist polypeptide of the invention include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83). These vectors may replicate in E. coli due the presence of the pBR322 ori, and in S. cerevisiae due to the replication determinant of the yeast 2 micron plasmid. In addition, drug resistance markers such as ampicillin may be used.

In certain embodiments, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant protein by the use of a baculovirus expression system. Examples of such baculovirus expression systems include pVL-derived vectors (such as pVL1392, pVL1393 and pVL941), pAcUW-derived vectors (such as pAcUWI), and pBlueBac-derived vectors (such as the B-gal containing pBlueBac III).

In another variation, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. In vitro transcription and translation may be coupled in a one-pot reaction to produce proteins from one or more isolated DNAs.

When expression of a carboxy terminal fragment of a polypeptide is desired, i.e. a truncation mutant, it may be necessary to add a start codon (ATG) to the oligonucleotide fragment containing the desired sequence to be expressed. It is well known in the art that a methionine at the N-terminal position may be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, may be achieved either in vivo by expressing such recombinant polypeptides in a host which produces MAP (e.g., E. coli or CM89 or S. cerevisiae), or in vitro by use of purified MAP (e.g., procedure of Miller et al.).

Coding sequences for a Wnt antagonist polypeptide of interest may be incorporated as a part of a fusion gene including a nucleotide sequence encoding a different polypeptide. The present invention contemplates an isolated nucleic acid comprising a Wnt antagonist nucleic acid and at least one heterologous sequence encoding a heterologous peptide linked in frame to the nucleotide sequence of the Wnt antagonist nucleic acid so as to encode a fusion protein comprising the heterologous polypeptide. The heterologous polypeptide may be fused to (a) the C-terminus of the polypeptide encoded by the nucleic acid of the invention, (b) the N-terminus of the polypeptide, or (c) the C-terminus and the N-terminus of the polypeptide. In certain instances, the heterologous sequence encodes a polypeptide permitting the detection, isolation, solubilization and/or stabilization of the polypeptide to which it is fused. In still other embodiments, the heterologous sequence encodes a polypeptide selected from the group consisting of a polyHis tag, myc, HA, GST, protein A, protein G, calmodulin-binding peptide, thioredoxin, maltose-binding protein, poly arginine, poly His-Asp, FLAG, a portion of an immunoglobulin protein, and a transcytosis peptide.

Fusion expression systems can be useful when it is desirable to produce an immunogenic fragment of a Wnt antagonist polypeptide. For example, the VP6 capsid protein of rotavirus may be used as an immunologic carrier protein for portions of polypeptide, either in the monomeric form or in the form of a viral particle. The nucleic acid sequences corresponding to the portion of a Wnt antagonist polypeptide to which antibodies are to be raised may be incorporated into a fusion gene construct which includes coding sequences for a late vaccinia virus structural protein to produce a set of recombinant viruses expressing fusion proteins comprising a portion of the protein as part of the virion. The Hepatitis B surface antigen may also be utilized in this role as well. Similarly, chimeric constructs coding for fusion proteins containing a portion of a polypeptide of the invention and the poliovirus capsid protein may be created to enhance immunogenicity (see, for example, EP Publication NO: 0259149; and Evans et al., (1989) Nature 339:385; Huang et al., (1988) J. Virol 62:3855; and Schlienger et al., (1992) J. Virol. 66:2).

Fusion proteins may facilitate the expression and/or purification of proteins. For example, a Wnt antagonist polypeptide may be generated as a glutathione-S-transferase (GST) fusion protein. Such GST fusion proteins may be used to simplify purification of a polypeptide of the invention, such as through the use of glutathione-derivatized matrices (see, for example, Current Protocols in Molecular Biology, eds. Ausubel et al., (N.Y.: John Wiley & Sons, 1991)). In another embodiment, a fusion gene coding for a purification leader sequence, such as a poly-(His)/enterokinase cleavage site sequence at the N-terminus of the desired portion of the recombinant protein, may allow purification of the expressed fusion protein by affinity -chromatography using a Ni²⁺ metal resin. The purification leader sequence may then be subsequently removed by treatment with enterokinase to provide the purified protein (e.g., see Hochuli et al., (1987) J. Chromatography 411:177; and Janknecht et al., PNAS USA 88:8972). Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992).

Biological Assays

The capability of stimulating differentiation of stem cells into a desired cell type, e.g., cardiac cells, can be monitored in biological assays. For example, a population of cells comprising stem cells is incubated in the presence of a Wnt antagonist polypeptide, or fragment, or homolog, or peptidomimetic thereof, and differentiation is monitored. In an exemplary embodiment the Wnt antagonist is a Dkk polypeptide, or fragment, or homolog, or peptidomimetic thereof (“Dkk reagent”). Differentiation in the presence of the Wnt antagonist may be compared to differentiation in the absence of it. The cells used for testing the differentiation stimulating potential of a polypeptide can be any type of stem cell that is capable of differentiating into the desired cell type, e.g., cardiac cells. For example, they can be embryonic or adult stem cells; somatic stem cells, e.g., SP cells or cells from uncommitted mesoderm, as further described herein.

In certain embodiments, the invention provides a method for inducing differentiation of stem cells into cardiac cells, comprising contacting a population of cells comprising stem cells with a sufficient amount of at least one Wnt antagonist to stimulate differentiation of at least a portion of the stem cells into cardiac cells, consistent with the results as presented in the Examples.

Differentiation of cells can be monitored by visual inspection or by monitoring the expression of markers of particular differentiation stages. Marker protein expression can be examined immunohistochemically or by RT-PCR. For example, differentiated cardiac cells can be identified by the presence of myosin light chain (MLC2a) (see Examples). Early markers of cardiac differentiation include Nkx2.5; GATA4, 5, 6; Tbx5; eHAND; and dHAND. Criteria for terminal cardiomyocyte differentiation include expression of genes encoding contractile proteins, e.g., myosin heavy chain (MF-20), troponin T (CT-3). Terminal differentiation can be assessed further by formation of sarcomeric arrays visible by confocal fluorescence microscopy (BioRad Radiance 2000) after staining with CT-3 or anti-descmin (DAKO) antibodies. Integration into myocardial tissue can be determined by visualization of gap and adherens junction proteins by staining with anti-Cx43 (MAB3068, Chemicon) and anti-pan-cadherin (CH-19, Sigma). Topologically normal patterns of anti-cadeherin and anti-Cx43 immunostaining at the ends of donor-derived cells suggests formation of intercalated disks characteristic of myocardial tissue. Evidence of sarcomeric array and intercalated disk formation is evidence of terminal differentiation and electromechanical coupling typical of myocardium.

Methods of the Invention

The invention provides methods for obtaining differentiated cells, e.g., cardiac cells. In one embodiment, the invention comprises contacting a population of cells comprising stem cells with a Wnt antagonist, e.g., a Wnt antagonist protein or fragment thereof, in amounts sufficient to stimulate the differentiation of at least a portion of the stem cells into differentiated cells of the mesodermal lineage, e.g., cardiac cells (e.g., cardiomyocytes), pancreatic and liver cells. In an exemplary embodiment, the Wnt antagonist is a Dkk polypeptide or a fragment thereof. In other embodiments, the stem cells are modified, e.g., by transfection, to contain a nucleic acid encoding a Wnt antagonist, such that the cells express the Wnt antagonist, which may be secreted. The stem cells can be embryonic stem (ES) cells, e.g., human and murine ES cells. The stem cells can be SP cells, e.g., derived from differentiated tissue (see below).

In certain embodiments, the present invention provides methods for obtaining differentiated cells comprising contacting a population of cells with a Wnt antagonist, e.g., a Wnt antagonist protein or fragment thereof, in amounts sufficient to stimulate the differentiation of at least a portion of cells into differentiated cells. In various embodiments, the cells that differentiate into cardiac cells may be stem cells, cardiac precursor cells, cardiac progenitor cells, or cells from a later stage of differentiation. In certain embodiments, exposure to a Wnt antagonist may stimulate cells to undergo transdifferentiation whereby cells are induced to change lineage commitment.

Accordingly, the invention also provides isolated populations of differentiated cells, such as cardiac cells, e.g., cardiomyocytes. In a preferred embodiment, the population of cells comprises at least about 70%, 80%, 90%, 95%, 98%, or 99% of differentiated cells. In another embodiment, the population of cells forms an embryonic heart. Populations of cells may also comprise less than about 20%, 15%, 10%, 5%, 2%, 1% of 0.1% of cells from a different lineage or of stem cells. The differentiated cells may be characterized by the presence of one or more markers described herein.

The invention also provides methods for identifying compounds (natural or synthetic) that modulate, e.g., stimulate or inhibit, differentiation of stem cells into the desired cell type. In one embodiment, a Wnt antagonist, such as a Dkk polypeptide, is used as a positive control. Compounds, e.g., factors, can be isolated, e.g., from tissue, such as differentiated heart tissue or embryonic tissue. Such assays can be conducted with tissue from a different species as that of the stem cells. A tissue can be, e.g., chick anterior mesoderm, or mesoderm or endoderm thereof. Alternatively, chemical libraries can be screened. Screens can be performed in multi-well plates, e.g., 384 well plates. High throughput screens of ES cells can be performed as described in the examples. For example, cardiomyocyte differentiation of ES cells is expected to occur in either 8-9 days in the presence of Dkk1 or in 13-14 days in the absence of Dkk1. Compounds that stimulate differentiation in less than 13-14 days are considered to stimulate the differentiation of stem cells into cardiac cells.

In one embodiment, ES cells in which LacZ is expressed from either the Nkx2.5 or the MHC alpha promoters are used (Tanaka et al. (1999) Development 126(7):1439). LacZ is preferably “knocked-in” the genome of the cells, replacing the endogenous Nkx2.5 or MHC alpha coding sequence. Expression of these genes marks commitment to the cardiac lineage and cardiomyocyte differentiation, respectively. Detecting lacZ is a convenient assay that can be performed in a 384 well format. LacZ can be detected using commercially available fluorescent (Molecular Probes) or luminescent substrates (Applied Biosystems). Confirmation of differentiation may be by detection of expression of Nkx2.5, GATA4, MHCalpha, cTN-1, MLC2a and desmin PCR using a Roche Lightcycler real-time PCR machine.

In one embodiment, cells are plated in multi-well plates. Compounds are deposited in each well using an automated plate filler and processed for LacZ determination in duplicate after various culture times, ranging between 7 and 14 days. As described herein, cardiogenesis in the absence of inducer is expected to occur at about 11-13 days, whereas cardiogenesis in the presence of an inducer should occur at 8-9 days and far more robustly (see Examples). To optimze compound concentration, pilot screens of about 3,000 wells each can be done using concentrations in the 1-20 μM range.

Secondary screens may be conducted to examine toxicity and expression of cardiac and non-cardiac markers. For example, to be of interest, a compound identified due to its effect on lacZ under control of Nkx2.5 should regulate expression of endogenous Nkx2.5 and not just enhance beta-galactosidase activity. Moreover, effects on a panel of non-cardiac genes can provide a primary evaluatin of specificity. Gene-based analyses can also be used to reveal whether a hit affects the cardiogenic program or only a subset of genes. For instance, a compound may elevate Nkx2.5, but not contractile protein genes, or vice-versa. Toxicity effects may be evaluated using a luminescent assay (CytoLucx, Perkin Elmer).

Lead compounds may then be evaluated on SP cells and on embryonic tissues. The effects of the compounds may also be evaluated on Xenopus and zebrafish embryos, and embryonic tissues. Embryos and embryonic tissues allow the compound's effect on complete cardiac development to be tested. Moreover, embryos also offer a stringent test of specificity because effects on other differentiating tissues can be examined.

Other cells that can be used in these assays include Side Population (SP) cells that are enriched for multipotent somatic cells. Fluorescence-activated cell sorting (FACS) based on low retention of Hoechst 33342 greatly enriches for a population of multipotent somatic cells. These cells, known as SP cells, comprise a minor population of weakly fluorescing cells distinct from the main population of highly fluorescent cells. Low retention of Hoechst 33342 is due to a verapamil-sensitive channel that might be the ABCG2 transporter. SP cells have been purified from bone marrow, skeletal muscle, cardiac muscle and other murine, human, porcine and avian tissues. SP cells express the stem cell antigen Sca-1 and have been shown to be multipotent upon re-introduction into mice, usually by injection into the tail vein of irradiated animals. SP cells lack the CD34 antigen, but they become CD34+ upon differentiation along hematopoietic lineages. SP cell populations have been shown to contain Nkx2.5 positive cells.

SP cells can be isolated from tissues harvested from E10-12 day-old quails or 6-8 week old mice. Other ages can also be used. Bone marrow can be extracted from femurs and tibias. Primary skeletal and cardiac muscle cells can be isolated from tissue samples from donors. Dissected limb or cardiac muscle can be dissociated by mincing followed by digestion with dispase-II and collagenase-D. The cells can then be filtered to remove debris and red blood cells can be lysed with ammonium chloride.

FACS isolation of SP cells relies on Hoechst 33342 and propidium iodide to distinguish different cell populations. Cells can be incubated in Hoechst 33342 at 37° C. for 60-90 minutes. Cells can then be collected by centrifugation, washed in PBS and resuspended in a propidium iodide solution. As a negative control, a fraction of the primary cells are incubated in parallel with verapamil, which blocks Hoechst 33342 efflux. Sorting can be performed on a FACS Advantage Plus flow cytometer and fluorescence of Hoechst 33342 and propidium iodide are measured on a linear scale (Goodell et al. (1996) J. Exp. Med. 183:1797; Goodell et al. (1997) Nat. Med. 3:1337 and Gussoni et al. (1999) Nature 401:390).

Quail or mouse SP cells can be pelleted in a microfuge and the pellets can be manually divided under a dissecting microscope into small, loose aggregates. Compound of interest can then be added. When identifying factors from tissue, aggregates of SP cells may be positioned onto a sheet of dissected chick (or other) tissue on a Millipore filter floating in alpha MEM+20% FCS. Candidate inducing tissues include stage 5-6 anterior mesendoderm (staging (HH) is according to Hamburger and Hamilton, 1951) which forms heart tissue because of signals provided by the endoderm. HH stage 5-6 is when heart induction occurs in the embryo. Endoderm and mesoderm alone can also be examined, as well as neural, kidney and other inducing tissues.

Stem cells suitable for use in accordance with the methods described herein may be harvested from a patient, or from other sources, including, but not limited to, donor SP cells, human ES cells, human adult stem cells, human ES cells from an established or new cell line, and non-human (e.g., pig, etc.) sources. When using cells that have been derived from a heterologous source (e.g., not from the patient that is being treated), it may be desirable to modify the cells to reduce their immunogenicity and decrease host-rejection, or to administer the cells in conjunction with therapeutics that reduce or prevent transplant rejection.

When testing isolated compounds, e.g., polypeptides, these can be either added to media or delivered from protein-soaked resin beads implanted in tissues (Zhu et al. (1999) Curr. Biol. 9: 931).

The methods of the invention for differentiating stem cells may further include an inhibitor of LRP6.

In yet another embodiment, the invention provides methods for identifying compounds that modulate the interaction between LRP6 and a C-terminal cysteine rich domain of a Dkk protein. The method may comprise contacting an LRP6 protein, isolated or linked to a membrane, with a C-terminal cysteine-rich domain of a Dkk protein and monitoring the interaction in the presence relative to the absence of a test compound.

Uses

The invention can be used to produce cardiac cells, such as cardiomyocytes. These cells can be used for a variety of therapeutic applications, including transplantation for replacement of dead or damaged cardiac tissue. Myocardial damage, such as after an infarct, leads to apoptotic and necrotic cardiomyocytes that are eventually replaced by fibroblasts to form scar tissue, resulting in regional contractile dysfunction. Endogenous regeneration is clinically negligible, in part because adult cardiomyocytes respond to mitogenic signals by cellular hypertrophy rather than by cell division. Extensive efforts have been directed towards identifying cells that can be transplanted into injured myocardium to prevent heart failure. Over the past decade, grafts of fetal cardiomyocytes have been noted to integrate into infarcted myocardium, in species ranging from dogs and rodents to pigs (see, e.g., Koh et al. (1995) J. Clin. Invest. 96:2034 and Scorsin et al. (2000) J. Thorac. Cardiovasc. Surg. 119:1169). In certain circumstances, stable integration of -grafted fetal cardiomyocytes improved post-infarction function, increased angiogenesis, and appeared coupled to host cardiomyocytes by adherens and gap junctions (indicative of electromechanical coupling). Accordingly, the cardiac cells of the invention can be used to treat cardiac failures.

One way to induce stem cells to differentiate into cardiac cells is to exogenously apply a Wnt antagonist to the cells. Alternatively, cells may be transfected with DNA sequences encoding one or more Wnt antagonists so that the cells produce Wnt antagonist proteins.

Potential uses of the Wnt antagonists of the present invention include use of the Wnt antagonists to treat patients with cardiac tissue damage or stress. For example, as an adjunct to surgical procedures, cultured cells which are capable of differentiation into cells of cardio- or cardiomyocyte lineage are implanted into the damaged or stressed tissue and the composition may be applied directly to damaged or stressed tissue. Cells that may be useful in this and other applications of the present invention include stem cells, embryonic stem cells and side population cells. Cardiac cells as described herein may be used as part of a cell therapy by methods known in the art, including, but not limited to grafting, seeding, injection, etc.

Alternatively, the composition may be used to treat cells, whether autologous or heterologous, to promote the growth, proliferation, differentiation and/or maintenance of cells of a cardio- or cardiomyocyte lineage. The cells thus treated may then be applied to the damaged or stressed tissue, either alone or in conjunction with one or more Wnt antagonists of the present invention.

In another embodiment, DNA sequences encoding one or more Wnt antagonists may be transfected into cells, rendering the cells capable of producing the Wnt antagonist proteins. The transfected cells, which are capable of producing the Wnt antagonist proteins, may then be implanted at the site of damaged or stressed tissue.

An appropriate matrix may be used with any of the above embodiments in order to maintain the composition and/or cells at the site of damaged or stressed tissue. Alternatively, an injectable formulation of the composition may be used for administration of the compositions of protein and/or cells. The above may also be used for prophylactic measure in order to prevent or reduce damage or stress to tissue.

The dosage regimen for a particular application will be determined by the attending physician considering various factors which modify the action of the protein composition, e.g. amount of tissue desired to be formed, the site of tissue damage, the condition of the damaged tissue, the size of a wound, type of damaged tissue, the patient's age, sex, and diet, the severity of any infection, time of administration and other clinical factors. The dosage may vary with the type of stem cells used, the type of matrix used in the reconstitution and the types of Wnt antagonist proteins in the composition. The addition of other therapeutic factors, including growth factors such as a BMP, to the final composition, may also affect the dosage.

The method of the invention can also be used to identify modulators of cardiogenesis. Compounds identified by such methods as stimulators of cardiomyogenesis could be administered to subjects having cardiac failures. Similarly, the Wnt antagonists of the invention, such as Dkk reagents, can be administered to a subject having a cardiac failure, including, but not limited to, myocardial infarction and congestive heart failure.

Initiation of cardiogenesis in cultured cells can also be used to identify genetic markers of discrete steps in the cardiogenic program. Current knowledge of the cardiogenic program is limited to a few marker genes and additional genes are needed to identify and understand the effects of pharmacologic inducers of heart tissue.

The the Wnt antagonists of the invention, such as Dkk reagents, can also be used as a positive control in cell cultures induced to differentiate. For example, the Wnt antagonists can be used as a positive control in assays to identify compounds that modulate cardiac, liver or kidney cell differentiation.

The methods described herein also contemplate a method for stimulating the differentiation of stem cells into cardiac cells which further involves assessing the efficacy of the differentiation process before harvesting the cardiac cells. For example, such a method may involve contacting a population of cells comprising stem cells with with a sufficient amount of at least one Wnt antagonist to stimulate differentiation of the stem cells into cardiac cells and evaluating the efficacy of the differentiation process before utilizing said cardiac cells. Such a method may be beneficial if the process of differentiation does not occur in >50%, 75% or 90% of the cells in each differentiation process or at least in 50%, 75% or 90% of the differentiation processes. Such methods may also be accompanied by methods for isolating subpopulations of cells, such as cell sorting using FACS, to isolate the portion of the cells that have differentiated into cardiac cells from those that have not.

The invention also provides kits containing ingredients and/or reagents for differentiating stem cells into differentiated cells, e.g., cardiac cells.

The present invention also encompasses pharmaceutical compositions of a Wnt antagonist, or a pharmaceutically acceptable salt thereof, and a pharmaceutically acceptable carrier, adjuvant, or vehicle. The pharmaceutical compositions may comprise a Wnt antagonist, cells, and combinations thereof, and additionally may include other factors or therapeutic agents, including, but not limited to BMPs. The term “pharmaceutically acceptable carrier” refers to a carrier(s) that is “acceptable” in the sense of being compatible with the other ingredients of a composition and not deleterious to the recipient thereof.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. The term parenteral as used herein includes subcutaneous, intracutaneous, intravenous, intramuscular, intra articular, intrasynovial, intrasternal, intrathecal, intralesional, and intracranial injection or infusion techniques.

Dosage levels of between about 0.01 and about 100 mg/kg body weight per day, preferably between about 0.5 and about 75 mg/kg body weight per day of the modulators described herein are useful for the prevention and treatment of disease and conditions, including diseases and conditions mediated by pathogenic speices of origin for the polypeptides of the invention. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

The present invention is further illustrated by the following examples, which should not be construed as limiting in any way. The contents of all cited references including literature references, issued patents, published and non published patent applications as cited throughout this application are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

### Example 1

### Wnt Signals from the Neural Tube Block Ectopic Cardiogenesis

Prior studies have indicated that signals from the neural tube suppress heart formation in adjacent tissue (Jacobson 1960, 1961; Climent et al. 1995; Schultheiss et al. 1997; Raffin et al. 2000). Jacobson first noted that cardiogenesis in explants of precardiac tissue from newt embryos was significantly inhibited by the presence of the neural tube (Jacobson 1960, 1961).

In contrast, anterior endoderm has heart-inducing properties, as demonstrated by the ability of this tissue to promote heart formation in coculture with posterior primitive streak, a tissue normally fated to form blood (Schultheiss et al. 1995). Thus, extirpation of the endoderm blocked heart formation in gastrula-stage newt embryos, whereas extirpation of both the endoderm and neural plate restored heart formation (Jacobson 1960, 1961). This work suggests that the neural tube secretes a signal that inhibits cardiac differentiation in neighboring mesoderm.

In addition to a heart-promoting signal from the anterior endoderm, bone morphogenetic proteins (BMPs) expressed in lateral endoderm and ectoderm are also required for heart formation in chick embryos (Schultheiss et al. 1997; Schlange et al. 2000). Administration of BMP-2 induces cardiogenesis in explants of anterior medial mesendoderm from stage 6 chick embryos, as assayed by the expression of the cardiac regulators Nkx-2.5, GATA-4, GATA-5, GATA-6, MEF2, eHAND, and dHAND and the cardiac structural gene, ventricular myosin heavy chain (vMHC; Schultheiss et al. 1997; Schlange et al. 2000). However, when the adjacent neural tube and notochord was included in these explants, BMP-2 administration could only induce the expression of Nkx-2.5 and failed to induce the expression of either GATA-4 or vMHC (Schultheiss et al. 1997). Similarly, in vivo implantation of BMP-2-soaked beads between the neural plate and the anterior medial mesendoderm of stage 6 chick embryos induced robust ectopic expression of Nkx-2.5 but only trace levels of ectopic GATA-4 and no detectable ectopic vMHC (Schultheiss et al. 1997).

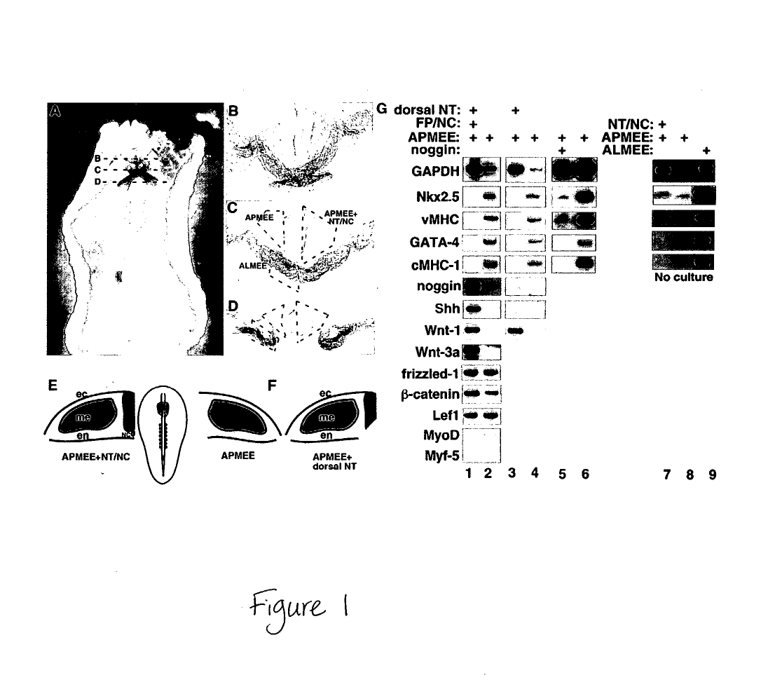

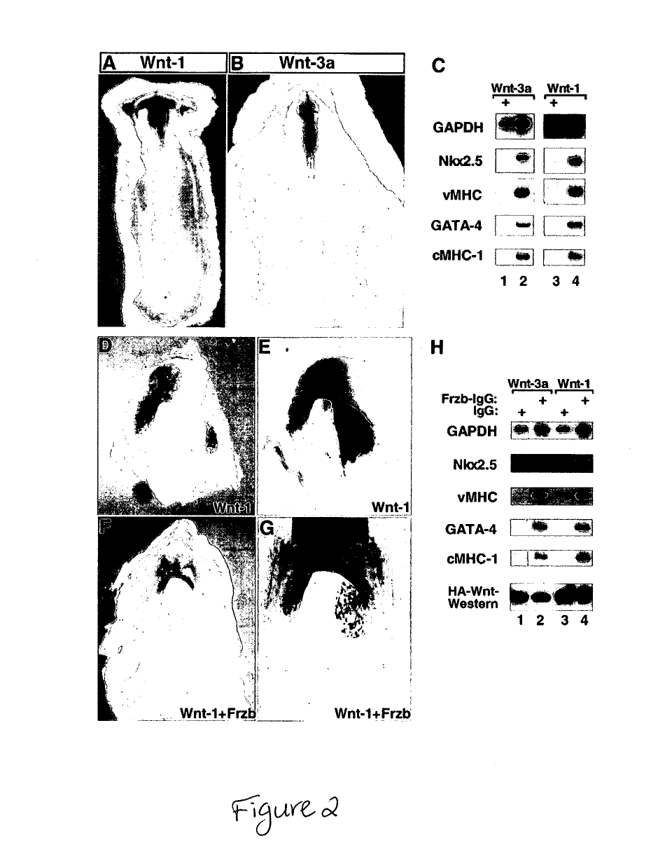

Because prior work has suggested that signals from the neural tube may block cardiogenesis, we sought to determine if signals from the neural tube also inhibit cardiogenesis in anterior paraxial mesendoderm in stage 9 chick embryos. FIG. 1, panels A-D, illustrates the relative positions of tissues employed in this study. While the ventrally located heart-forming mesoderm and pharyngeal endoderm both express Nkx-2.5, the more dorsal anterior paraxial mesoderm, which lies adjacent to the neural tube, does not express this gene (FIG. 1, panels A-D). We dissected anterior paraxial mesendoderm and ectoderm (APMEE; FIG. 1C) from stage 8-9 chick embryos and cultured this tissue either alone or in the presence of the adjacent neural tube and notochord (FIG. 1E). When cultured in the presence of the axial tissues, APMEE explants neither beat nor expressed the cardiac markers Nkx-2.5, GATA-4, vMHC, and cMHC-1 (FIG. 1G, lane 1). The latter gene is a chick myosin heavy-chain isoform expressed exclusively within the heart (Croissant et al. 2000). In contrast, when cultured in the absence of the neural tube and notochord, APMEE explants underwent cardiac differentiation, as evidenced by beating in ˜25% of such explants (n=80) and displayed robust expression of Nkx-2.5, GATA-4, vMHC, and cMHC-1 transcripts in nearly all such explants (FIG. 1G, lanes 2, 4, 6). Although anterior paraxial mesoderm is fated to give rise to both head mesenchyme and skeletal muscles (Christ and Ordahl 1995), the skeletal muscle regulators, MyoD and Myf-5, were not expressed in APMEE explants that expressed cardiac markers after 48 h culture in vitro (FIG. 1G, lane 2). Thus, after 48 h in culture, explanted APMEE gives rise to cardiac but not skeletal muscle tissue. At the time of dissection, explants of APMEE tissue expressed only trace levels of Nkx-2.5 and no detectable levels of GATA-4, vMHC, or cMHC-l (FIG. 1G, lanes 7, 8), whereas explants of anterior lateral mesendoderm plus ectoderm (ALMEE), which includes the heart-forming region, expressed abundent levels of these transcripts (FIG. 1G, lane 9). These findings imply that removal of the APMEE from the repressive influence of the axial tissues allowed this tissue to activate the cardiac myocyte-differentiation program in vitro.

To define the source of the repressive signal(s) that blocks cardiac myogenesis in APMEE tissue, we cultured APMEE explants with dorsolateral neural tube, lacking the floor plate and notochord (illustrated in FIG. 1F). Cardiogenesis was similarly inhibited in APMEE explants cocultured with the neural tube in either the presence or absence of the ventral midline tissues (FIG. 1G, lanes 1 and 3, respectively). Thus, signals from the dorsolateral neural tube arc sufficient to inhibit cardiogenesis in APMEE explants. Because removal of the ventral midline tissues eliminates the source of the BMP-antagonist, noggin, and Shh in these explants, these results suggest that other signals from the axial tissues repress heart formation. Nonetheless, administration of the BMP-antagonist noggin was sufficient to inhibit cardiogenesis in APMEE explants (FIG. 1G, lane 5), consistent with prior findings that heart formation requires BMP signaling (Schultheiss et al. 1997; Schlange et al. 2000). Thus, we conclude that in addition to noggin, which is expressed in the notochord, another signal expressed in the dorsal neural tube also blocks heart formation in APMEE tissue.

Wnt-1 and Wnt-3a are expressed in the open neural plate and dorsal neural tube adjacent to the anterior paraxial mesoderm (FIG. 2, panels A, B). These signaling molecules are highly expressed in explants containing both the APMEE and the neural tube but are not significantly expressed in AMPEE explants when cultured alone (FIG. 1G, lanes 1, 2). In addition to expression of Wnt family members in the neural tube, we detected expression of Frizzled-1, β-catenin, and Lef1, all of which are components of the Wnt signaling cascade, in APMEE explants (FIG. 1G, lanes 1, 2). Because Wnt-1 and Wnt-3a are expressed in the neural tube that lies adjacent to the AMPEE, we assayed whether these Wnt family members could mimic the inhibitory effects of the neural tube on cardiogenesis. Stage 9 APMEE explants were infected with avian retroviral vectors encoding either Wnt-3a (RCAS-Wnt-3a) or alkaline phosphatase (RCAS-AP) as a control (FIG. 2C, lanes 1, 2). Alternatively, Rat-i cells stably overexpressing Wnt-1 or parental Rat-1 fibroblasts were cocultured with APMEE explants (FIG. 2C, lanes 3, 4). In the absence of ectopic Wnt administration, these explants underwent full cardiac differentiation (FIG. 2C, lanes 2, 4). In contrast, APMEE explants exposed to either Wnt-3 a or Wnt-1 failed to activate expression of any cardiac markers (FIG. 2C, lanes 1, 3). In addition, implantation of fibroblasts expressing Wnt-1 into one side of the heart-forming region of stage 7 chick embryos blocked subsequent expression of Nkx-2.5 (FIG. 2, panels D, E). These findings indicate that Wnt signals are potent inhibitors of cardiogenesis both in vitro and in vivo.

Wnt signals are transduced by members of the Frizzled receptor family, which contains seven-transmembrane domains and an extracellular cysteine-rich domain (CRD) that interacts with the Wnt ligand (Bhanot et al. 1996). A family of soluble Frizzled-related secreted proteins (Sfrp; also known as Frzb/Sarp) share the Frizzled CRD domain but not the transmembrane domains and have been demonstrated to block Wnt signaling (Leyns et al. 1997; Rattner et al. 1997; Wang et al. 1997). We have fused one such chick Sfrp with the Fe region of IgG, to generate a reagent (termed Frzb-IgG) that blocks both Wnt-3a and Wnt-1 signaling (see below). Cardiogenesis was blocked in APMEE explants cocultured with fibroblasts expressing either Wnt-3a or Wnt-1 that had been transfected with the IgG expression vehicle (FIG. 2H, lanes 1, 3). In contrast, cardiogenesis took place in APMEE explants cocultured with Wnt-expressing fibroblasts that had been transiently transfected with an expression vehicle encoding Frzb-IgG (FIG. 2H, lanes 2, 4). Importantly, transfection of Frzb-IgG did not alter the levels of Wnt produced by the fibroblasts (FIG. 2H, lanes 1-4). Transfection of Frzb-IgG into Wnt-1-expressing fibroblasts similarly blocked the ability of these cells to extinguish Nkx-2.5 gene expression in vivo (FIG. 2, panels F, G). Thus, expression of the Frzb-IgG fusion is capable of blocking the ability of either Wnt-1 or Wnt-3a to inhibit cardiogenesis in APMEE tissue either in vitro or in vivo.

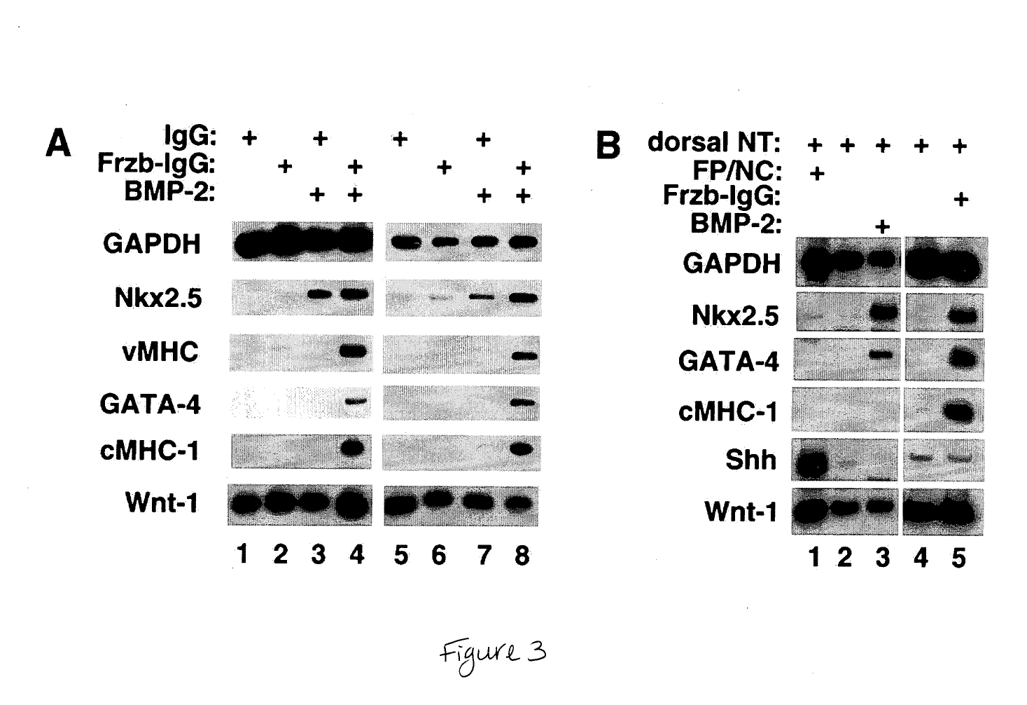

To address whether Wnt signals from the neural tube block cardiogenesis in the anterior paraxial mesendoderm, we cultured explants containing both APMEE and the neural tube and notochord (as shown schematically in FIG. 1E) with either control IgG, Frzb-IgG alone, BMP-2 alone, or the combination of Frzb-IgG and BMP-2. No cardiac markers were detected in explants exposed to either soluble IgG (FIG. 3A, lane 1) or to IgG expressing cells (FIG. 3A, lane 5). Addition of either soluble Frzb-IgG (FIG. 3A, lane 2) or Frzb-IgG-expressing cells (FIG. 3A, lane 6) to these cultures induced only trace levels of Nkx-2.5 yet failed to induce either GATA-4 or vMHC. Addition of BMP-2 alone induced higher levels of Nkx-2.5 but, similarly, failed to induce expression of either GATA-4 or vMHC (FIG. 3A, lanes 3, 7), consistent with previous findings (Schultheiss et al. 1997). In striking contrast, addition of the combination of either soluble Frzb-IgG- or Frzb-IgG-expressing cells plus BMP-2 induced expression of Nkx-2.5, GATA-4, vMHC, and cMHC-1 in cultures containing the APMEE and the axial tissues (FIG. 3A, lanes 4, 8). Cardiac gene expression was limited to the APMEE cells in these cultures, as neural tube cultured in the presence of Frzb-IgG plus BMP-2 failed to express any cardiac marker genes (data not shown). These findings indicate that signals from the axial tissues that block cardiogenesis in the anterior paraxial mesoderm can be reversed by the combination of a Wnt antagonist working in concert with BMP signals.

Although the dorsal neural tube expresses several BMP family members (Liem et al. 1995), we found that Frzb-IgG could only elicit cardiogenesis in APMEE cultured with the axial tissues in the presence of exogenous BMP-2. We speculated that the requirement of both exogenous BMP and Frzb-IgG to promote cardiogenesis in these cultures may be because of the expression of the BMP-antagonists, noggin, and chordin in the notochord. Therefore, we tested whether cardiogenesis in APMEE explants cultured solely with the dorsal neural tube could be elicited by administration of Frzb-IgG alone. Indeed, administration of Frzb-IgG to APMEE cultured with only the dorsal neural tube induced a robust cardiogenic response in the absence of exogenous BMP-2 (FIG. 3B, lane 5). In parallel cultures, BMP-2 administration induced GATA-4 and Nkx2.5 yet failed to elicit expression of cMHC-1 (FIG. 3B, lane 3). Thus, signals from the dorsal neural tube that suppress cardiogenesis in the adjacent APMEE can be completely reversed by administration of the Wnt antagonist Frzb-IgG.

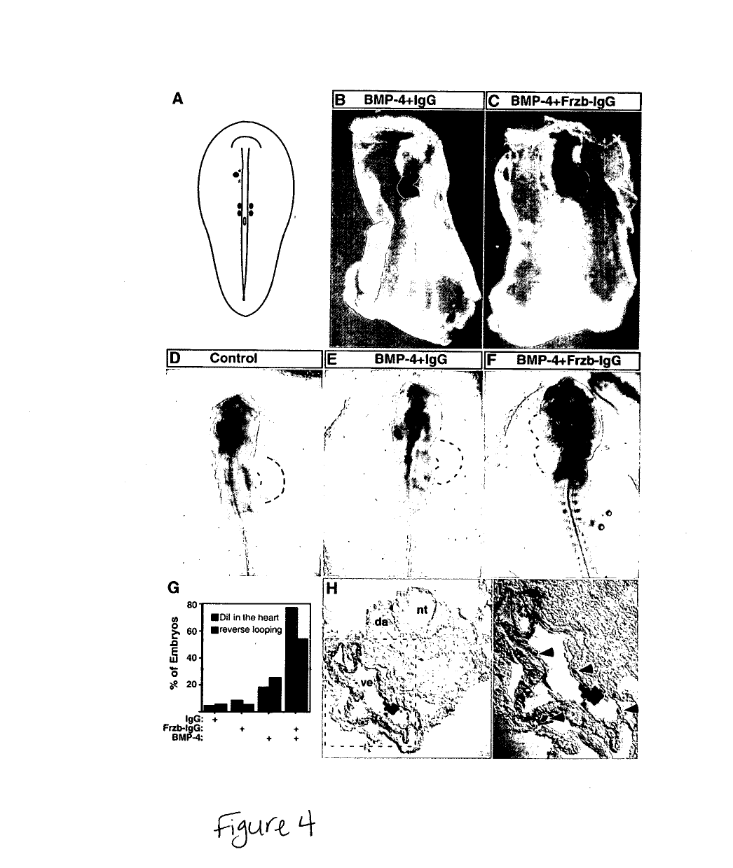

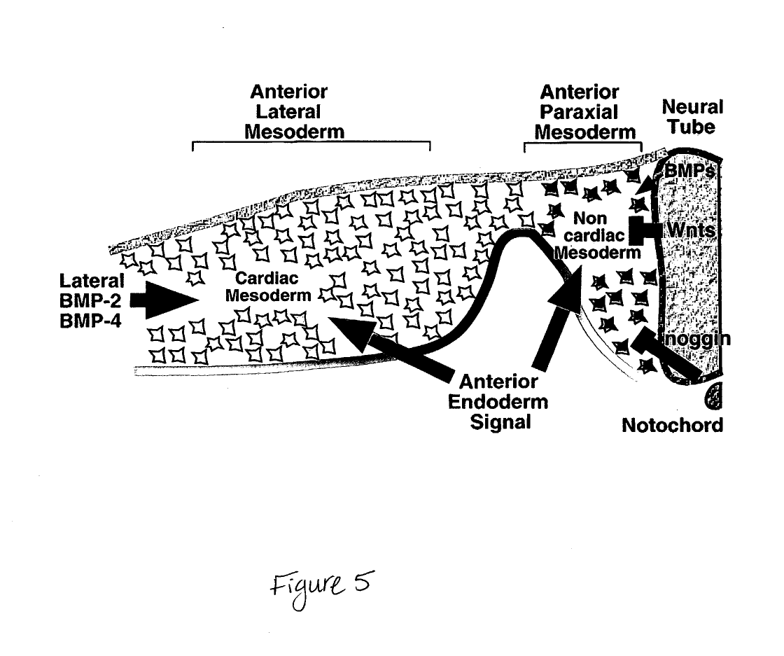

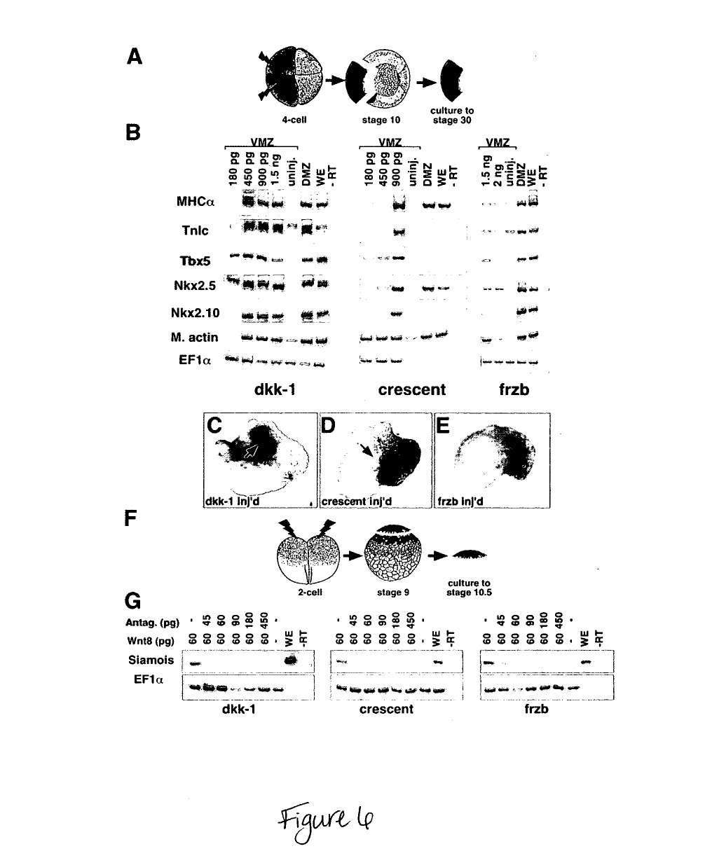

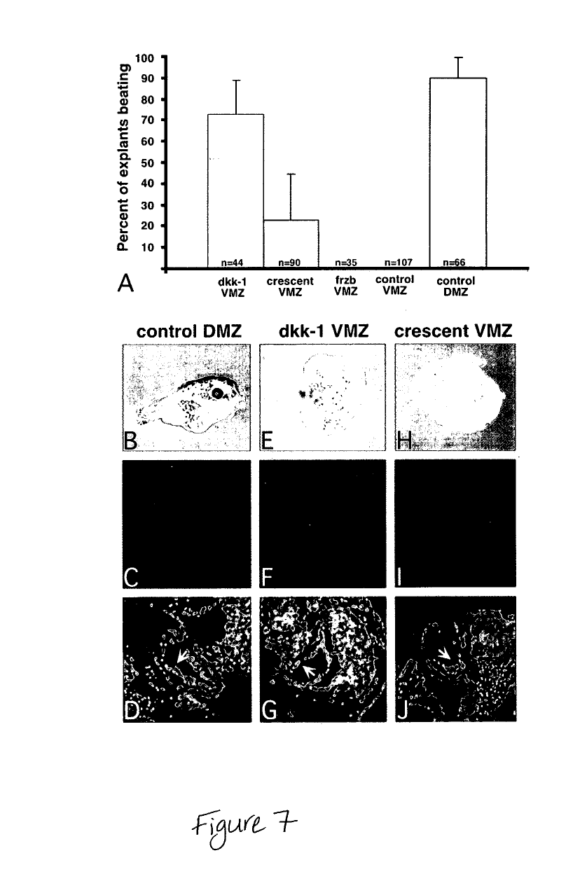

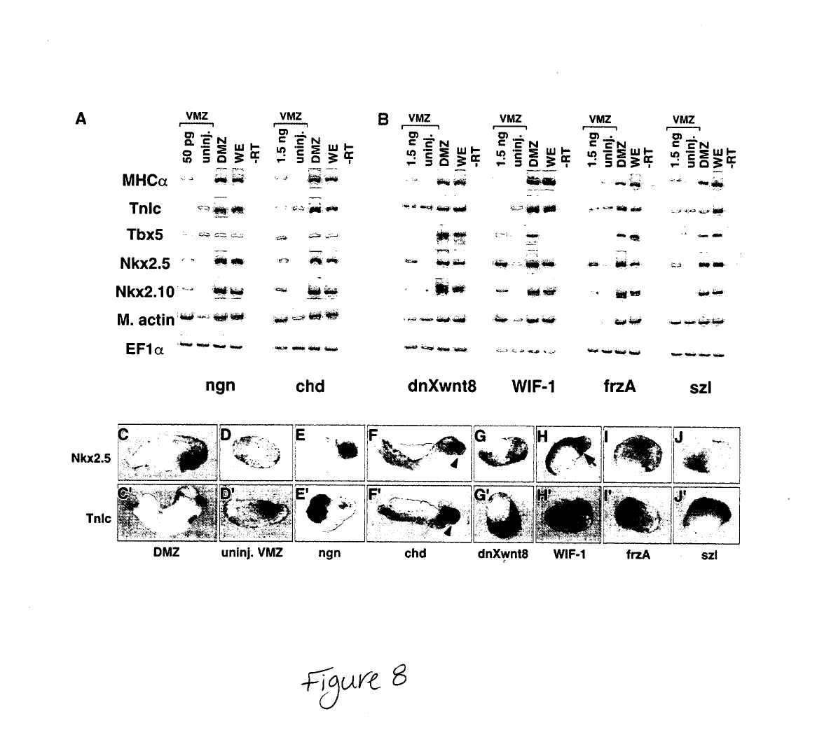

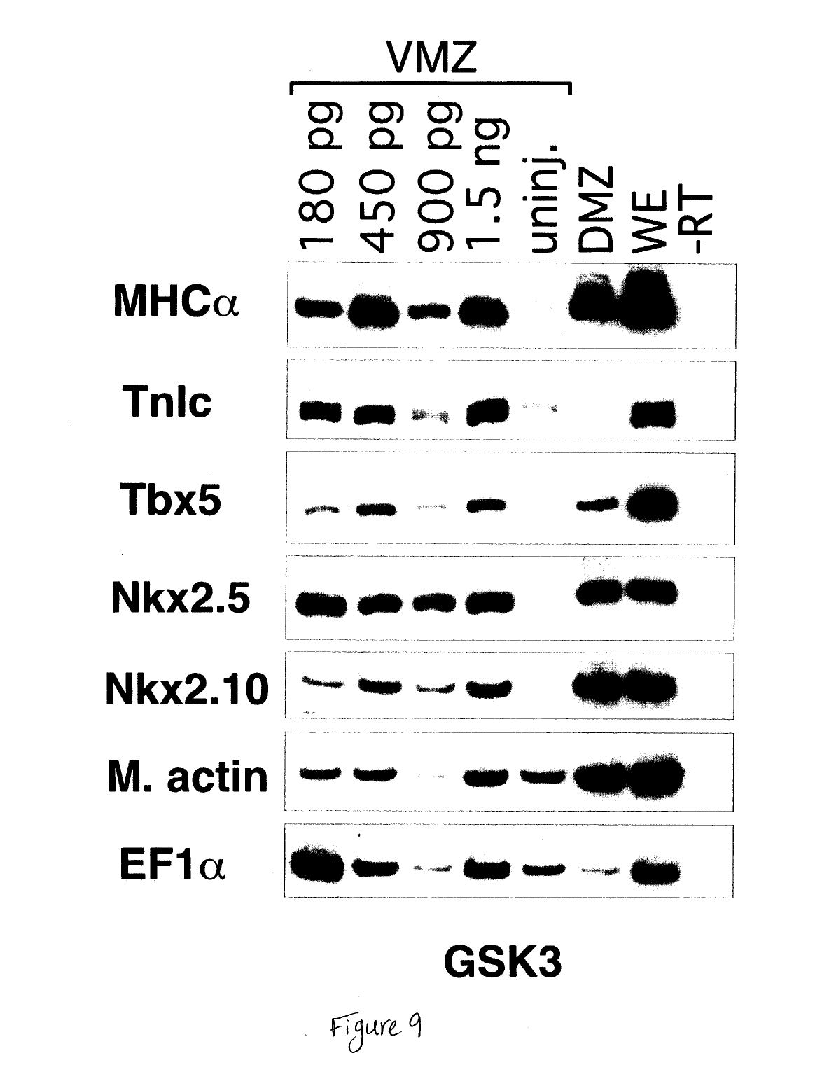

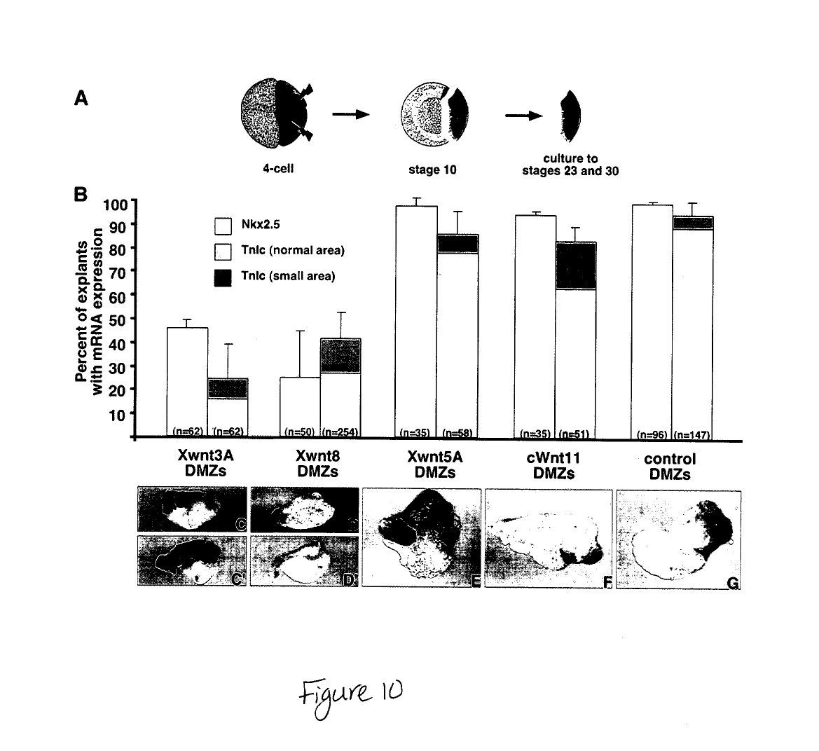

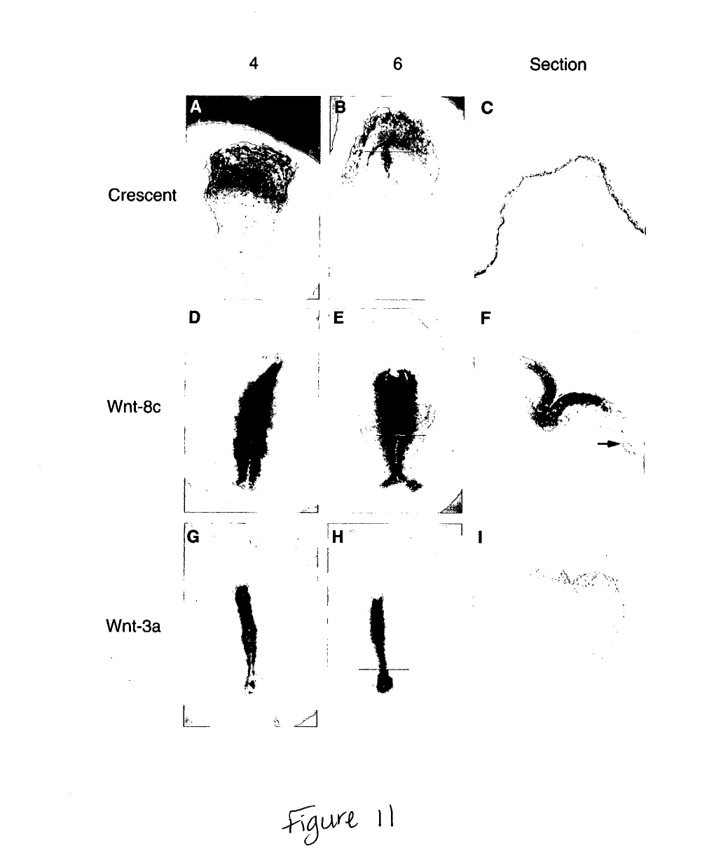

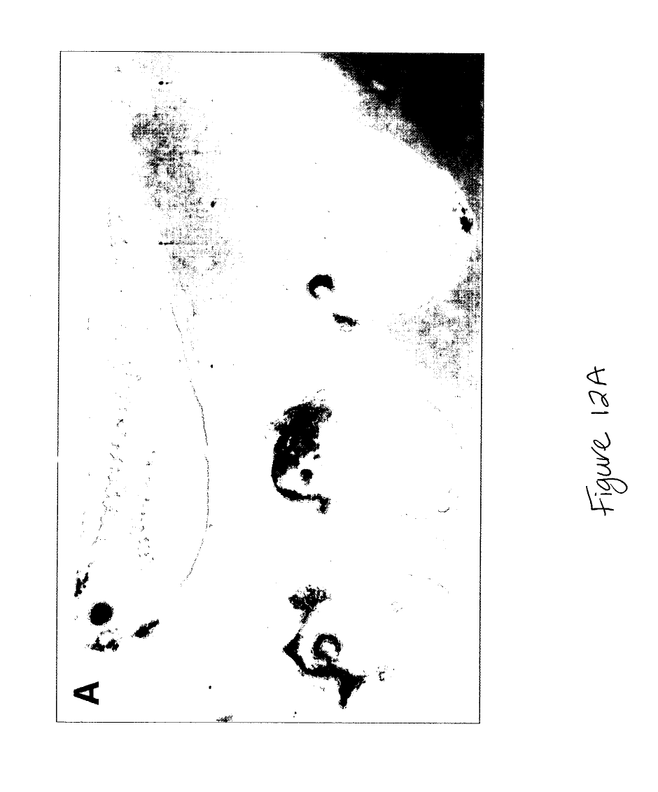

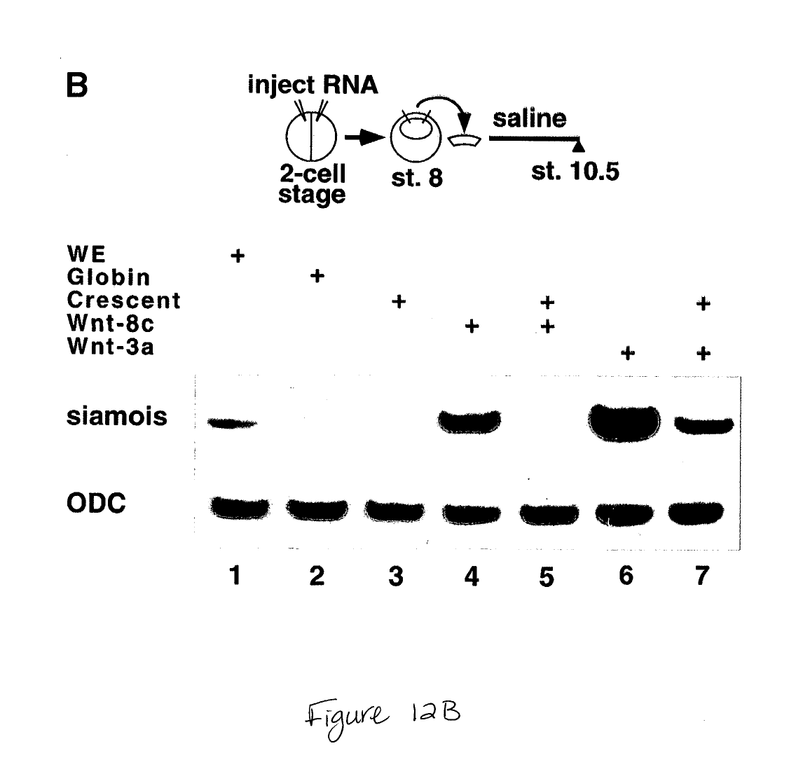

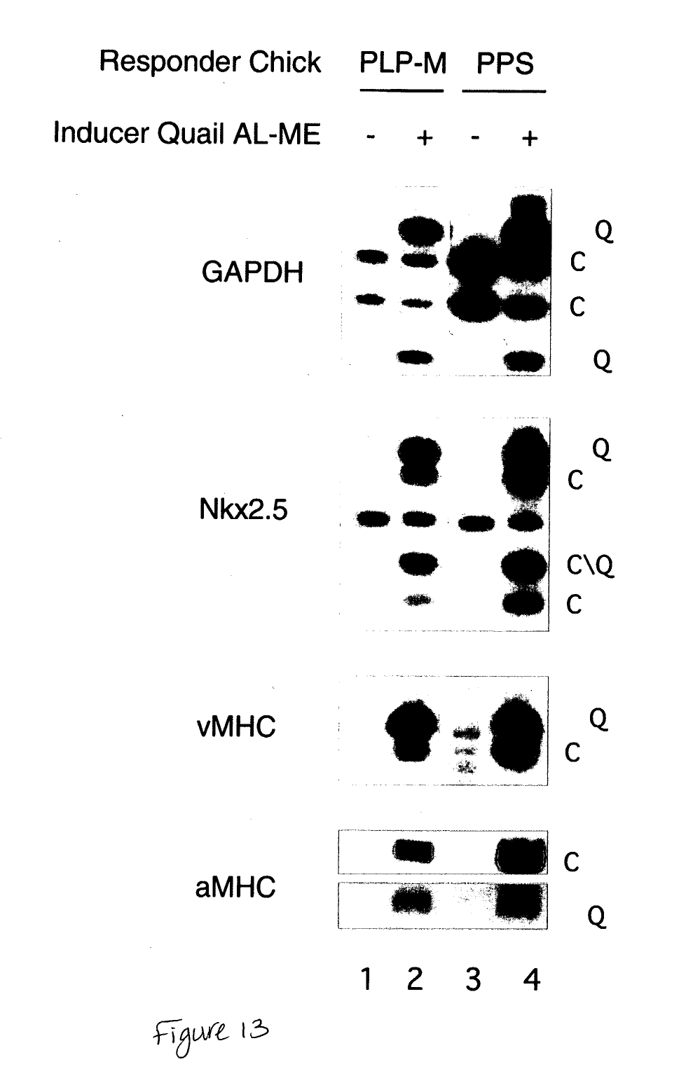

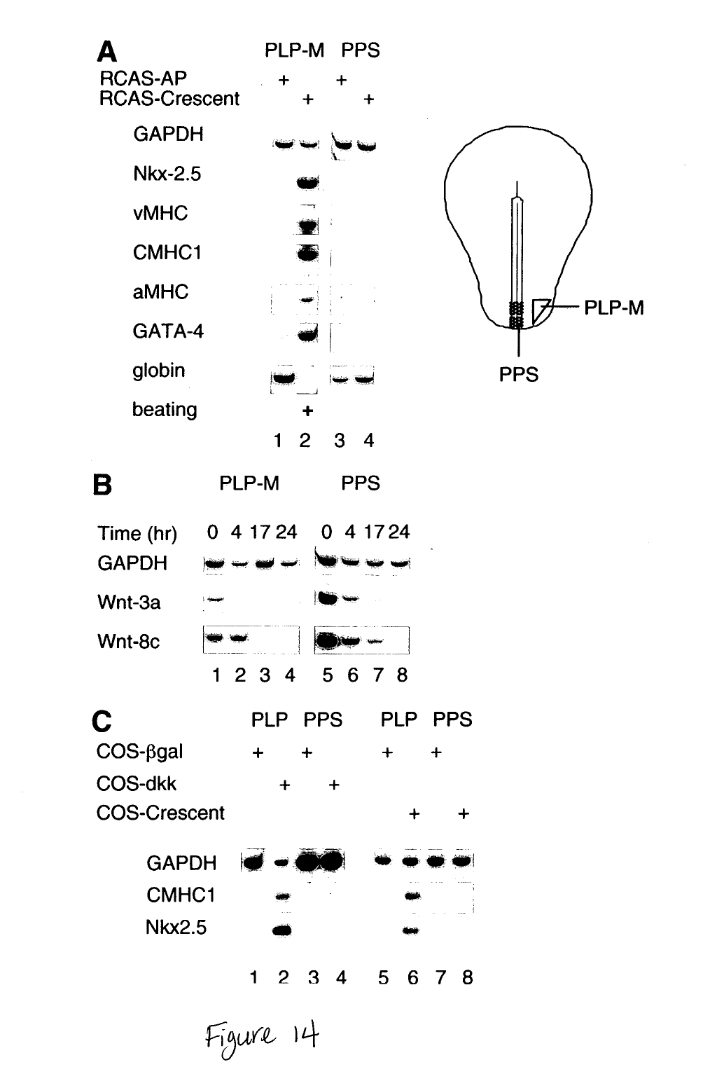

Our results with in vitro explant cultures suggest that Wnt signals from the dorsal neural tube work together with BMP-antagonists from the notochord to block ectopic cardiogenesis in anterior paraxial mesoderm. To test if such is the case in vivo, we examined whether ectopic expression of either BMP4 and/or FrzB-IgG in the anterior paraxial mesoderm could alter the fate of these cells in vivo. Pellets of 293 cells programmed to express either BMP4, FrzB-IgG, the combination of both BMP-4 and FrzB-IgG, or control IgG were implanted into the presumptive anterior paraxial mesoderm on the left side of a stage 7 chick embryo (schematically depicted in FIG. 4A) Such manipulated embryos were evaluated for Nkx-2.5 and vMHC gene expression at stages 10-14. Consistent with prior findings (Schultheiss et al. 1997; Schlange et al. 2000) and similar to our in vitro results (see above), ectopic expression of BMP-4 but not Frzb-IgG in the anterior paraxial mesoderm induced ectopic Nkx-2.5 expression in the head region (data not shown). While implantation of cells expressing only BMP-4 or Frzb-IgG into the presumptive anterior paraxial mesoderm failed to affect subsequent vMHC expression (FIG. 4B; data not shown), implantation of cells expressing the combination of BMP-4 plus Frzb-IgG resulted in increased vMHC staining in an enlarged heart (FIG. 4C). In addition, heart looping was reversed in >50% of embryos containing the BMP-4 plus Frzb-IgG cell pellets (n=25; (FIG. 4C, G). In contrast, heart looping was not affected in embryos containing either control or Frzb-IgG cell pellets (FIGS. 4B, G; data not shown), and implantation of cell pellets expressing only BMP-4 led to reverse heart looping in only 20% of such manipulated embryos (n=25; FIG. 4G). These results suggest that administration of BMP-4 plus a Wnt antagonist to the anterior paraxial mesoderm led to an increase in the pool of cardiac myocyte precursors with a corresponding enlargement of the heart. Furthermore, whereas prior studies have shown that differential BMP signaling on the left and right sides of gastrula stage chick embyros can modulate heart looping (Rodriguez Esteban et al. 1999; Yokouchi et al. 1999; Zhu et al. 1999), our findings suggest that Wnt signaling may also play a role in this process.

We speculated that the combination of BMP plus anti-Wnt signals in the presumptive anterior paraxial mesoderm may have induced the formation of an enlarged heart by converting presumptive paraxial mesodermal cells into cardiac precursors. Because cardiac precursors are known to migrate to the ventral midline under the control of sphingosine-1-phosphate (Kupperman et al. 2000), we reasoned that respecification of presumptive paraxial mesodermal cells into heart cells would result in the migration of such newly recruited cardiac myocyte precursors into the forming heart. To evaluate if implantation of cell pellets expressing BMP-4 plus Frzb-IgG caused presumptive paraxial mesoderm cells to migrate into the heart, we followed the movement of DiI-labeled head mesenchyme cells following implantation of the cell pellets (FIGS. 4D-G). After implantation of transfected 293 cell pellets into the APMEE of stage 7 chick embryos, DiI was injected between the cell pellet and the midline, as illustrated in FIG. 4A. Whereas in all embryos receiving the control IgG cell pellets the DiI-labeled cells remained at or close to the injection site (FIGS. 4D, G; n=23), in ˜80% of the embryos implanted with cell pellets expressing both BMP-4 plus Frzb-IgG (n=22), the DiI-labeled cells had migrated from the head region toward, and in some cases into, the heart (FIG. 4F, G). In contrast, only 18% of embryos containing cell pellets expressing BMP-4 plus control IgG (n=22) displayed DiI-labeled cells in the heart region (FIG. 4E, G). Thus, administration of Frzb-IgG to the presumptive head mesenchyme markedly enhanced the ability of BMP signals to induce these cells to migrate toward and into the forming heart.

To evaluate if any DiI-labeled cells in embryos that had received both BMP-4 and Frzb-IgG cell pellets expressed the cardiac marker, vMHC, we photooxidized the DiI and evaluated vMHC expression by in situ hybridization. Indeed, we observed that administration of BMP-4 plus Frzb-IgG to the presumptive head mesenchyme caused these cells to, in some cases, migrate into regions of the heart that expressed vMHC (FIG. 4H, I). Expression of vMHC was never observed in DiI-labeled cells in embryos that had received control IgG cell pellets (data not shown). These findings indicate that the combination of BMP and anti-Wnt signals can induce presumptive anterior paraxial mesodermal cells to both migrate into the heart and express a cardiac myocyte differentiation marker in vivo and are consistent with our in vitro results, suggesting that Wnt signals from the neural tube and anti-BMP signals from the notochord block cardiogenesis in this tissue (see FIG. 5).

Our findings indicate that signals from the anterior neural tube in stage 9 chick embryos prevent ectopic cardiogenesis from occurring in anterior paraxial mesendoderm and that these signals can be mimicked by either Wnt-1 or Wnt-3a expressed in the neural tube. Whereas APMEE explants cultured alone efficiently activated the cardiac program, the cardiac program was blocked in APMEE explants when cultured in the presence of the axial tissues unless both a Wnt antagonist and BMP were added. Thus, in addition to Wnt signals from the neural tube, BMP antagonists secreted by the axial tissues, such as noggin and chordin, work in combination to repress cardiogenesis in the anterior paraxial mesoderm. We suspect that repression of cardiogenesis by signals reported to come from the neural plate or neural folds in amphibians (Jacobson 1960, 1961; Raffin et al. 2000) and the notochord in zebrafish (Goldstein and Fishman 1998) may similarly reflect the expression of either Wnts or anti-BMPs in these tissues.

In Drosophila, the BMP family member, dpp (Frasch 1995), and the Wnt family member, wingless (Wu et al. 1995), are required for the maintained expression of the NK homeobox gene tinman and for subsequent cardiogenesis. Although in vertebrates BMP signals play a positive role in promoting the expression of the NK homeobox gene, Nkx-2.5, and subsequent heart formation (Schultheiss et al. 1997; Schlange et al. 2000), our findings indicate that Wnt signals paradoxically repress heart formation in vertebrates. A simple explanation for this discrepancy is that heart precursors in flies are generated in the dorsal mesoderm, adjacent to the wingless expression domain in the ectoderm, while in vertebrates, cardiac progenitors arise in regions of low or absent Wnt signaling (Marvin et al. 2001; Schneider and Mercola 2001). This redeployment of signals to control heart development may reflect a fundamental difference between the metameric origin of the Drosophila heart precursors versus the induction of a heart field in the anterior domain of vertebrate embryos. On the basis of our prior findings, we propose that newly invaginated mesodermal cells in the anterior region of the chick embryo are uniformly exposed to a cardiac-inducing signal from the anterior endoderm (Schultheiss et al. 1995). In gastrula stage embryos, Wnt antagonists promote heart formation in the anterior lateral mesoderm, while Wnt signaling in the posterior of the embryo blocks ectopic heart formation in posterior lateral mesoderm (Marvin et al. 2001; Schneider and Mercola 2001). In neurula stage embryos, progression of cells within the cardiac field to the cardiac fate is subsequently repressed in the dorsomedial region of this field by both Wnt signals and anti-BMPs secreted by the axial tissues. Conversely, cardiogenesis is promoted in the ventrolateral region of the heart field by the presence of BMPs and the absence of Wnt signals (FIG. 5).

Materials & Methods

Cell Culture

Explant culture conditions and retroviral reagents are described in Marvin et al. (2001). The CRD region (amino acids 24-178) of chick Frzb was cloned in-frame into the BamH1 site of the pRK5-IgG expression vector (human IgG heavy chain provided by J. Nathans, Johns Hopkins, Baltimore, Md.). Expression vehicles encoding either Frzb-IgG or control IgG were transfected into HEK-293 cells. Medium conditioned for 5 d was harvested and diluted 1:4 into culture medium. Alternatively, cell pellets were made from HEK-293 cells that had been transfected with either a Frzb-IgG or a control IgG expression vehicle. Noggin-conditioned medium from CHO-transfected cells (provided by R. Harland, UC Berkeley, CA) was diluted as mentioned above. Human recombinant BMP-2 (or BMP-4) was generously provided by Genetics Institute and was employed at 40-60 ng/mL. Explants were maintained in culture for 48 h unless otherwise indicated.

RT-PCR

RT-PCR was performed as described in Marvin et al. (2001).

New Culture and DiI Experiments

Stage 6-7 chick embryos were explanted ventral side up in New culture. DiI was injected into the head mesenchyme region (FIG. 4A) as described (Psychoyos and Stem 1996). Combined DiI labeling followed by in situ hybridization was performed by photoconverting the fluorescence signal before initiating the in situ hybridization protocol as described in Nieto et al. (1995).

References: Bhanot, P., Brink, M., Samos, C. H., Hsieh, J. C., Wang, Y., Macke, J. P., Andrew, D., Nathans, J., and Nusse, R. 1996. A new member of the frizzled family from Drosophila functions as a Wingless receptor. Nature 382: 225-230; Christ, B. and Ordahl, C. P. 1995. Early stages of chick somite development. Anat. Embryol 191: 381-396; Climent, S., Sarasa, M., Villar, J. M., and Murillo-Ferrol, N. L. 1995. Neurogenic cells inhibit the differentiation of cardiogenic cells. Dev. Biol. 171: 130-148; Croissant, J. D., Carpenter, S., and Bader, D. 2000. Identification and genomic cloning of CMHC1: A unique myosin heavy chain expressed exclusively in the developing chicken heart. J. Biol. Chem. 275: 1944-1951; Frasch, M. 1995. Induction of visceral and cardiac mesoderm by ectodermal Dpp in the early Drosophila embryo. Nature 374: 464-467; Goldstein, A. M. and Fishman, M. C. 1998. Notochord regulates cardiac lineage in zebrafish embryos. Dev. Biol 201: 247-252; Jacobson, A. G. 1960. Influences of ectoderm and endoderm on heart differentiation in the newt. Dev. Biol 2: 138-154; - - - . 1961. Heart determination in the newt. J. Exp. Zool. 146: 139-152; Kupperman, E., An, S., Osborne, N., Waldron, S., and Stainier, D. Y. 2000. A sphingosine-1-phosphate receptor regulates cell migration during vertebrate heart development. Nature 406: 192-195; Leyns, L., Bouwmeester, T., Kim, S. H., Piccolo, S., and De Robertis, F. M. 1997. Frzb-1 is a secreted antagonist of Wnt signaling expressed in the Spemann organizer. Cell 88: 747-756; Liem, K. F., Jr., Tremml, G., Roelink, H., and Jessell, T. M. 1995. Dorsal differentiation of neural plate cells induced by BMP-mediated signals from epidermal ectoderm. Cell 82: 969-979; Marvin, M., Rocco, G. D., Gardiner, A., Bush, S., and Lassar, A. 2001. Inhibition of Wnt activity induces heart formation from posterior mesoderm. Genes & Dev. 15: 316-327; Nieto, M. A., Sechrist, J., Wilkinson, D. G., and Bronner-Fraser, M. 1995. Relationship between spatially restricted Krox-20 gene expression in branchial neural crest and segmentation in the chick embryo hindbrain. EMBO J 14: 1697-1710; Psychoyos, D. and Stern, C. D. 1996. Restoration of the organizer after radical ablation of Hensen's node and the anterior primitive streak in the chick embryo. Development 122: 3263-3273; Raffin, M., Leong, L. M., Rones, M. S., Sparrow, D., Mohun, T., and Mercola, M. 2000. Subdivision of the cardiac Nkx2.5 expression domain into myogenic and nomnyogenic compartments. Dev. Biol. 218: 326-340; Rattner, A., Hsieh, J. C., Smallwood, P. M., Gilbert, D. J., Copeland, N. G., Jenkins, N. A., and Nathans, J. 1997. A family of secreted proteins contains homology to the cysteine-rich ligand-binding domain of frizzled receptors. Proc. Natl. Acad. Sci. 94: 2859-2863; Rodriguez Esteban, C., Capdevila, J., Economides, A. N., Pascual, J., Ortiz, A., and Izpisua Belmonte, J. C. 1999. The novel Cer-like protein Caronte mediates the establishment of embryonic left-right asymmetry. Nature 401: 243-251; Schlange, T., Andree, B., Arnold, H., and Brand, T. 2000. BMP2 is required for early heart development during a distinct time period. Mech. Dev. 91: 259-270; Schneider, V. and Mercola, M. 2001. Wnt antagonism initiates cardigenesis in Xenopus laevis. Genes & Dev. 15: 304-315; Schultheiss, T. M., Xydas, S., and Lassar, B. 1995. Induction of avian cardiac myogenesis by anterior endoderm. Development 121: 4203-4214; Schultheiss, T., Burch, J., and Lassar, A. 1997. A role for bone morphogenetic proteins in the induction of cardiac myogenesis. Genes & Dev. 11: 451-462; Wang, S., Krinks, M., Lin, K., Luyten, F. P., and Moos, M., Jr. 1997. Frzb, a secreted protein expressed in the Spemann organizer, binds and inhibits Wnt-8. Cell 88: 757-766; Wu, X., Golden, K., and Bodmer, R. 1995. Heart development in Drosophila requires the segment polarity gene wingless. Dev. Biol. 169: 619-628; Yokouchi, Y., Vogan, K. J., Pearse, R. V., II, and Tabin, C. J. 1999. Antagonistic signaling by Caronte, a novel Cerberus-related gene, establishes left-right asymmetric gene expression. Cell 98: 573-583; and Zhu, L., Marvin, M. J., Gardiner, A., Lassar, A. B., Mercola, M., Stern, C. D., and Levin, M. 1999. Cerberus regulates left-right asymmetry of the embryonic head and heart. Curr. Biol. 9: 931-938.

### Example 2

### Wnt Antagonism Initiates Cardiogenesis in Xenopus laevis

The heart in all vertebrates arises from paired regions of cardiogenic mesoderm located in dorsoanterior mesoderm. In Xenopus, this tissue lies within a portion of the equatorial region of the embryo (the marginal zone) located between 30° and 45° to either side of the dorsal midline flanking the Spemann organizer. Heart induction is largely complete by early gastrulation (Sater and Jacobson 1989, 1990; Nascone and Mercola 1995).

The Spemann organizer and the dorsoanterior endoderm that underlies the precardiac mesoderm are both necessary for induction and together are sufficient to induce beating heart tissue in noncardiogenic ventral marginal zone mesoderm (Nascone and Mercola 1995). Heart induction in Xenopus resembles the same process in avians, in which the cardiogenic mesoderm, located on either side of the anterior primitive streak, is induced by interactions with underlying definitive endoderm (Antin et al. 1994; Sugi and Lough 1994; Schultheiss et al. 1995).

Although several proteins have been implicated in the induction of cardiogenic mesoderm, their specific roles in this process are not entirely clear and additional factors are likely to be involved. Members of the bone morphogenetic protein (BMP) family are expressed adjacent to the heart-forming region in avians, and ectopic expression of the BMP antagonist noggin in chick precardiac mesoderm inhibits cardiogenesis (Schultheiss et al. 1997; Schlange et al. 2000). Conversely, application of BMP2 or BMP4 to chick anterior mesoderm located medial to the heart forming region induces ectopic cardiogenesis (Schultheiss et al. 1997; Andree et al. 1998). However, these BMPs cannot mimic the ability of endoderm to induce cardiogenesis in more posterior mesoderm, indicating the involvement of additional factors (Schultheiss et al. 1997). Two lines of experiments using Xenopus embryos also indicate that factors other than BMPs are required for initiation of cardiogenesis. First, inhibition of endogenous BMP signaling with a dominant negative type I receptor blocked maintenance but not initial expression of Nkx2.5, a homolog of the Drosophila tinman gene and an early marker of heart field specification (Shi et al. 2000). Second, mRNAs encoding BMP isoforms are not expressed by either of the tissues known to have heart-inducing activity, the dorsoanterior endoderm or the Spemann organizer (Isaacs et al. 1992, 1995; Tannahill et al. 1992; Suzuki et al. 1993; Song and Slack 1994; Clement et al. 1995; Yamagishi et al. 1995; Jones et al. 1996). In avians, fibroblast growth factor (FGF) family members have been proposed to work in conjunction with BMPs, but in Xenopus, their mRNAs are also not expressed in heart-inducing tissues, again suggesting the participation of additional factors in cardiogenesis.

Studies have also indicated that an activin-like activity might be involved in heart induction. Treatment of avian posterior epiblast tissue with activin-induced cardiac myogenesis (stage XI-XIV, staging according to Eyal-Giladi and Kochav 1976; Yatskievych et al. 1997; Ladd et al. 1998). However, the inability of this protein to induce heart muscle cells in streak stage mesodermal explants (the period when heart induction normally occurs) indicate that the role of activin in this process might be indirect, possibly by promoting the formation of precardiac mesoderm competent to respond to heart-inducing signals. Similarly, induction of cardiogenesis in Xenopus animal cap tissue by ectopic activin expression correlates with formation of both dorsal mesoderm and endoderm (Logan and Mohun 1993; Henry et al. 1996), raising the possibility that heart induction occurred because of interactions between these tissues.

Finally, several experiments have implicated Cerberus, a member of the DAN family of secreted proteins that inhibit signaling by BMP, Wnt, and Nodal-related proteins, in cardiogenesis (Bouwmeester et al. 1996; Hsu et al. 1998; Pearce et al. 1999; Piccolo et al. 1999; Belo et al. 2000). Cerberus homologs are expressed in heart-inducing tissues in mouse (Belo et al. 1997; Biben et al. 1998; Shawlot et al. 1998), chick (Esteban et al. 1999; Yokouchi et al. 1999; Zhu et al. 1999), and Xenopus (Bouwmeester et al. 1996; Schneider and Mercola 1999) and can induce expression of Nkx2.5 in Xenopus animal cap tissue (Bouwmeester et al. 1996; Belo et al. 1997; Biben et al. 1998). However, as Cerberus does not induce expression of markers of terminal cardiac differentiation (Biben et al. 1998; V. Schneider and M. Mercola, unpubl.) and hearts develop in mice lacking the murine homolog Cerberus-like (Simpson et al. 1999; Belo et al. 2000), the cardiogenic function of Cerberus proteins, if any, remains elusive. Taken together, these data indicate that additional factors are necessary to initiate cardiogenesis in both vertebrate embryos.

The requirement for the Spemann organizer in heart induction led us to ask whether organizer-derived factors have heart-inducing activity. Secreted factors produced by the Spemann organizer in Xenopus have been studied intensely and shown to be important for pattern formation both before and during gastrulation (for review, see Harland and Gerhart 1997). Dorsalizing activity of the organizer is mediated by Nodal-like signaling as well as by specific antagonists of BMP (Chordin and Noggin) and Wnt signaling (Frzb, Dkk-1, and Crescent; Sasai et al. 1994; Jones et al. 1995; Zimmerman et al. 1996; Leyns et al. 1997; Wang et al. 1997a; Glinka et al. 1998; Pera and De Robertis 2000). Embryological studies of these proteins have revealed potent dorsoanteriorizing effects on the mesoderm and ectoderm. Importantly, antagonism of Wnt and BMP activities are not entirely redundant but appear complementary. For instance, Glinka et al. (1997) provided evidence that inhibition of BMP signaling alone results in tail organizing activity, whereas inhibition of both BMP and Wnt pathways promotes the generation of head structures anterior to the midhindbrain. Thus, both the expression of BMPs and Wnts and their inhibition are important aspects of the generation of early embryonic pattern. Moreover, at least one Wnt (Wnt11) has been implicated in early chick cardiogenesis (Eisenberg and Eisenberg 1999).

Here we show that expression of the Wnt antagonists Dkk-1 and Crescent is sufficient to induce heart formation in noncardiogenic ventral marginal zone mesoderm. This activity is not shared by other antagonists of Wnt signaling, nor the BMP antagonists Noggin and Chordin, indicating that inhibition of specific Wnts may be required. Analysis of Wnt proteins expressed at the onset of gastrulation indicated that only Wnt3A and Wnt8, but not Wnt5A and Wnt11, were capable of inhibiting endogenous heart induction. The data indicate a model in which diffusion of Dkk-1 and Crescent from the Spemann organizer region initiates cardiogenesis in the immediately adjacent mesoderm by creating a zone of reduced Wnt3A and Wnt8 activity. Dkk-1 and Crescent, but not Frzb, can induce heart-specific gene expression in noncardiogenic mesoderm

Our previous studies showed that beating hearts having lumens lined by endothelial cells can be induced in explants of noncardiogenic ventral marginal zone (VMZ) mesoderm by exposure to both the Spemann organizer and dorsoanterior endoderm (Nascone and Mercola 1995). In a modification of this assay (FIG. 6A), we targeted mRNAs encoding Wnt and BMP antagonists to VMZ tissue by microinjection into the equatorial region of both ventral blastomeres of four-cell stage embryos. VMZ explants were isolated at stage 10, cultured, and assayed at stage 30 by RT-PCR for cardiac-specific gene expression.

dkk-1 encodes a secreted protein capable of antagonizing Wnt signaling that is normally expressed in the Spemann organizer region of stage 10 embryos (Glinka et al. 1998). We find that ectopic expression of dkk-1 in VMZ explants at doses of 450 pg or greater induces abundant expression of Nkx2.5 and Tbx5, two homeobox genes that mark the early heart field (FIG. 6B; Tonissen et al. 1994; Newman and Krieg 1998; Horb and Thomsen 1999). In addition, the same doses of dkk-1 also promote the strong expression of TnIc and MHCα, which encode cardiomyocyte-specific contractile proteins (FIG. 6B; Logan and Mohun 1993; Drysdale et al. 1994). In situ hybridization demonstrated that TnIc transcripts were highly localized in the VMZ explants (FIG. 6C).

crescent encodes a Wnt antagonist containing a frizzled-like cysteine-rich domain that is also expressed in the Spemann organizer region in a pattern overlapping that of dkk-1 (Pera and De Robertis 2000). We find that crescent, like dkk-1, is a potent inducer of both early and late heart-specific gene expression in VMZ tissue (FIG. 6B). Robust expression of cardiac-specific genes was induced following injection of 900 pg of chick crescent mRNA, slightly more than required with dkk-1. However, doses of crescent as low as 180 pg induced expression of muscle actin, which primarily marks skeletal muscle (but is also expressed in cardiac muscle). As seen with dkk-1, TnIc expression induced by crescent was highly localized (FIG. 6D).

The reason for the difference in doses of dkk-1 and crescent mRNA required to induce muscle actin and the cardiac-specific markers was explored further by evaluating their relative ability to block Siamois induction by Wnt8 (FIG. 6F, G). Injection of dkk-1 mRNA yielded more potent Wnt8 antagonism than did crescent mRNA (FIG. 6G), indicating that differential antagonism of Wnt8 (or other Wnt proteins) might underlie the different activities of these two proteins. The difference in the activities of these proteins, however, could also reflect variations in the translational efficiency of their mRNAs. Nonetheless, our data show that Dkk-1 and Crescent are both potent inducers of cardiac-specific gene expression in the VMZ.

Dkk-1 and Crescent also induced Nk2.10, which encodes a transcription factor with homology to Nkx2.5 (FIG. 6B). Whereas transcripts for Nkx2.5 are present in both cardiac mesoderm and the underlying pharyngeal endoderm of stage 30 embryos, Nkx2.10 mRNA marks only the endodermal portion of the Nkx2.5 domain at this stage (Newman and Krieg 1998; Newman et al. 2000). The observed induction of Nkx2.10 therefore indicates that both Dkk-1 and Crescent induced pharyngeal endoderm along with cardiac mesoderm in VMZ tissue. This could occur if Dkk-1 and Crescent dorsoanteriorized the deep endoderm contained in our VMZ explants that would normally contribute to posterior regions of the gut.

Of the three Wnt antagonists known to be expressed in the Spemann organizer, only Frzb was incapable of inducing expression of genes encoding heart muscle-specific proteins in VMZ tissue (FIG. 6B, E). Despite this, microinjection of frzb mRNA efficiently induced muscle actin in VMZ tissue (FIG. 6B), antagonized Wnt8 induction of Siamois in animal caps (FIG. 6G), and produced shortened body axes when injected ventrally into embryos at the four-cell stage (data not shown), demonstrating that a lack of protein production was not likely to be responsible for this result. frzb weakly induced expression of Nkx2.5 and Tbx5 detectable by RT-PCR (FIG. 6B) but not by in situ hybridization (FIG. 6E). Tbx5, however, is also expressed in the eye at this stage (Horb and Thomsen 1999), and we observed induction of the pharyngeal endoderm marker Nkx2.10, which overlaps Nkx2.5 expression (FIG. 6B). Thus, we cannot distinguish whether ectopic Frzb in VMZ explants weakly induced early but not late stages of cardiogenesis and/or pharyngeal endoderm or, instead, activated expression of the NK2 family of genes in the absence of either heart or pharyngeal induction. The lack of heart-marker induction by Frzb may reflect a difference in the affinities of Wnt antagonists for various Wnt family members and raises the possibility, addressed below, that specific Wnts negatively regulate heart induction.

Expression of dkk-1 and Crescent in VMZ Explants Results in the Formation of Beating Hearts

To determine whether dkk-1 and crescent could promote later stages of cardiogenesis, we cultured VMZ explants injected with these mRNAs to stage 41, when beating hearts were apparent in control embryos. Remarkably, as heart induction is known to require both endodermal and organizer derived signals, we found that the injection of a single mRNA was sufficient to promote terminal cardiac differentiation. Rhythmic beating was observed on average in 73.2% of explants (n=44) injected with dkk-1 and in 23.2% (n=90) with crescent (FIG. 7A). Uninjected VMZ control explants, in contrast, were never observed to beat (n=66). frzb, which did not induce heart-specific gene expression in VMZ explants, was also unable to induce beating (n=35). Strikingly, the dkk-1- and crescent-injected VMZ explants retained their ventral appearance, except for features of cardiogenesis. Explants generally formed round vesicles encapsulating beating heart tissue, with few other identifiable structures (FIG. 7). Superficially, this appearance resembled uninjected control explants and differed greatly from either VMZ explants injected with either noggin or chordin or DMZ explants, all of which developed an elongated anteroposterior body axis (FIGS. 7, cf E, H to characteristic dorsal appearance of a DMZ explant, panel B). Expression of dkk-1 or crescent mRNAs was noted, however, to cause an increase in melanocyte formation and to induce cement glands in these VMZ explants (90.7% and 61.2%, respectively).

Histological sections through representative explants are shown in FIG. 7. Immunohistochemical staining with the polyclonal antibody CT-3, which recognizes the cardiac-specific isoform of troponin-T, revealed that both dkk-1 (FIGS. 7F, G) and crescent (FIG. 71,J) induced myocardial tubes. In all cases, the lumens of the myocardial tubes were lined by a thin layer of endothelial cells that do not stain with CT-3 (arrows in FIGS. 7D, G, J). We conclude that both dkk-1 and crescent are sufficient to induce terminal cardiogenesis and that the ectopic hearts exhibit the morphology and gene expression characteristic of hearts that develop in intact embryos or in control DMZ explants that contain normal cardiac tissue (FIGS. 7C, D).

The BMP Antagonists Noggin and Chordin do not Induce Cardiac-Specific Gene Expression in VMZ Explants

Induction of cardiogenesis by Dkk-1 and Crescent led us to ask whether such activity is shared by the BMP antagonists Noggin and Chordin, which also dorsalize mesoderm, or whether it is a specific property of particular Wnt antagonists. Noggin and chordin are of interest because, like dkk-1, crescent, and frzb, they are normally expressed in the Spemann organizer. Injection of all doses of noggin mRNA tested resulted in extensive elongation of VMZ explants and doses >50 pg caused such extreme morphogenetic movements that explants were unable to survive until stages at which heart development could be analyzed. Doses of noggin as low as 5 pg, however, were potent inducers of dorsal mesoderm in VMZ explants, as seen by the induction of muscle actin (data not shown). None of the doses of noggin injected, ranging from 5 to 50 pg, were able to induce expression of either early or late heart markers, as compared with uninjected VMZ explants (FIGS. 8A, E, E′; data not shown).

Injection of chordin mRNA caused VMZ explants to elongate and form embryoids having anteriorly truncated body axes (FIGS. 8A, F, F′; data not shown), and RT-PCR analysis confirmed the induction of muscle actin (FIG. 8A). In contrast to noggin, chordin was also observed to induce low-level expression of Nkx2.5 and Tbx5 (FIG. 8A). As with frzb, Nkx2.5 expression after chordin injection was not detectable by in situ hybridization (FIG. 8F), indicating only weak induction. Moreover, no dose tested (ranging from 180 pg to 1.5 ng) could induce contractile protein mRNAs (FIGS. 8A, F′; data not shown). The induction of the pharyngeal marker Nkx2.10 indicates that Chordin, well known to dorsalize ectoderm (Lamb et al. 1993), also dorsoanteriorized the endoderm present in the VMZ explants. Thus, we cannot distinguish whether Chordin, like Frzb, weakly induced early stages of cardiogenesis or activated NK2 family members in the absence of heart (or pharyngeal endoderm) induction. Despite the uncertain role of Chordin, it is clear that the induction of heart-specific mRNAs in VMZ explants is a specific property of Wnt antagonism rather than a general feature of dorsalization as mediated by BMP antagonism.

Wnt Antagonists other than Dkk-1 and Crescent are Unable to Induce Heart-Specific mRNA Expression in VMZ Explants

To characterize the range of Wnt antagonists capable of heart induction, we examined representatives of three different classes of inhibitors: dominant negative Xenopus Wnt8 (Hoppler et al. 1996), WIF-1 (a WIF domain antagonist; Hsieh et al. 1999), and FrzA and Szl (frizzled domain antagonists; Salic et al. 1997; Xu et al. 1998). Injection of as much as 1.5 ng of dnXwnt8, which is known to inhibit Wnt1, Wnt3A, and Wnt8 (Hoppler et al. 1996), was unable to induce expression of muscle actin above levels found in control VMZ explants (FIG. 8B). In addition, only weak induction of Nkx2.5 and 2.10 was observed in dnXwnt8-injected VMZ explants. Notably, dnXwnt8 did not induce expression of the heart-specific mRNAs TnIc and MHCα in our experiments (FIG. 8B). The inability to induce heart-specific mRNAs was apparently not due to lack of protein production, as doses of dnXwnt8 as low as 45 pg were effective at inhibiting Siamois induction in animal caps by Xwnt8 (data not shown). Similarly, WIF-1, frzA, and szl only weakly induced XNkx2.5 and 2.10 at the highest doses tested, and none induced the heart-specific contractile protein genes TnIc and MHCα (FIG. 8B). Of these Wnt antagonists, only WIF-1 induced expression of Nkx2.5 at levels detectable by in situ hybridization (FIGS. 8G-J), and none induced detectable levels of TnIc transcripts (FIGS. 8G′-J′). Sibling embryos injected with each of these mRNAs, but not dissected for VMZ explants, developed malformations characteristic of each inhibitor, indicating that the injected mRNAs yielded functional protein (Wu et al. 1995; Salic et al. 1997; Hsieh et al. 1999; data not shown). Thus, of the Wnt antagonists examined, only Dkk-1 and Crescent induced ectopic cardiogenesis in VMZ tissue. Previous studies have demonstrated that the various antagonists have differing abilities to block signaling from different Wnt proteins (Wang et al. 1997b; Xu et al. 1998; Dennis et al. 1999; Krupnik et al. 1999). We conclude that Dkk-1 and Crescent, which are present in the gastrula stage organizer region, induce cardiogenesis in VMZ tissue by the selective inhibition of one or more endogenous Wnt proteins.

GSK3β, an Inhibitor of β-Catenin-Mediated Wnt Signaling, Induces Expression of Heart-Specific Genes in VMZ Explants

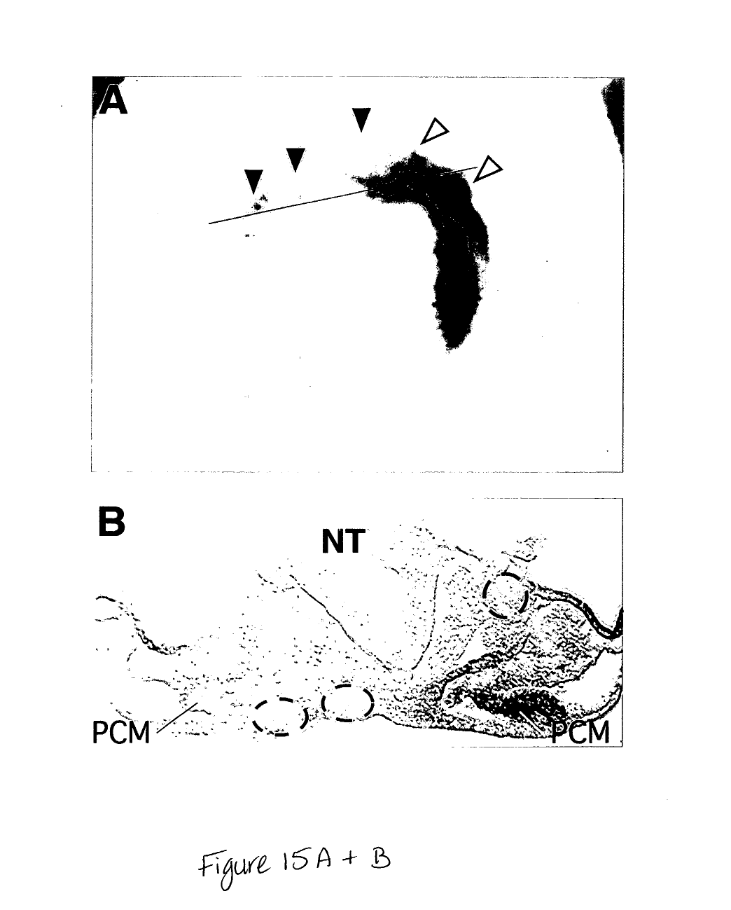

Wnt signaling is transduced by at least two different pathways, one that depends on transcription mediated by β-catenin and a second that involves the stimulation of protein kinase C (for review, see Moon et al. 1997; Sheldahl et al. 1999; Kuhl et al. 2000). To determine if β-catenin signaling must be inhibited for cardiogenesis to proceed, we tested whether the serine/threonine kinase GSK3p would induce heart-specific gene expression in VMZ explants. Phosphorylation by GSK3 targets β-catenin for ubiquitination and ultimate degradation (Aberle et al. 1997). As before, mRNA encoding GSK3β was injected ventrally at the four-cell stage and VMZ explants were analyzed for cardiac specific gene expression. GSK3p did not induce appreciable expression of muscle actin, indicating relatively weak dorsalizing ability in VMZ tissue. Like dkk-1 and crescent, however, GSK3β yielded robust induction of each of the cardiac-specific genes, including TnIc and MHCα (FIG. 9). This finding indicates that inhibition of β-catenin is sufficient to induce cardiogenesis.

Overexpression of Wnt3A or Wnt8 Blocks Cardiogenesis in DMZ Explants

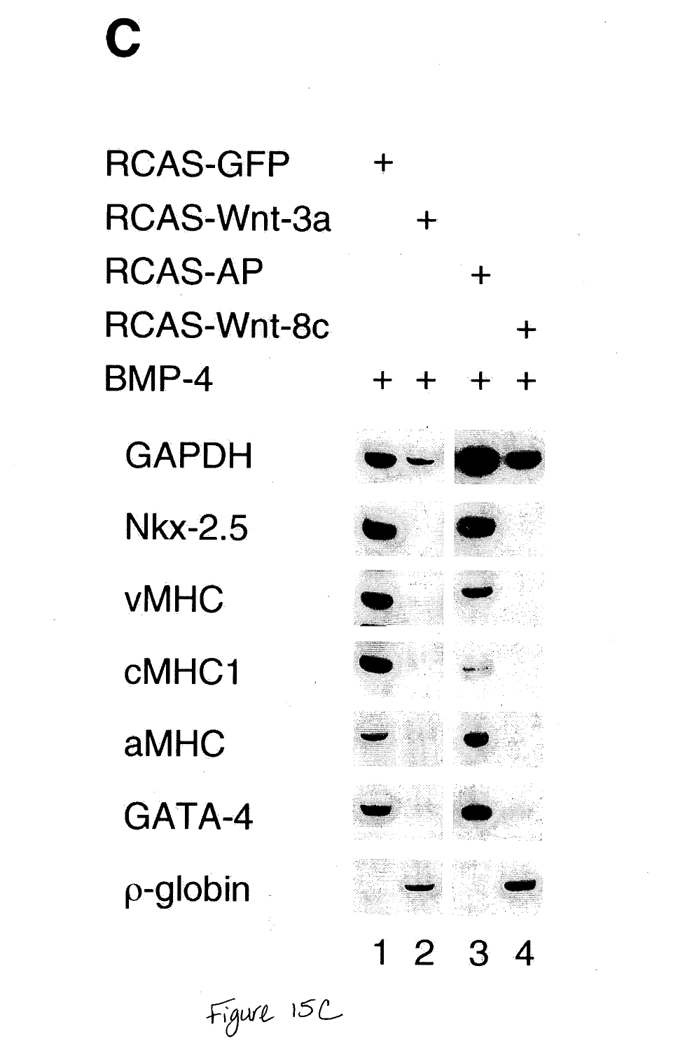

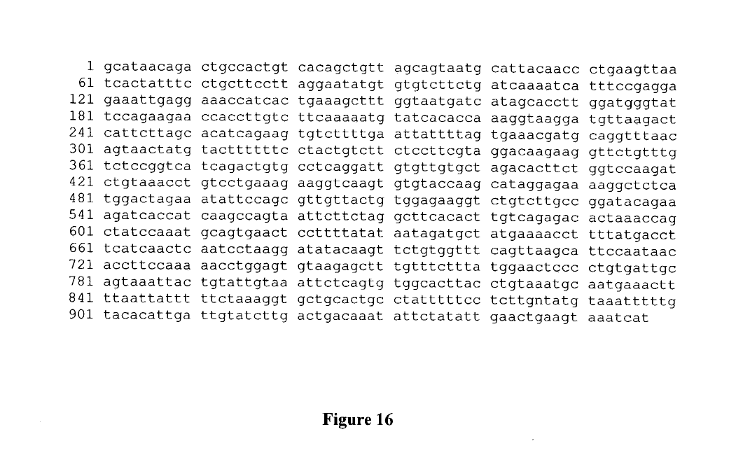

The preceding experiments demonstrated that inhibition of Wnt signaling is sufficient to promote cardiogenesis in noncardiogenic ventral tissue. If the normal function of Wnt antagonism in vivo is to induce cardiogenic mesoderm, then overexpression of Wnt proteins should block cardiogenesis in dorsal mesoderm. Four Wnt genes are known to be expressed during gastrulation: Wnt3A, Wnt5A, Wnt8, and Wnt11. Expression of Wnt8 is normally excluded from the organizer region, whereas Wnt 3A and Wnt11 are expressed dorsally and Wnt5A is found diffusely throughout the ectoderm (Christian and Moon 1993; Ku and Melton 1993; Moon et al. 1993; Du et al. 1995; McGrew et al. 1997). We injected Wnt cDNAs into the two dorsal blastomeres of a four-cell embryo and dissected DMZ explants encompassing the organizer and heart primordia at stage 10 (FIG. 10A). Plasmid injections were performed to avoid perturbation of Nieuwkoop center activity that can occur on expression of certain Wnts before the midblastula transition (Smith and Harland 1991; Sokol et al. 1991). Explants were cultured to either stage 23 or stage 30, at which time they were examined for the expression of Nkx2.5 or TnIc. Explants were analyzed individually by in situ hybridization, rather than as pools by RT-PCR, as a decrease in the heart-marker expression of a single explant would likely escape detection if it were pooled with other samples exhibiting normal levels of expression.

FIG. 10B shows that only Wnt8 and Wnt3A were potent inhibitors of endogenous cardiac gene expression. The incidence of explants expressing Nkx2.5 decreased to 45.6% (n=62) and 19.9% (n=50) on overexpression of Wnt3A and Wnt8, respectively, compared with 98.3% (n=65) seen in uninjected controls. Injection of these same Wnts also caused the incidence of TnIc expression decline substantially, to 24.2% (n=62) and 41.1% (n=254), respectively, from 94.5% (n=147) in controls. (FIG. 10B). Interestingly, TnIc expression was either absent (FIGS. 10C, D) or greatly reduced in area (FIGS. 10C′, D′). Whereas dorsal overexpression of Wnt8 or Wnt3A prevented specification of the heart field, overexpression of Wnt5A and Wnt11 did not appreciably affect the incidence of either Nkx2.5 (97.5%, n=35 and 94.1%, n=35, respectively) or TnIc expression (85.9%, n=58 and 83.1%, n=51, respectively; FIG. 10B). Moreover, the expression domains of both heart markers appeared normal (FIGS. 10, cf. E, F to control explant in G). Taken together, our data indicate a model in which at least Wnt3A and Wnt8 activity must be inhibited to specify the heart field in dorsal mesoderm adjacent to the Spemann organizer.

The principal conclusion from our experiments is that Wnt signaling through β-catenin prevents heart induction and that this inhibition is overcome on the dorsal side of the embryo via the action of specific Wnt antagonists produced by the Spemann organizer. Ectopic expression of either dkk-1 or crescent induced both early and late cardiac genetic markers in explants of noncardiogenic VMZ tissue. Remarkably, injection of a single factor induced explants to form rhythmically beating myocardial tubes that morphologically resembled normal hearts. Given the differential ligand specificity of the various Wnt antagonists, the inability of other such proteins to induce heart-specific gene expression indicated that inhibition of particular Wnts is responsible. Accordingly, overexpression of Wnt3A and Wnt8, but not other Wnts thought to be present in the gastrula-stage embryo, inhibited endogenous cardiogenesis. These results are the first demonstration of factors that initiate cardiogenesis in Xenopus.

Materials & Methods

Embryo and Explant Culture

Embryos were fertilized in vitro, dejellied in 2% cysteine-HCl (pH 7.8), and maintained in 0.1×MMR. Explant dissections were performed in 0.75×MMR using an eyelash knife. Embryos were staged according to Nieuwkoop and Faber (1994).

Marginal zone explants were dissected at stage 10. Those explants to be examined by RT-PCR for expression of heart field marker- and heart muscle-specific genes were cultured until sibling embryos were stage 30. In situ hybridization was performed on explants cultured to the equivalent of stage 23 or stage 30. Explants to be scored for formation of beating hearts were maintained until the equivalent of stage 41.

Plasmids and mRNA for Injections

mRNA was transcribed from pSP35-chd, pSP64-ngn, pCS2-DKK1, pCS2-Crescent, pCS2-GSK3,, pCS2-WIF, pCS2-dnXwnt8, pCS2-szl, and pXT7-FrzA using the SP6 and T7 mMessage mMachine kits (Ambion). All cDNAs used encode Xenopus proteins except those for Wnt1 and crescent, which encode chick isoforms. The Xenopus form of crescent was identified while this manuscript was in preparation (Pfeffer et al. 1997; Pera and De Robertis 2000; Shibata et al. 2000) and functions identically to the chick isoform in our assays. Xenopus and chick Crescent share 88% amino acid positional identity within the cysteine-rich domain. Injections were performed in 3% Ficoll in 1×MMR. Embryos were injected equatorially into the two ventral or two dorsal blastomeres at the four-cell stage to target expression to the ventral or dorsal marginal zone. The amount of mRNA injected is given in the text. For plasmid cDNA injections, 75 pg of pCS2-Xwnt3A, pCS2-Xwnt5A, and pCSKA-Xwnt8 and 100 pg of pCS2-cWnt11 supercoiled plasmid constructs were injected.

RT-PCR

RT-PCR was performed as described in Schneider and Mercola (1999). Twenty-five cycles were performed at an annealing temperature of 55° C., unless otherwise noted. Expression of EF1α was used as a positive control for the reverse transcriptase reaction. The following additional primers were used: XNkx2.5+, GAGCTACACTTGGGTGTGTGTGGT (SEQ ID NO: 7); XNkx2.5−, GTGAAGCGACTAGGTATGTGTTCA (SEQ ID NO: 8); M. actin+, GCTGACAGAATGCAGAAG (SEQ ID NO: 9); M. actin−, TTGCTTGGAGGAGTGTGT (SEQ ID NO: 10) (22 cycles); TnIc+, CTGATGAGGAAGAGGTAACC (SEQ ID NO: 11); TnIc−, CCTCACGTTCCATTTCTGCC (SEQ ID NO: 12); MHCα+, GCCAACGCGAACCTCTCCAA GTTCCG (SEQ ID NO: 13); MHCα−, GGTCACATTTTATTTCATGCT GGTTAACAGG (SEQ ID NO: 14); Tbx5+, GGCGGACACAGAGGAGGCTTAT (SEQ ID NO: 15); Tbx5−, GTGGCTGGTGAATCTGGGTGAAC (SEQ ID NO: 16) (27 cycles); XNkx2.10+, GCCCCGCTACCTCTACCCCCTTCT (SEQ ID NO: 17); and XNkx2.10−, CCCCTCTCACTGTGCCCCCAAAAT (SEQ ID NO: 18) (59° C., 28 cycles).

In Situ Hybridization

In situ hybridization was performed according to the protocol of Harland (1991). Digoxygenin-labeled probes were transcribed from the following linearized plasmids: pGEM-XNkx2.5 (XbaI, T7 polymerase) and pBS-TnIc (NotI, T7).

Immunohistochemistry

Embryos and explants were fixed in MEMFA and stored in 100% MeOH (Harland 1991). Immunohistochemistry was performed essentially as described (Hemmati-Brivanlou and Harland 1989). CT-3, which recognizes the cardiac isoform of troponin T, was used as the primary antibody (Developmental Studies Hybridoma Bank). Rhodamine-conjugated secondary antibodies were used to visualize primary antibody labeling of proteins. Following incubation with secondary antibody, samples were rinsed in 1×PBS, postfixed in MEMFA, dehydrated through an ethanol series, and embedded in paraffin (Oxford Laboratories).

Embedded explants were sectioned, deparaffinized with xylenes, rehydrated, and stained with DAPI before visualization by epifluorescence microscopy on a Zeiss Axiophot microscope.

References: Aberle, H., Bauer, A., Stappert, J., Kispert, A., and Kemler, R. 1997. β-catenin is a target for the ubiquitin-proteasome pathway. EMBO J 13: 3797-3804; Andree, B., Duprez, R., Vorbusch, B., Arnold, H. H., and Brand, T. 1998. BMP-2 induces ectopic expression of cardiac lineage markers and interferes with somite formation in chicken embryos. Mech. Dev. 70: 119-131; Antin, P. B., Taylor, R. G., and Yatskievych, T. 1994. Precardiac mesoderm is specified during gastrulation in quail. Dev. Dyn. 200: 144-154; Belo, J. A., Bouwmeester, T., Leyns, L., Kertesz, N., Gallo, M., Follettie, M., and De Robertis, E. M. 1997. Cerberus-like is a secreted factor with neuralizing activity expressed in the anterior primitive endoderm of the mouse gastrula. Mech. Dev. 68: 45-57; Belo, J. A., Bachiller, D., Agius, E., Kemp, C., Borges, A. C., Marques, S., Piccolo, S., and De Robertis, E. M. 2000. Cerberus-like is a secreted BMP and nodal antagonist not essential for mouse development. Genesis 26: 265-270; Biben, C., Stanley, E., Fabri, L., Kotecha, S., Rhinn, M., Drinkwater, C., Lah, M., Wang, C.-C., Nash, A., Hilton, D. et al. 1998. Murine cerberus homologue mCer-1: A candidate anterior patterning molecule. Dev. Biol. 194: 135-151; Bouwmeester, T., Kim, S., Sasai, Y., Lu, B., and De Robertis, E. M. 1996. Cerberus is a head-inducing secreted factor expressed in the anterior endoderm of Spemann's organizer. Nature 382: 595-601; Christian, J. L. and Moon, R. T. 1993. Interactions between Xwnt-8 and Spemann organizer signaling pathways generate dorsoventral pattern in the embryonic mesoderm of Xenopus. Genes & Dev. 7: 13-28; Clement, J. H., Fettes, P., Knochel, S., Lef, J., and Knochel, W. 1995. Bone morphogenetic protein 2 in the early development of Xenopus laevis. Mech. Dev. 52: 357-370; Dennis, S., Aikawa, M., Szeto, W., d'Amore, P. A., and Papkoff, J. 1999. A secreted Frizzled related protein, FrzA, selectively associates with Wnt-1 protein and regulates Wnt-1 signaling. J. Cell Sci. 112: 3815-3820; Drysdale, T. A., Tonissen, K. F., Patterson, K. D., Crawford, M. J., and Krieg, P. A. 1994. Cardiac troponin I is a heart-specific marker in the Xenopus embryo: Expression during abnormal heart morphogenesis. Dev. Biol. 154: 432-441; Du, S. J., Purcell, S. M., Christian, J. L., McGrew, L. L., and Moon, R. T. 1995. Identification of distinct classes and functional domains of Wnts through expression of wild-type and chimeric proteins in Xenopus embryos. Mol. Cell. Biol. 15: 2625-2634; Eisenberg, C. A. and Eisenberg, L. M. 1999. WNT 1 promotes cardiac tissue formation of early mesoderm. Dev. Dyn. 216: 45-58; Esteban, C. R., Capdevila, J., Economides, A. N., Pascual, J., Ortiz, A., and Izpisua-Belmonte, J. C. 1999. The novel Cer-like protein Caronte mediates the establishment of embryonic left-right asymmetry. Nature 410: 243-251; Eyal-Giladi, H. and Kochav, S. 1976. From cleavage to primitive streak formation: A complementary normal table and a new look at the first stages of the development of the chick. I. General morphology. Dev. Biol. 49: 321-337; Glinka, A., Wu, W., Onichtchouk, D., Blumenstock, C., and Niehrs, C. 1997. Head induction by simultaneous repression of Bmp and Wnt signalling in Xenopus. Nature 389: 517-519; Glinka, A., Wu, W., Delius, H., Monaghan, A. P., Blumenstock, C., and Niehrs, C. 1998. Dickkopf-1 is a member of a new family of secreted proteins and functions in head induction. Nature 392: 357-362; Harland, R. M. 1991. In situ hybridization: An improved whole mount method for Xenopus embryos. Methods Cell Biol. 36: 685-695; Harland, R. and Gerhart, J. 1997. Formation and function of Spemann's organizer. Annu. Rev. Cell Dev. Biol. 13: 611-667; Hemmati-Brivanlou, A. and Harland, R. M. 1989. Expression of an engrailed-related protein is induced in the anterior neural ectoderm of early Xenopus embryos. Development 106: 611-617; Henry, G. L., Brivanlou, I. H., Kessler, D. S., Hemmati-Brivanlou, A., and Melton, D. A. 1996. TGF-β signals and a prepattern in Xenopus laevis endodermal development. Development 122: 1007-1015; Hoppler, S., Brown, J. D., and Moon, R. T. 1996. Expression of a dominant-negative Wnt blocks induction of MyoD in Xenopus embryos. Genes & Dev. 10: 2805-2817; Horb, M. E. and Thomsen, G. H. 1999. Tbx5 is essential for heart development. Development 126: 1739-1751; Hsieh, J.-C., Kodjabachian, L., Rebbert, M. L., Rattner, A., Smallwood, P. M., Samos, C. H., Nusse, R., Dawid, I. B., and Nathans, J. 1999. A new secreted protein that binds to Wnt proteins and inhibits their activities. Nature 398: 431-436; Hsu, D. R., Economides, A. N., Wang, X., Eimon, P. M., and Harland, R. M. 1998. The Xenopus dorsalizing factor Gremlin identifies a novel family of secreted proteins that antagonize BMP activities. Mol. Cell 1: 673-683; Isaacs, H. V., Tannahill, D., and Slack, J. M. W. 1992. Expression of a novel FGF in the Xenopus embryo: A new candidate inducing factor for mesoderm formation and anteroposterior specification. Development 114: 711-720; Isaacs, H. V., Pownall, M. E., and Slack, J. M. W. 1995. eFGF is expressed in the dorsal midline of Xenopus laevis. Int. J. Dev. Biol. 39: 575-579; Jones, C. M., Kuelin, M. R., Hogan, B. L. M., Smith, J. C., and Wright, C. V. E. 1995. Nodal-related signals induce axial mesoderm and dorsalize mesoderm during gastrulation. Development 121: 3651-3662; Jones, C. M., Dale, L., Hogan, B. L. M., Wright, C. V. E., and Smith, J. C. 1996. Bone morphogenetic protein-4 (BMP-4) acts during gastrula stages to cause ventralization of Xenopus embryos. Development 122: 1545-1554; Krupnik, V. E., Sharp, J. D., Jiang, C., Robison, K., Chickering, T. W., Amaravadi, L., Brown, D. E., Guyot, D., Mays, G., Leiby, K. et al. 1999. Functional and structural diversity of the human Dickkopf gene family. Gene 238: 303-313; Ku, M. and Melton, D. A. 1993. Xwnt-11: A maternally expressed Xenopus wnt gene. Development 119: 1161-1173; Kuhl, M., Sheldahl, L. C., Malbon, C. C., and Moon, R. T. 2000. Ca²⁺/Calmodulin-dependent protein kinase II is stimulated by Wnt and Frizzled homologs and promotes ventral cell fates in Xenopus. J. Biol. Chem. 275: 12701-12711; Ladd, A. N., Yatskievych, T. A., and Antin, P. B. 1998. Regulation of avian cardiac myogenesis by activin/TGFβ and bone morphogenetic proteins. Dev. Biol. 204: 407-419; Lamb, T. M., Knecht, A. K., Smith, W. C., Stachel, S. E., Economides, A. N., Stahl, N., Yancopolous, G. D., and Harland, R. M. 1993. Neural induction by the secreted polypeptide noggin. Science 262: 713-718; Leyns, L., Bouwmeester, T., Kim, S.-H., Piccolo, S., and De Robertis, E. M. 1997. Frzb-1 is a secreted antagonist of Wnt signaling expressed in the Spemann organizer. Cell 88: 747-756; Logan, M. and Mohun, T. 1993. Induction of cardiac muscle differentiation in isolated animal pole explants of Xenopus laevis embryos. Development 118: 865-875; Marvin, M. J., Di Rocco, G., Gardiner, A., Bush, S. M., and Lassar, A. B. 2001. Inhibition of Wnt activity induces heart formation from posterior mesoderm. Genes & Dev. 15: 316-327; McGrew, L. L., Hoppler, S., and Moon, R. T. 1997. Wnt and FGF pathways cooperatively pattern anteroposterior neural ectoderm in Xenopus. Mech. Dev. 69: 105-114; Moon, R. T., Campbell, R. M., Christian, J. L., McGrew, L., Shih, J., and Fraser, S. 1993. Xwnt-5A: A maternal Wnt that affects morphogenetic movements after overexpression in embryos of Xenopus laevis. Development 119: 97-111; Moon, R. T., Brown, J. D., and Torres, M. 1997. WNTs modulate cell fate and behavior during vertebrate development. TIGS 13: 157-162; Nascone, N. and Mercola, M. 1995. An inductive role for endoderm in Xenopus cardiogenesis. Development 121: 515-523; Newman, C. S. and Krieg, P. A. 1998. tinman-related genes expressed during heart development in Xenopus. Dev. Gen. 22: 230-238; Newman, C. S., Reecy, J., Grow, M. W., Ni, K., Boettger, T., Kessel, M., Schwartz, R. J., and Krieg, P. A. 2000. Transient cardiac expression of the tinman-family homeobox gene XNkx2-10. Mech. Dev. 91: 369-373; Nieuwkoop, P. D. and Faber, J. 1994. Normal table of Xenopus laevis (Daudin): A systematical and chronological survey of the development from the fertilized egg till the end of metamorphosis. Garland, New York; Pearce, J. J. H., Penny, G., and Rossant, J. 1999. A mouse cerberus/DAN-related gene family. Dev. Biol. 209: 98-110; Pera, E. M. and De Robertis, E. M. 2000. A direct screen for secreted proteins in Xenopus embryos identifies distinct activities for the Wnt antagonists Crescent and Frzb-1. Mech. Dev. 96: 183-195; Pfeffer, P. L., De Robertis, E. M., and Izpisua-Belmonte, J.-C. 1997. Crescent, a novel chick gene encoding a Frizzled-like cysteine-rich domain, is expressed in anterior regions during early embryogenesis. J. Dev. Biol. 41: 449-458; Piccolo, S., Agius, E., Leyns, L., Bhattacharyya, S., Grunz, H., Bouwmeester, T., and De Robertis, E. M. 1999. The head inducer Cerberus is a multifunctional antagonist of Nodal, BMP and Wnt signals. Nature 397: 707-710; Salic, A. N., Kroll, K. L., Evans, L. M., and Kirschner, M. W. 1997. Sizzled: A secreted Xwnt8 antagonist expressed in the ventral marginal zone of Xenopus embryos. Development 124: 4739-4748; Sasai, Y., Lu, B., Steinbesser, H., Geissert, D., Gont, L. K., and De Robertis, E. M. 1994. Xenopus chordin: A novel dorsalizing factor activated by organizer-specific homeobox genes. Cell 79: 779-790; Sasai, Y., Lu, B., Piccolo, S., and De Robertis, E. M. 1996. Endoderm induction by the organizer-secreted factors chordin and noggin in Xenopus animal caps. EMBO J. 15: 4547-4555; Sater, A. K. and Jacobson, A. G. 1989. The specification of heart mesoderm occurs during gastrulation in Xenopus laevis. Development 105: 821-830; - - - . 1990. The role of the dorsal lip in the induction of heart mesoderm in Xenopus laevis. Development 108: 461-470; Schlange, T., Andree, B., Arnold, H. H., and Brand, T. 2000. BMP2 is required for early heart development during a distinct time period. Mech. Dev. 91: 259-270; Schneider, V. A. and Mercola, M. 1999. Spatially distinct head and heart inducers within the Xenopus organizer region. Curr. Biol. 9: 800-809; Schultheiss, T. M., Xydas, S., and Lassar, A. B. 1995. Induction of avian cardiac myogenesis by anterior endoderm. Development 121: 4203-4214; Schultheiss, T. M., Burch, J. B. E., and Lassar, A. B. 1997. A role for bone morphogenetic proteins in the induction of cardiac myogenesis. Genes & Dev. 11: 451-462; Shawlot, W., Deng, J. M., and Behringer, R. R. 1998. Expression of the mouse cerberus-related gene, Cerr1, suggests a role in anterior neural induction and somitogenesis. Proc. Natl. Acad. Sci. 95: 6198-6203; Sheldahl, L. C., Park, M., Malbon, C. C., and Moon, R. T. 1999. Protein kinase C is differentially stimulated by Wnt and Frizzled homologs in a G-protein-dependent manner. Curr. Biol 9: 695-698; Shi, Y., Katsev, S., Cai, C., and Evans, S. 2000. BMP is required for heart formation in vertebrates. Dev. Biol. 224: 226-237; Shibata, M., Ono, H., Hikasa, H., Shinga, J., and Taira, M. 2000. Xenopus crescent encoding a Frizzled-like domain is expressed in the Spemann organizer and pronephros. Mech. Dev. 96: 243-246; Simpson, E. H., Johnson, D. K., Hunsicker, P., Suffolk, R., Jordan, S. A., and Jackson, I. J. 1999. The mouse Cer1 (Cerberus related or homologue) gene is not required for anterior pattern formation. Dev. Biol. 213: 202-206; Smith, W. C. and Harland, R. M. 1991. Injected Xwnt-8 RNA acts early in Xenopus embryos to promote formation of a vegetal dorsalizing center. Cell 67: 753-765; Sokol, S., Christian, J. L., Moon, R. T., and Melton, D. A. 1991. Injected Wnt RNA induces a complete body axis in Xenopus embryos. Cell 67: 741-752; Song, J. and Slack, J. M. W. 1994. Spatial and temporal expression of basic fibroblast growth factor (FGF-2) mRNA and protein in early Xenopus development. Mech. Dev. 48: 141-151; Sugi, Y. and Lough, J. 1994. Anterior endoderm is a specific effector of terminal cardiac myocyte differentiation of cells from the embryonic heart forming region. Dev. Dyn. 200: 155-162; Suzuki, A., Nishimatsu, S., Murakami, K., and Ueno, N. 1993. Differential expression of Xenopus BMPs in early embryos and tissues. Zool. Sci. 10: 175-178; Tannahill, D., Isaacs, H. V., Close, M. J., Peters, G., and Slack, J. M. W. 1992. Developmental expression of the Xenopus int-2 (FGF-3) gene: Activation by mesodermal and neural induction. Development 115: 695-702; Tonissen, K. F., Drysdale, T. A., Lints, T. J., Harvey, R. P., and Krieg, P. A. 1994. XNkx-2.5, a Xenopus gene related to Nkx-2.5 and tinman: Evidence for a conserved role in cardiac development. Dev. Biol. 162: 325-328; Wang, S., Krinks, M., Lin, K., Luyten, F. P., and Moos, M., Jr. 1997a. Frzb, a secreted protein expressed in the Spemann organizer, binds and inhibits Wnt-8. Cell 88: 757-766; Wang, S., Krinks, M., and Moos, M. 1997b. Frzb-1, an antagonist of Wnt-1 and Wnt-8, does not block signaling by Wnts-3A, -5A or -11. Biochem. Biophys. Res. Comm. 236: 502-504; Wu, X., Golden, K., and Bodmer, R. 1995. Heart development in Drosophila requires the segment polarity gene wingless. Dev. Biol. 169: 619-628; Xu, Q., D'Amore, P. A., and Sokol, S. Y. 1998. Functional and biochemical interactions of Wnts with FrzA, a secreted Wnt antagonist. Development 125: 4767-4776; Yamagishi, T., Nishimatsu, S., Nomura, S., Asashima, M., Murakami, K., and Ueno, N. 1995. Expression of BMP-2, 4 genes during early development in Xenopus. Zool. Sci. 12: 355-358; Yatskievych, T. A., Ladd, A. N., and Antin, P. B. 1997. Induction of cardiac myogenesis in avian pregastrula epiblast: The role of the hypoblast and activin. Development 124: 2561-2570; Yokouchi, Y., Vogan, K. J., Pearse, R. V., and Tabin, C. J. 1999. Antagonistic signaling by Caronte, a novel Cerberus-related gene, establishes left-right asymmetric gene expression. Cell 98: 573-583; Zhu, L., Marvin, M. J., Gardiner, A., Lassar, A. B., Mercola, M., Stern, C. D., and Levin, M. 1999. Cerberus regulates left-right asymmetry of the embryonic head and heart. Curr. Biol. 9: 931-938; and Zimmerman, L. B., De Jesus-Escobar, J. M., and Harland, R. M. 1996. The Spemann organizer signal noggin binds and inactivates bone morphogenetic protein 4. Cell 86: 599-606.

### Example 3

### Inhibition of Wnt Activity Induces Heart Formation from Posterior Mesoderm

In the chick, heart mesoderm is induced by signals from the anterior endoderm. Although BMP-2 is expressed in the anterior endoderm, BMP activity is necessary but not sufficient for heart formation. Previous work from our lab has suggested that one or more additional factors from anterior endoderm are required. Crescent is a Frizzled-related protein that inhibits Wnt-8c and is expressed in anterior endoderm during gastrulation. At the same stages, expression of Wnt-3a and Wnt-8c is restricted to the primitive streak and posterior lateral plate, and is absent from the anterior region where crescent is expressed. Posterior lateral plate mesoderm normally forms blood, but coculture of this tissue with anterior endoderm or infection with RCAS-crescent induces formation of beating heart muscle and represses formation of blood. Dkk-1, a Wnt inhibitor of a different protein family, similarly induces heart-specific gene expression in posterior lateral plate mesoderm. Furthermore, we have found that ectopic Wnt signals can repress heart formation from anterior mesoderm in vitro and in vivo and that forced expression of either Wnt-3a or Wnt-8c can promote development of primitive erythrocytes from the precardiac region. We conclude that inhibition of Wnt signaling promotes heart formation in the anterior lateral mesoderm, whereas active Wnt signaling in the posterior lateral mesoderm promotes blood development.

Crescent is a Wnt-8c Antagonist Expressed in Anterior Endoderm

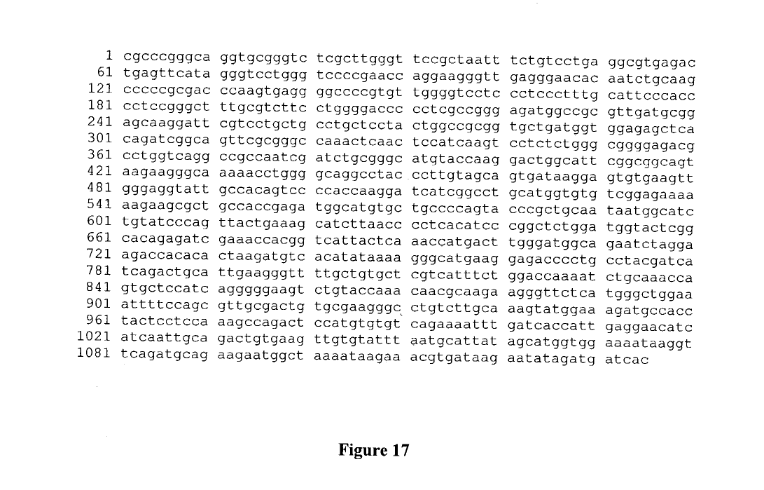

To search for signaling molecules in anterior endoderm that might be involved in heart induction, we used a suppression PCR-based cloning method (Diatchenko et al. 1996) to identify transcripts that are expressed in the anterior endoderm but are absent from the posterior primitive streak (PPS) in stage 5-6 chick embryos. A fragment of crescent (Pfeffer et al. 1997), a member of the FrzB class of Wnt antagonists (Leyns et al. 1997; Wang et al. 1997), was encoded by 2% of the subtracted clones. Crescent mRNA is abundant in the anterior hypoblast and anterior definitive endoderm from stage 2 to stage 6. At stage 5-6, crescent is expressed in prechordal mesendoderm as well, but at stage 6-7, its expression begins to decline in the endoderm underlying the presumptive heart and head mesoderm (FIG. 11, panels A-C; Pfeffer et al. 1997). Previous work has indicated that heart-inducing activity is present in both medial and lateral regions of stage 3-6 anterior mesoendoderm (Schultheiss et al. 1995, 1997), two regions of the embryo that express crescent transcripts (FIG. 11, panels A-C). In contrast, Wnt-8c is expressed in the primitive streak and in adjacent ectodermal cells at high levels and in the migrating posterior lateral plate (PLP) mesoderm at a relatively lower level (FIG. 11, panels D-F; Hume and Dodd 1993). In addition, Wnt-3a is expressed in the primitive streak from stage 3 (FIG. 11, panels G-I). Thus, crescent and Wnt expression domains are complementary, with crescent in the anterior and Wnt-8c and Wnt-3a in primitive streak and posterior tissues.

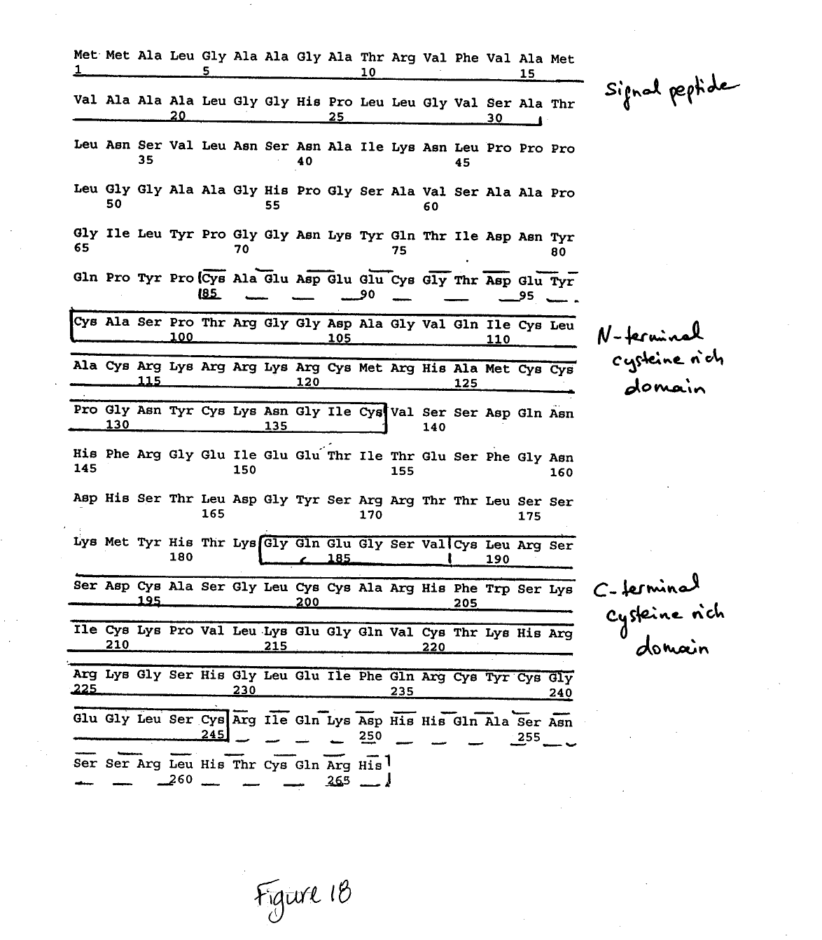

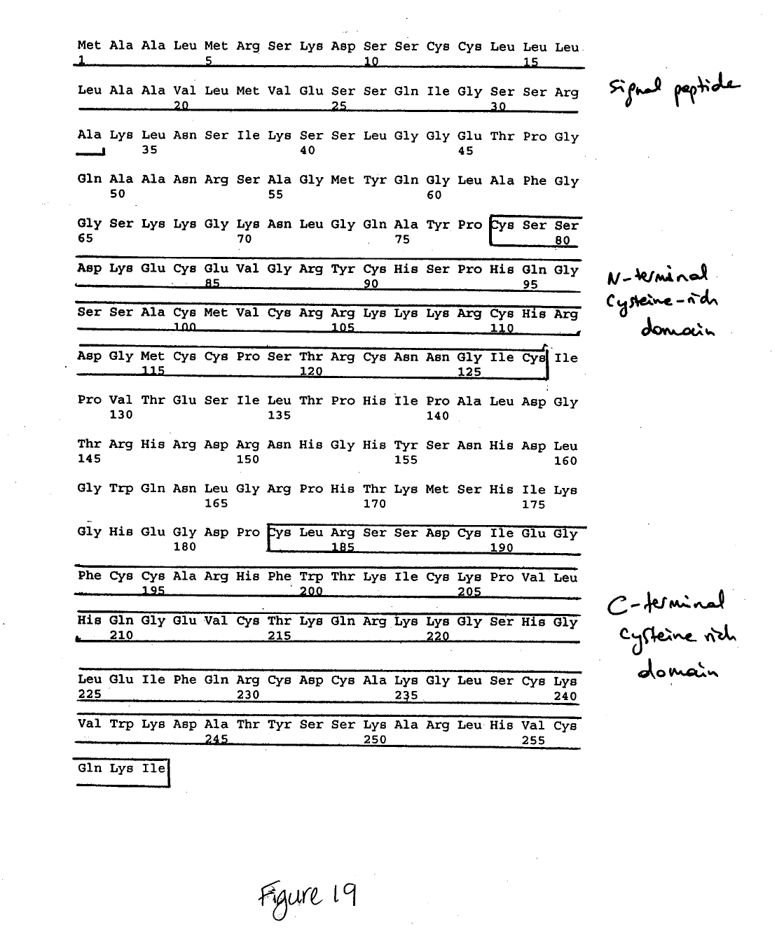

To test whether crescent can antagonize Wnt activity, we examined the effect of ectopic crescent expression in injected Xenopus embryos. As with other FrzB-related Wnt antagonists (Leyns et al. 1997; Salic et al. 1997; Wang et al. 1997; Deardorff et al. 1998; Xu et al. 1998; Itoh and Sokol 1999), injection of crescent RNA into the marginal zone of one cell of a two-cell Xenopus embryo enlarged anterior tissues and inhibited posterior extension (FIG. 12A). To directly address whether crescent is a Wnt antagonist, we examined whether crescent could block Wnt-induced expression of the homeobox gene siamois in Xenopus animal caps. Animal caps cut from embryos injected with chick Wnt-8c RNA expressed siamois (FIG. 12B, lane 4). Co-injection of crescent RNA at a sixfold molar ratio to Wnt-8c abolished this response (FIG. 12B, lane 5).

Injection of Wnt-3a RNA also induced expression of siamois in animal caps (FIG. 12B, lane 6). However, in this case, crescent co-injection could only partially dampen induction of siamois by Wnt-3a, reducing its expression threefold in response to a 120:1 molar excess of crescent to Wnt-3a RNA (FIG. 12B, lane 7). Although we do not know the relative steady-state levels of proteins produced by these injected RNAs, these results suggest that crescent is a potent inhibitor of Wnt-8c and a significantly weaker antagonist of Wnt-3a. Furthermore, these results suggest that the anterior expression of crescent and posterior expression of Wnt-8c and Wnt-3a in gastrula stage chick embryos combine to produce a gradient of Wnt activity, with lower levels of Wnt signaling in the anterior and higher levels in the posterior regions of the embryo.

Anterior Endoderm Induces Heart Muscle from Posterior Mesoderm and Primitive Streak

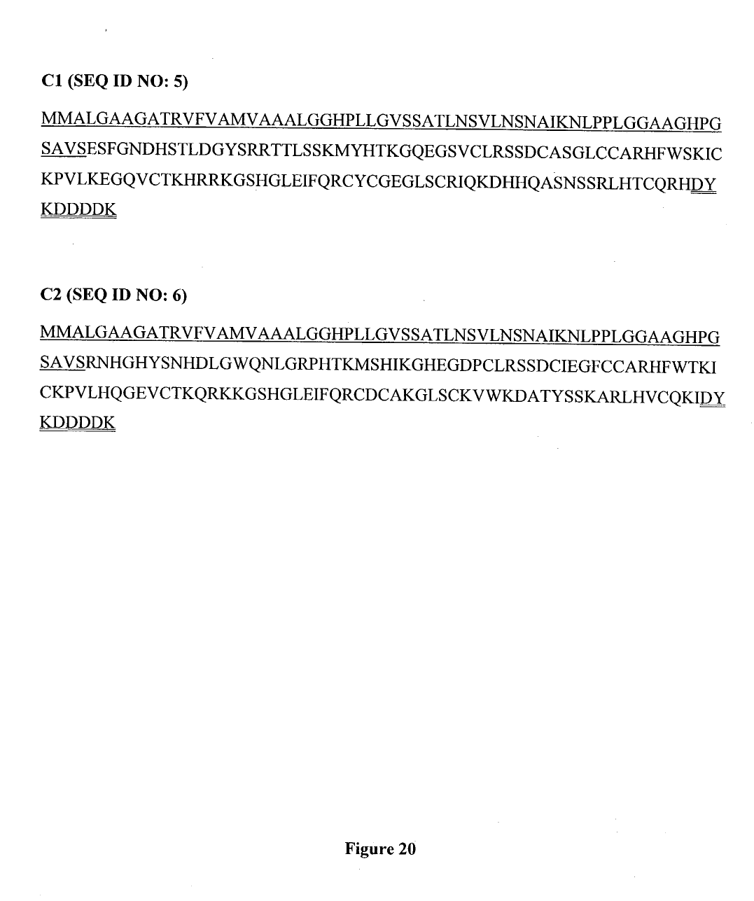

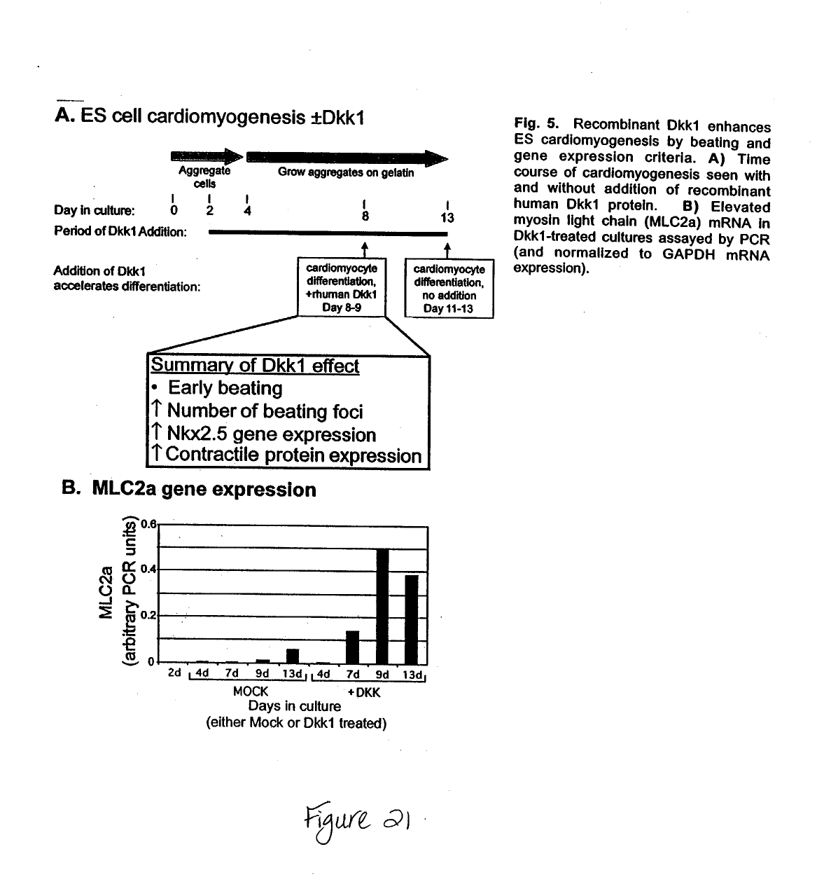

This laboratory previously demonstrated that anterior endoderm can induce stage 3-6 PPS to form heart muscle (Schultheiss et al. 1995). Here we show that anterior endoderm has a similar effect on stage 4⁺-6 posterior lateral plate (PLP) mesoderm. PLP mesoderm is a developmentally more advanced target tissue than PPS. This tissue contains cells that are fated to become solely mesodermal derivatives and lacks the epiblast layer present in primitive streak explants. Explants of either chick PLP mesoderm or PPS tissue failed to express any cardiac markers when cultured alone (FIG. 13, lanes 1, 3). In contrast, cocultures of these chick posterior tissues with quail anterior lateral mesendoderm from the precardiac region displayed robust expression of both chick and quail Nkx-2.5, ventricular myosin heavy chain (vMHC), and atrial myosin heavy chain (aMHC; FIG. 13, lanes 2, 4). Restriction fragment polymorphisms between the chick and quail genes were used to identify the species of the PCR products. Cells in both the PLP mesoderm and the PPS were responsive to the heart-inducing activity of the anterior endoderm. These findings indicate that anterior endoderm contains one or more signals that can induce cardiogenesis in either PPS tissue or PLP mesoderm, neither of which normally gives rise to heart.

Crescent or Dkk-1 Expression Converts Posterior Mesoderm to Heart Muscle

As crescent is expressed in anterior endoderm at approximately the stage expected for a heart-inducing factor, we investigated whether this Wnt inhibitor could induce the formation of heart muscle in explanted gastrula-stage posterior tissues. We made a replication-competent RCAS-crescent retrovirus and examined whether viral crescent expression can substitute for anterior endoderm in the cardiac induction assay. Explants of either PLP-mesoderm or PPS were infected with RCAS viruses encoding either crescent (RCAS-crescent) or alkaline phosphatase (RCAS-AP).

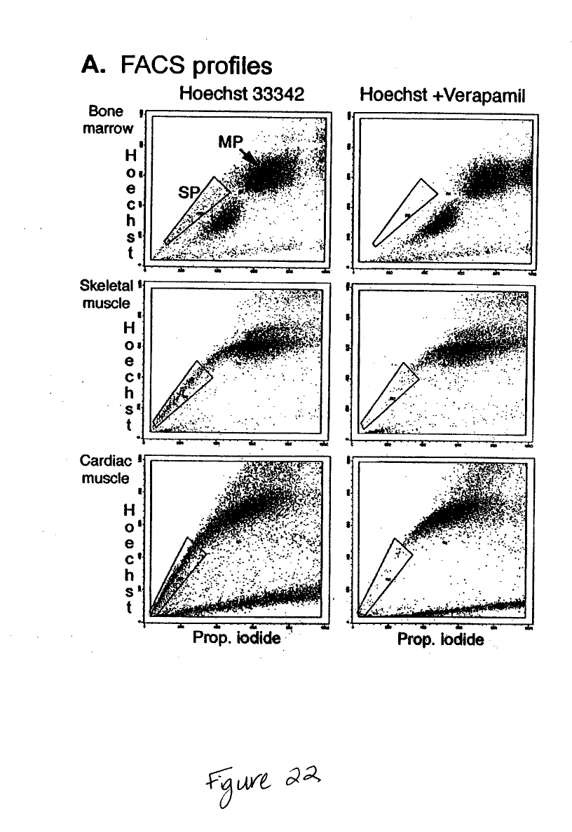

RCAS-AP infected explants of PLP mesoderm expressed the primitive erythrocyte marker, β-globin, and lacked cardiac gene expression (FIG. 14A, lane 1). In contrast, PLP mesoderm explants infected with RCAS-crescent expressed numerous heart markers including Nkx-2.5, vMHC, aMHC, GATA-4, and cardiac myosin heavy chain-1 (CMHC1) and began to beat rhythmically within 48 h of infection (FIG. 14A, lane 2). CMHC1 is a myosin isoform that is expressed exclusively within the heart (Croissant et al. 2000). As found for heart induction by endoderm (Schultheiss et al. 1995), RCAS-crescent reduced the expression of β-globin in explants of PLP mesoderm. These results are summarized in Table 1. Like the PLP mesodermal explants, PPS explants formed β-globin-expressing cells when infected with RCAS-AP (FIG. 14A, lane 3). However, in contrast to the strong cardiogenic response of PLP mesoderm to ectopic crescent, PPS explants showed only occasional weak induction of Nkx-2.5 yet no detectable expression of myosin or beating in response to RCAS-crescent infection (FIG. 14A, lane 4).

Although signals from the anterior endoderm can induce a cardiogenic response in both PPS and PLP mesoderm, crescent administration elicited cardiogenesis only in PLP mesoderm. These findings suggest that the signaling requirements necessary for heart induction differ between PLP mesoderm and PPS. PPS explants contain both the ectodermal and mesodermal layers of the streak, whereas PLP explants contain only mesoderm. The streak ectoderm showed the highest concentration of mRNA for both Wnt-3a and Wnt-8c by in situ hybridization (FIG. 11, panels F, I).

Accordingly, PPS expressed higher levels of Wnt-8c and Wnt-3a than PLP mesoderm at the time of dissection (FIG. 14B, cf. lanes 1 and 5). Furthermore, during in vitro culture of these tissues, expression of Wnt-8c and Wnt-3a declined to a greater extent in the PLP mesoderm than in PPS (FIG. 14B). The higher level and longer duration of Wnt-3a and Wnt-8c expression in PPS raised the possibility that signaling by these Wnt family members may prevent the induction of cardiac gene expression in PPS by ectopic Wnt antagonists.

Because PPS contains considerably more Wnt-3a mRNA than does PLP-mesoderm, and crescent is a relatively weak antagonist of this Wnt family member (FIG. 12B), we wondered if higher levels of Wnt-3a in the PPS could be blocking the cardiogenic effects of crescent in this tissue.

To explore this possibility, we evaluated whether expression of Dkk-1, another class of Wnt antagonist that inhibits both Wnt-8 and Wnt-3a signals (Kazanskaya et al. 2000; Krupnik et al. 2000), could activate cardiogenesis in either PLP-mesoderm or PPS tissues. COS cells transiently transfected with a plasmid encoding Xenopus Dkk-1 induced both Nkx2.5 and CMHC1 in cocultured PLP mesoderm (FIG. 14C, lane 2). In contrast to PLP mesoderm, cells of the posterior primitive streak failed to activate cardiac gene expression in response to Dkk-1 (FIG. 14C, lane 4). Under the same conditions, COS cells expressing crescent also induced Nkx-2.5 and CMHC1 expression in PLP mesoderm (FIG. 14C, lane 6) but not in PPS tissue (FIG. 14C, lane 8). Because both crescent and Dkk-l can induce cardiac gene expression in PLP mesoderm but not in PPS, it seems most likely that repression of Wnt-8c and Wnt-3a activity is sufficient to induce cardiogenesis in the PLP mesoderm but not in the PPS.

Table 1. Effect of RCAS-Crescent Infection on Gene Expression in Posterior Lateral Plate Mesoderm Explants.

| | Expression of Markers | | | | |
|---|---|---|---|---|---|
| Markers | n | Increase | No Change | Decrease | Not Expressed |
| Nkx | 17 | 94% | 6% | 0% | 0% |
| vMHC | 17 | 82% | 6% | 0% | 12% |
| CMHCl | 17 | 82% | 6% | 0% | 12% |
| aMHC | 17 | 88% | 12% | 0% | 0% |
| GATA-4 | 17 | 71% | 29% | 0% | 0% |
| Beating | 23 | 78% | 0 | 0% | 22% |
| Globin | 17 | 0 | 18% | 53% | 29% |

Percentage of posterior lateral plate explants that showed an increase or decrease in the expression of various marker genes (relative to GAPDH levels) on infection with RCAS-crescent, as compared to a paired control explant from the same embryo that was infected with RCAS-AP. Not expressed indicates that neither explants infected with RCAS-AP nor with RCAS-crescent expressed any detectable level of the gene indicated. No change indicates that background levels of the gene indicated were detected, but that these were identical in the control and experimental explant.

Ectopic Expression of Wnts Blocks Cardiogenesis from Precardiac Mesoderm

As inhibition of Wnt signaling can induce cardiogenesis in the PLP mesoderm, we hypothesized that expression of Wnt signals in the heart field would have the opposite effect. To address this issue, we examined whether ectopic expression of Wnt-3a in the presumptive heart field affects the expression of Nkx-2.5 in vivo. Embryos in which a pellet of chick embryo fibroblasts infected with RCAS-Wnt-3a (Kengaku et al. 1998) was implanted showed a marked decrease in the expression of Nkx-2.5 on the experimental side (FIGS. 15A, B). Contralateral control cell pellets did not affect Nkx-2.5 expression (FIGS. 15A, B). Implantation of cells expressing Wnt-1 similarly extinguished endogenous Nkx-2.5 expression in the presumptive heart field (data not shown). These results indicate that Wnt family members can suppress Nkx-2.5 gene expression in developing embryos. However, these in vivo experiments affected all three germ layers. The RCAS-Wnt-3a infected cells distorted the head of the embryo (FIG. 15B), and the neural plate was considerably expanded in some embryos implanted with Wnt-1 or Wnt-3a pellets (data not shown). Therefore, it was unclear whether repression of Nkx-2.5 gene expression by Wnt signals reflected a direct effect on precardiac mesoderm or a secondary effect because of the expansion of the neural plate, which is known to express inhibitors of cardiogenesis (Jacobson 1960; Climent et al. 1995; Schultheiss et al. 1997; Raffin et al. 2000).

To investigate whether Wnt signals can directly modulate cardiac gene expression in mesoderm, we infected explants of stage 5 presumptive heart mesoderm with either RCAS-Wnt-3a or RCAS-Wnt-8c. Heart mesoderm was cultured in serum-free medium containing 200 ng/mL BMP-4. Inclusion of BMP-4 in the medium supported robust cardiac differentiation from control precardiac mesoderm but was not strictly required for cardiogenesis (data not shown).

Infection of presumptive heart mesoderm with either RCAS-Wnt-3a or RCAS-Wnt-8c inhibited beating of the explants and reduced the expression of cardiac-specific genes in 100% (n=7) or 76% (n=17) of the infected explants, respectively (FIG. 15C).

These results indicate that ectopic expression of Wnt-3a and Wnt-8c, which are both expressed in cells of the primitive streak, can inhibit cardiac gene expression by a direct effect on mesoderm.

Wnt Signals Promote Erythrocyte Development from Precardiac Mesoderm

Primitive erythrocytes originate in the yolk sac blood islands that are derived from posterior primitive streak and posterior lateral plate (Rosenquist 1966; Robb 1997; Dieterlen-Lievre 1998; Palis et al. 1999). Infection of stage 5 precardiac mesoderm with either RCAS-Wnt-3a or RCAS-Wnt-8c promoted expression of the primitive erythrocyte marker β-globin (Minie et al. 1992) in 43% (n=7) or 29% (n=17) of infected explants, respectively (FIG. 15C, lanes 2, 4). In contrast, presumptive heart mesoderm from stage 5 embryos failed to express β-globin when infected with control RCAS viruses in 100% of such explants (n=24; FIG. 15C, lanes 1, 3). This result is consistent with our finding that crescent administration to PLP mesoderm abolishes globin expression in this tissue (FIG. 14A, lane 2) and indicates that Wnt signaling is necessary to promote formation of embryonic blood cells. Furthermore, it demonstrates that Wnts and Wnt inhibitors have reciprocal roles in A-P patterning of lateral mesoderm, with inhibition of Wnt signaling promoting an anterior mesodermal fate and high levels of Wnt signaling promoting a posterior mesodermal fate.

Materials & Methods

Subtraction

First- and second-strand cDNA synthesis (Life Technologies) was carried out on the polyA+ fraction of 0.3-0.5 μg of total RNA (OligoTex, QIAGEN). The cDNA was digested with Rsa1 and ligated to annealed primer pairs 2Rsa24: AGCACTCTC CAGGTACTCCACGGT (SEQ ID NO: 19) and 2Rsa10: ACCGTGGAGT (SEQ ID NO: 20), modified from Braun et al. (1994). cDNA was amplified by PCR: 72° C. for 5 min; 28 cycles 93° C. for 30 sec, 68° C. for 30 sec, 72° C. for 3 min. cDNA was digested with RsaI.

Anterior lateral plate endoderm and posterior primitive streak cDNA were used as target and driver, respectively, in the PCR-Select Subtraction Kit (Clontech). Target concentration was 1.7 ng/5 μL, and the driver/target ratio was 68:1 in the first hybridization and 90:1 in the second.

Subtracted clones were amplified at 64° C. for 27 cycles. The subtracted endoderm was cloned into Bluescript SK+. Duplicate filters containing the subtracted endoderm plasmid library were screened with the library itself as a positive probe and with PPS driver plus PPS subtracted with endoderm as the negative probe. Clones that hybridized strongly or moderately to the positive probe and did not hybridize with the negative probe were sequenced.

RCAS Virus

Crescent was amplified from cDNA from stage 4 anterior endoderm with primers TTTTTTCCATGGGGGCTGCGAGCACGGAGA (SEQ ID NO: 21) and TTTTTAAAGCTTTCAGACCTTCCTGC CGGCCTGTT (SEQ ID NO: 22). A PCR product encoding crescent was cut with Nco1 and HindIII and cloned into the vector SLAX-13 (Morgan and Fekete 1996), then subcloned into the Cla1 site of RCAS(B). Chick Wnt-8c was amplified from pGEM cWnt-8c with the primers AGTTCCACGCTCGGTCTC CCATGAGAGGCAGCACCTTC (SEQ ID NO: 23) and TTGTTAGCAAGCTT CTATCTCCTGTGGCCTTTGT (SEQ ID NO: 24) and was cut with Bsa1 and HindIII. The fragments were cloned into the Nco and HindIII sites of SLAX-13, and from there into the Cla1 site of RCAS(B). All viruses were produced in line 0 chick dermal fibroblasts as described in Maroto et al. (1997).

Explant Cultures

Eggs were incubated to the given stage (Hamburger and Hamilton 1951), and tissues were dissected with tungsten needles in Tyrodes solution using 1% agar dishes as a base. Serum-free medium containing insulin, transferrin, and selenium was adapted from Stern and Hauschka (1995) and supplemented with 2% chick embryo extract (Life Technologies). Virally infected explants were incubated on ice with viral supernatant diluted 1:1 with culture medium for 1-2 h, then cultured overnight in a sandwich of 35% collagen pads and overlaid with the above concentration of viral supernatant and medium containing 8 μg/mL polybrene. The following day, 0.25 mL of culture medium was added to each well. Anterior endoderm and COS cell cocultures were carried out on 2-μ pore size Nucleopore filters floating on culture medium. Similar results were obtained for anterior endoderm induction in collagen gels.

Posterior primitive streak explants were cut from 80%-100% streak length, and posterior lateral plate mesoderm explants were cut from 75%-100% streak length. PLP mesoderm was carefully scraped off the ectoderm after removal of the endoderm. COS cells were transfected with Fugene (Roche). The plasmids transfected were: pCS2⁺-nβ-gal, pCS2⁺-crescent, and pCMV2-XDkk-1 (a generous gift of Dr. Christoph Niehrs, DKFZ, Heidelberg, Germany). BMP-4 (R&D Systems) was added to the viral supernatant at 200 ng/mL for overnight incubation and at 20 ng/mL to the culture medium. Cultures were grown for 64 h unless otherwise noted.

RT-PCR

RT-PCR was carried out as in Schultheiss et al. (1995). Additional primers were as follows: aMHC (Yutzey et al. 1994), CCGCACCACAGAAGACCAGAT (SEQ ID NO: 25) and GGAGGAGCACTTG GCATTGAC (SEQ ID NO: 26); CMHC1 (Croissant et al. 2000), TGACCAGGGTG GAGAAAAG (SEQ ID NO: 27) and TTGTCCTCTGGGATTGCACCTG (SEQ ID NO: 28); GAPDH (glyceraldehyde 3-phosphate dehydrogenase), Nkx-2.5, and vMHC were digested as described in Schultheiss et al. (1995). aMHC products were cut with AvaII, such that the chick aMHC PCR product gave two bands at 299 and 190 bp, whereas quail aMHC gave bands at ˜185, 179, and 125 bp. The 299-bp chick product and 125-bp quail product are shown here. The aMHC primers amplified chick cDNA with greater affinity than quail.

New Culture and In Situ Hybridization

The albumen was removed from stage 3-4 eggs. A 2.5-cm Fisher P5 filter paper ring was placed on top of the embryo, and the yolk was gently submerged in Pannett-Compton solution. The vitelline membrane was cut around the outside of the paper ring while the yolk was submerged, and the paper and embryo assembly was inverted, washed, and placed in a dish containing 0.3% glucose, egg white, and agar as described by Sundin and Eichele (1992). Pellets of RatB1A cells or RCAS-infected fibroblasts were placed in the heart-forming region of the embryo and cultured until the stages indicated. Embryos were fixed in 4% paraformaldehyde in pH 7.4 PBS and processed for in situ hybridization (Wilkinson 1993).

References: Andree, B., Duprez, D., Vorbusch, B., Arnold, H., and Brand, T. 1998. BMP-2 induces ectopic expression of cardiac lineage markers and interferes with somite formation in chicken embryos. Mech. Dev. 70: 119-131; Antin, P. B., Taylor, R. G., and Yatskievych, T. 1994. Precardiac mesoderm is specified during gastrulation in quail. Dev. Dyn. 200: 144-154; Azpiazu, N. and Frasch, M. 1993. tinman and bagpipe: Two homeo box genes that determine cell fates in the dorsal mesoderm of Drosophila. Genes & Dev. 7: 1325-1340; Baranski, M., Berdougo, E., Sandler, J. S., Darnell, D. K., and Burrus, L. W. 2000. The dynamic expression pattern of frzb-1 suggests multiple roles in chick development. Dev. Biol. 217: 25-41; Belaoussoff, M., Farrington, S. M., and Baron, M. H. 1998. Hematopoietic induction and respecification of A-P identity by visceral endoderm signaling in the mouse. Development 125: 5009-5018; Bodmer, R. 1993. The gene tinman is required for specification of the heart and visceral muscles in Drosophila. Development 118: 719-729; Bouwmeester, T., Kim, S., Sasai, Y., Lu, B., and Robertis, E. M. D. 1996. Cerberus is a head-inducing secreted factor expressed in the anterior endoderm of Spemann's organizer. Nature 382: 595-601; Bradley, L., Sun, B., Collins-Racie, L., LaVallie, E., McCoy, J., and Sive, H. 2000. Different activities of the frizzled-related proteins frzb2 and sizzled2 during Xenopus anteroposterior patterning. Dev. Biol. 227: 118-132; Braun, B., Frieden, R., Lessnick, S., May, W., and Denny, C. 1994. Identification of target genes for the Ewing's sarcoma EWS/FLI fusion protein by representational difference analysis. Mol. Cell. Biol. 15: 4623-4630; Christian, J. and Moon, R. 1993. Interactions between Xwnt-8 and Spemann organizer signaling pathways generate dorsoventral pattern in the embryonic mesoderm of Xenopus. Genes & Dev. 7: 13-28; Climent, S., Sarasa, M., Villar, J. M., and Murillo-Ferrol, N. L. 1995. Neurogenic cells inhibit the differentiation of cardiogenic cells. Dev. Biol. 171: 130-148; Croissant, J. D., Carpenter, S., and Bader, D. 2000. Identification and genomic cloning of CMHC1: A unique myosin heavy chain expressed exclusively in the developing chicken heart. J. Biol. Chem. 275: 1944-1951; Dale, L., Howes, G., Prive, B., and Smith, J. 1992. Bone morphogenetic protein 4: A ventralizing factor in early Xenopus development. Development 115: 573-585; Deardorff, M. A., Tan, C., Conrad, L. J., and Klein, P. S. 1998. Frizzled-8 is expressed in the Spemann organizer and plays a role in early morphogenesis. Development 125: 2687-2700; Diatchenko, L., Lau, Y.-F., Campbell, A., Chenchik, A., Moqadam, F., Huang, B., Lukyanov, S., Lukanov, K., Gurskaya, N., Sverdlov, E. et al. 1996. Suppression subtractive hybridization: A method for generating differentially regulated or tissue-specific cDNA probes and libraries. Proc. Natl. Acad. Sci. 93: 6025-6030; Dieterlen-Lièvre, F. 1998. Hematopoiesis: Progenitors and their genetic program. Curr. Biol. 8: R727-R730; Eisenberg, C. A. and Eisenberg, L. M. 1999. WNT11 promotes cardiac tissue formation of early mesoderm. Dev. Dyn. 216: 45-58; Eisenberg, C. A., Gourdie, R. G., and Eisenberg, L. M. 1997. Wnt-11 is expressed in early avian mesoderm and required for the differentiation of the quail mesoderm cell line QCE-6. Development 124: 525-536; Fainsod, A., Steinbeisser, H., and De Robertis, E. M. 1994. On the function of BMP-4 in patterning the marginal zone of the Xenopus embryo. EMBO J 13: 5015-5025; Fu, Y., Yan, W., Mohun, T., and Evans, S. 1998. Vertebrate tinman homologues XNkx2-3 and XNkx2-5 are required for heart formation in a functionally redundant manner. Development 125: 4439-4449; Garcia-Martinez, V. and Schoenwolf, G. C. 1993. Primitive streak origin of the cardiovascular system in avian embryos. Dev. Biol. 159: 706-719; Glinka, A., Wu, W., Onichtchouk, D., Blumenstock, C., and Niehrs, C. 1997. Head induction by simultaneous repression of Bmp and Wnt signalling in Xenopus. Nature 389: 517-519; Glinka, A., Wu, W., Delius, H., Monaghan, A., Blumenstock, C., and Niehrs, C. 1998. Dickkopf-1 is a member of a new family of secreted proteins and functions in head induction. Nature 391: 357-362; Graff, J. M., Thies, R. S., Song, J. J., Celeste, A. J., and Melton, D. A. 1994. Studies with a Xenopus BMP receptor suggest that ventral mesoderm-inducing signals override dorsal signals in vivo. Cell 79: 169-179; Grow, M. and Krieg, P. 1998. Tinman function is essential for vertebrate heart development: Elimination of cardiac differentiation by dominant inhibitory mutants of the tinman-related genes, XNkx2-3 and XNkx2-5. Dev. Biol. 204: 187-196; Hamburger, V. and Hamilton, H. L. 1951. A series of normal stages in the development of the chick embryo. J. Morphol. 88: 49-92; Hoppler, S. and Moon, R. T. 1998. BMP-2/-4 and Wnt-8 cooperatively pattern the Xenopus mesoderm. Mech. Dev. 71: 119-129; Hoppler, S., Brown, J. D., and Moon, R. T. 1996. Expression of a dominant negative Wnt blocks induction of MyoD in Xenopus embryos. Genes & Dev. 10: 2805-2817; Hsu, D. R., Economides, A. N., Wang, X., Eimon, P. M., and Harland, R. M. 1998. The Xenopus dorsalizing factor Gremlin identifies a novel family of secreted proteins that antagonize BMP activities. Mol. Cell 1: 673-683; Hume, C. R. and Dodd, J. 1993. Cwnt-8C: A novel Wnt gene with a potential role in primitive streak formation and hindbrain organization. Development 119: 1147-1160; Inagaki, T., Garcia-Martinez, V., and Schoenwolf, G. C. 1993. Regulative ability of the prospective cardiogenic and vasculogenic areas of the primitive streak during avian gastrulation. Dev. Dyn. 197: 57-68; Itoh, K. and Sokol, S. Y. 1999. Axis determination by inhibition of Wnt signaling in Xenopus. Genes & Dev. 13: 2328-2336; Jacobson, A. G. 1960. Influences of ectoderm and endoderm on heart differentiation in the newt. Dev. Biol. 2: 138-154; Jones, C. M., Lyons, K. M., Lapan, P. M., Wright, C. V. E., and Hogan, B. M. L. 1992. DVR-4 (bone morphogenetic protein-4) as a posterior ventralizing factor in Xenopus mesoderm induction. Development 115: 639-647; Kazanskaya, O., Glinka, A., and Niehrs, C. 2000. The role of Xenopus dickkopf1 in prechordal plate specification and neural patterning. Development 127: 4981-4992; Kengaku, M., Capdevila, J., Rodriguez-Esteban, C., Pena, J. D. L., Johnson, R., Izpisua-Belmonte, J. C., and Tabin, C. J. 1998. Distinct WNT pathways regulating AER formation and dorsoventral polarity in the chick limb bud. Science 280: 1274-1277; Krupnik, V., Sharp, J., Jiang, C., Robison, K., Chickering, T., Amaravadi, L., Brown, D., Guyot, D., Mays, G., Leiby, K. et al. 2000. Functional and structural diversity of the human Dickkopf gene family. Gene 238: 301-313; Ladd, A., Yatskievych, T., and Antin, P. 1998. Regulation of avian cardiac morphogenesis by activin/TGFb and bone morphogenetic proteins. Dev. Biol. 204: 407-419; Leyns, L., Bouwmeester, T., Kim, S.-H., Piccolo, S., and De Robertis, E. 1997. Frzb-1 is a secreted antagonist of Wnt signaling expressed in the Spemann Organizer. Cell 88: 747-756; Lough, J., Barron, M., Brogley, M., Sugi, Y., Bolender, D. L., and Zhu, X. L. 1996. Combined BMP-2 and FGF-4, but neither factor alone, induces cardiogenesis in non-precardiac embryonic mesoderm. Dev. Biol. 178: 198-202; Maeno, M., Ong, R. C., Suzuki, A., Ueno, N., and Kung, H. F. 1994. A truncated bone morphogenetic protein 4 receptor alters the fate of ventral mesoderm to dorsal mesoderm: Roles of animal pole tissue in the development of ventral mesoderm. Proc. Natl Acad. Sci. 91: 10260-10264; Maroto, M., Reshef, R., Münsterberg, A. E., Koester, S., Goulding, M., and Lassar, A. B. 1997. Ectopic Pax-3 activates MyoD and Myf-5 expression in embryonic mesoderm and neural tissue. Cell 89: 139-148; Minie, M., Kimura, T., and Felsenfeld; G. 1992. The developmental switch in embryonic r-globin expression is correlated with erythroid lineage-specific differences in transcription factor levels. Development 115: 1149-1164; Monaghan, A. P., Kioschis, P., Wu, W., Zuniga, A., Bock, D., Poustka, A., Delius, H., and Niehrs, C. 1999. Dickkopf genes are co-ordinately expressed in mesodermal lineages. Mech. Dev. 87: 45-56; Montgomery, M. O., Litvin, J., Gonzalez-Sanchez, A., and Bader, D. 1994. Staging of commitment and differentiation of avian cardiac myocytes. Dev. Biol. 164: 63-71; Morgan, B. and Fekete, D. 1996. Manipulating gene expression with replication-competent retroviruses. In Methods in avian embryology (ed. M. Bronner-Fraser), pp. 185-218. Academic Press, San Diego, Calif.; Nascone, N. and Mercola, M. 1995. An inductive role for the endoderm in Xenopus cardiogenesis. Development 121: 515-523; Palis, J., Robertson, S., Kennedy, M., Wall, C., and Keller, G. 1999. Development of erythroid and myeloid progenitors in the yolk sac and embryo proper of the mouse. Development 126: 5073-5084; Park, M., Wu, X., Golden, K., Axelrod, J. D., and Bodmer, R. 1996. The wingless signaling pathway is directly involved in Drosophila heart development. Dev. Biol. 177: 104-116; Park, M., Lewis, C., Turbay, D., Chung, A., Chen, J.-N., Evans, S., Breitbart, R., Fishman, M., Izumo, S., and Bodmer, R. 1998. Differential rescue of visceral and cardiac defects in Drosophila by vertebrate tinman-related genes. Proc. Natl. Acad. Sci. 95: 9366-9371; Parr, B., Shea, M., Vassileva, G., and McMahon, A. 1993. Mouse Wnt genes exhibit discrete domains of expression in the early embryonic CNS and limb buds. Development 119: 247-261; Pera, E. M. and De Robertis, E. M. 2000. A direct screen for secreted proteins in Xenopus embryos identifies distinct activities for the Wnt antagonists Crescent and Frzb-1. Mech. Dev. 96: 183-195; Pfeffer, P., DeRobertis, E., and Izpisua-Belmonte, J.-C. 1997. Crescent, a novel chick gene encoding a Frizzled-like Cysteine-Rich Domain, is expressed in anterior regions during early embryogenesis. Int. J Dev. Biol. 41: 449-458; Piccolo, S., Agius, E., Leyns, L., Bhattacharyya, S., Grunz, H., Bouwmeester, T., and Robertis, E. D. 1999. The head inducer Cerberus is a multifunctional antagonis of Nodal, BMP and Wnt signals. Nature 397: 707-710; Raffin, M., Leong, L., Rones, M., Sparrow, D., Mohun, T., and Mercola, M. 2000. Subdivision of the cardiac Nkx-2.5 expression domain into myogenic and nonmyogenic compartments. Dev. Biol. 218: 326-340; Ranganayakulu, G., Elliott, D., Harvey, R., and Olson, E. 1998. Divergent roles for NK-2 class homeobox genes in cardiogenesis in flies and mice. Development 125: 3037-3048; Riefers, F., Walsh, E., Leger, S., Stainier, D., and Brand, M. 2000. Induction and differentiation of the zebrafish heart requires fibroblast growth factor 8 (fgf8/acerebellar). Development 127: 225-235; Robb, L. 1997. Hematopoiesis: Origin pinned down at last? Curr. Biol. 7: R1O-R12; Rodriguez-Esteban, C., Capdevila, J., Economides, A., Pascual, J., Ortiz, A., and Izpisua-Belmonte, J. 1999. The novel Cer-like protein Caronte mediates the establishment of embryonic left-right asymmetry. Nature 401: 243-251; Rosenquist, G. C. 1966. A radioautographic study of labelled grafts in the chick blastoderm. Carnegie Institute of Washington Contributions to Embryology 38: 71-110; Rosenquist, G. C. and DeHaan, R. L. 1966. Migration of Precardiac Cells in the Chick Embryo: A Radioautographic Study. Carnegie Inst. Washington Contrib. Embryol. 38: 111-121; Salic, A., Kroll, K., Evens, L., and Kirschner, M. 1997. Sizzled: A secreted Xwnt8 antagonist expressed in the ventral marginal zone of Xenopus embryos. Development 124: 4739-4748; Schlange, T., Andree, B., Arnold, H., and Brand, T. 2000. BMP2 is required for early heart development during a distinct time period. Mech. Dev. 91: 259-270; Schneider, V. and Mercola, M. 2001. Wnt antagonism initiates cardiogenesis in Xenopus laevis. Genes & Dev. 15: 304-315; Schoenwolf, G. C., Garcia-Martinez, V., and Dias, M. S. 1992. Mesoderm movement and fate during avian gastrulation and neurulation. Dev. Dyn. 193: 235-248; Schultheiss, T. M. and Lassar, A. B. 1997. Induction of chick cardiac myogenesis by bone morphogenetic proteins. Cold Spring Harbor Symp. Quant. Biol. 62: 413-419; Schultheiss, T. M., Xydas, S., and Lassar, A. B. 1995. Induction of avian cardiac myogenesis by anterior endoderm. Development 121: 4203-4214; Schultheiss, T., Burch, J., and Lassar, A. 1997. A role for bone morphogenetic proteins in the induction of cardiac myogenesis. Genes & Dev. 11: 451-462; Stern, H. and Hauschka, S. 1995. Neural tube and notochord promote in vitro myogenesis in single somite explants. Dev. Biol. 167: 87-103; Sugi, Y. and Lough, J. 1994. Anterior endoderm is a specific effector of terminal cardiac myocyte differentiation of cells from the embryonic heart forming region. Dev. Dyn. 200: 155-162; Sundin, O. and Eichele, G. 1992. An early marker of axial pattern in the chick embryo and its respecification by retinoic acid. Development 114: 841-852; Suzuki, A., Thies, R. S., Yamaji, N., Song, J. J., Wozney, J. M., Murakami, K., and Ueno, N. 1994. A truncated bone morphogenetic protein receptor affects dorsal-ventral patterning in the early Xenopus embryo. Proc. Natl Acad. Sci. 91: 10255-10259; Torres, M. A., Yang-Snyder, J. A., Purcell, S. M., DeMarais, A. A., McGrew, L. L., and Moon, R. T. 1996. Activities of the Wnt-1 class of secreted signaling factors are antagonized by the Wnt-5A class and by a dominant negative cadherin in early Xenopus development. J. Cell. Biol. 133: 1123-1137; Tzahor, E. and Lassar, A. B. 2001. Wnt signals from neural tube block ectopic cardiogenesis. Genes & Dev. 15: 255-260; Wang, S., Krinks, M., Lin, K., Luyten, F., and Moos, M. 1997. Frzb, a secreted protein expressed in the Spemann Organizer, binds and inhibits Wnt-8. Cell 88: 757-766; Wilkinson, D. G. 1993. In situ hybridization. In Essential developmental biology, a practical approach (ed. C. D. Stern and P. W. H. Holland), pp. 257-274. IRL Press, Oxford; Wu, X., Golden, K., and Bodmer, R. 1995. Heart development in Drosophila requires the segment polarity gene wingless. Dev. Biol. 169: 619-628; Xu, Q., D'Amore, P., and Sokol, S. 1998. Functional and biochemical interactions of Wnts with FrzA, a secreted Wnt antagonist. Development 125: 4767-4776; Yokouchi, Y., Vogan, K., Pearse, R. V., II, and Tabin, C. 1999. Antagonistic signaling by caronte, a novel cerberus-related gene, establishes left-right asymmetric gene expression. Cell 98: 573-583; Yutzey, K. E., Rhee, J. T., and Bader, D. 1994. Expression of the atrial-specific myosin heavy chain AMHC1 and the establishment of anteroposterior polarity in the developing chicken heart. Development 120: 871-883; and Zhu, L., Marvin, M., Gardiner, A., Lassar, A., Mercola, M., Stern, C., and Levin, M. 1999. Cerberus regulates left-right asymmetry of the embryonic head and heart. Curr. Biol. 9: 931-938.

### Example 4

### Preparation of Fragments of Dkk Proteins

To identify specific protein regions responsible for different signaling properties of Dkk1 and Dkk2, we analyzed constructs containing either the amino-terminal or the carboxy-terminal cysteine-rich domains of Dkk1, Dkk2, or Dkk3 (N1 and C1, N2 and C2, N3 and C3, respectively, FIGS. 16-19). To further investigate the role of the N-terminal domains in specifying the functional properties of Dkk1 and Dkk2, we generated chimeric Dkks (N1C2 and N2C 1), in which the N- and C-terminal domains of Dkk1 and Dkk2 were exchanged (FIGS. 18-20).

DNA constructs. pCS2-Dkk1-Flag, pCS2-Dkk2-Flag, and pCS2-Dkk3-Flag have been previously described (Krupnik, V. E., et al. 1999. Gene 238:301-313). Individual Dkk domain constructs, except for N2 and N2C 1, were generated by fusing the signal peptide of Dkk1 to the N-terminal (N1, N1C2) or C-terminal cysteine-rich domains (C1, C2, or C3) of Dkk1, Dkk2, or Dkk3, respectively. N2 and N2C1 contain the Dkk2 signal peptide fused to the N-terminal cysteine-rich region of Dkk2. The Ni construct was amplified from pCS2-Dkk1 using polymerase chain reaction (PCR) with the SP6 primer (Promega) and 5′-CCGCTCGAGCTAAGCGTAATCTGGAACATCGTATGGATACCCATCCAAGGTGCT-3′ (SEQ ID NO: 29), encoding a hemagglutinin tag. The PCR product was subcloned into pCS2 using EcoRI and XhoI sites. This construct was used in all studies except the analysis of protein expression levels, for which a Flag-tagged N1 construct was utilized. pCS2-N1-Flag was synthesized with Pfu 1 polymerase (Stratagene), using pCS2-Dkk1 as a template, and the primer 5′-CCATCACTGAAAGCTTTGAATTCGACTACAAGGAC GACGA-3′ (SEQ ID NO: 30), as described (Makarova, O., et al. 2000. BioTechniques 29:970-972).

The C1 construct was generated by ligating together EcoR1-Asp718-digested pCS2, HindIII-Asp718-digested C-terminal half of Dkk1, and an EcoR1-HindIII-digested DNA fragment derived from PCR of pCS2-Dkk1 with the SP6 primer and the oligonucleotide 5′-TCCAAGCTTACTGCAGAGCCTGG-3′ (SEQ ID NO: 31). The N2 construct was made by PCR using pCS2-Dkk2 as a template, the SP6 primer and the oligonucleotide 5′-GATGGTACTCGGCACAGAAGCTTGCG-3′ (SEQ ID NO: 32). The PCR product was digested with HindIII, and subcloned into pCS2-NI digested with HindIII to remove the N1 fragment. C2 was constructed by PCR amplifying the C-terminal half of Dkk2 from pCS2-Dkk2 with the T3 primer (Stratagene) and the oligonucleotide 5′-CGCAAGCTTAAACCACGGTCATTAC-3′ (SEQ ID NO: 33). The PCR fragment digested with HindIII and Asp718 was subcloned into pCS2-C1 digested with HindIII and Asp718 to remove C1. C3 was constructed by PCR of the C-terminal half of Dkk3 using the T3 primer, and the oligonucleotide 5′-CGCAAGCTTGGCCACCAGGGGCAGCA-3′ (SEQ ID NO: 34). This fragment was digested with HindIII and XbaI, and cloned into pCS2-C1 digested with HindIII and XbaI, to remove the C1 fragment. pCS2-N1C2 was constructed by PCR of the C-terminal half of Dkk2 using the T3 primer, and the primer used for construction of the C2 construct (see above). This fragment was digested with HindIII and Asp718, and ligated to pCS2-Dkk1 cut with HindIII and EcoRI, and Asp718-EcoRI digested pCS2. pCS2-N2C1 was constructed by PCR of the N-terminal half of Dkk2 using the SP6 primer and the primer used for construction of the N2 construct (see above). This fragment was digested with HindIII and EcoRI, and ligated to pCS2-Dkk1 cut with Asp718 and HindIII, and Asp718-EcoRI-digested pCS2.

Dkk1-GFP, Dkk2-GFP, C1-GFP, and C2-GFP were generated by PCR, using pCS2-Dkk-flag constructs as template, with the primer 5′-GGATCCTTGTCGTCGTGGCC-3′ (SEQ ID NO: 35), which contains a BamHI site, and the SP6 primer. These products were subcloned into pEGFP-1 (Clontech) using BamHI and EcoRI sites. The constructs were then digested with NotI, blunted, and EcoRI, and then subcloned into the pCS2 vector. pCS2-N2-GFP was constructed, using pCS2-Dkk2-GFP as a template, and the primer 5′-GGATGGTACTCGGCACCTCGAGGACTACAAGGACGACG-3′ (SEQ ID NO: 36) as described (Mao, B., et al., 2001b. Nature 411:321-325). All constructs were verified by DNA sequencing. pSia-Luc, pCS2-LRP6, pSP64T-XWnt8, and pSP64T-tBMPR (tBR) plasmids have been previously described (Christian, J. L., et al., 1991. Development 111: 1045-1055; Fan, M. J., et al., 1998. Proc. Natl. Acad. Sci. USA 95:5626-5631; Graff, J. M., et al., 1994. Cell 79:169-179; Tamai, K., et al., 2000. Nature 407:530-535).

Cell culture, transfections, and fluorescent microscopy. Human embryonic kidney 293T (HEK293T) cells were cultured in IX Dulbecco's Modified Eagle Medium (DMEM) (Gibco/Invitrogen) supplemented with 10% fetal calf serum (Gibco/Invitrogen) and 5 μg/ml gentamicin (Sigma). Cells were transiently transfected with 5 μg of each Dkk-GFP construct using the calcium phosphate method as described (Chen, C., and Okayama, H. 1987. Mol. Cell. Biol. 7:2745-2752). Cell culture medium containing GFP-tagged forms of Dkks was collected 48 hours after transfection, and was added to glass coverslips seeded with HEK293T cells transfected earlier with 10 μg of pCS2-LRP6 or the control pCS2 vector, for one hour at 37° C. Coverslips were then washed 2X with PBS, fixed in 4% paraformaldehyde, washed 2X with PBS, and assessed by fluorescence microscopy.

### Example 5

### Dkk1 Accelerates and Enhances Cardiac Differentiation of ES Cells

Full length human Dkk1 (SEQ ID NO: 2) was expressed in host cells and incubated with murine embryonic stem (ES) cells. ES cells were lightly trypsinized, re-plated on petri plastic and cultured for 4 days. During this time, the cells cluster and the mesodermal marker brachyury becomes expressed. Cells were then trypsinized lightly and transferred to gelatin-coated plates with or without recombinant Dkk1 and cultured at 37° C. Cardiomyocyte differentiation of ES cells in the absence of Dkk1 occurs at 13-14 days. ES cells incubated with Dkk1 differentiated into cardiomyocytes at 8-9 days (see FIG. 21). Thus, by comparison with ES cells not incubated with Dkk1, those incubated with Dkk1 showed early beating, an increased number of beating foci, an increased Nkx2.5 expression and increased contractile protein expression (see FIG. 21A). In addition, ES cells incubated with Dkk1 had elevated myosin light chain (MLC2a) mRNA, as measured by PCR, relative to ES cells not incubated with Dkk1 (see FIG. 21B).

### Example 6

### Heart-Inducing Activity of Dkk1 Resides Within the Carboxyl Fragment of Human Dkk1

This example demonstrates that the carboxyl terminal cysteine rich region (termed “C1”) of Dkk1 is a potent inducer of cardiac tissue from non-cardiac embryonic tissue.

A secreted version of the C-terminal fragment was made by fusing the secretory region of human Dkk1 to the C-terminal region cysteine rich region, as described above. This protein is referred to as “C1.” Briefly, the HindIII-Asp718 C-terminal fragment of human Dkk1 (SEQ ID NO: 1) was ligated to the sequence encoding the secretory signal of Dkk1 and cloned into a vector permitting in vitro transcription. As a control, full length Dkk1 was used. RNA was prepared in vitro from the vector as described above. The RNA encoded a polypeptide comprising amino acids 158-266 of SEQ ID NO: 2. The RNA was then injected into noncardiogenic frog embryonic mesoderm, as described above and in Schneider and Mercola (2001) Genes & Dev. 15:304. Expression of this protein induced hearts when expressed in noncardiogenic frog embryonic mesoderm. When compared to full length Dkk1, which is a heart inducer, C1 was between 10 and 100 times more potent.

In another example, C1 was expressed as a recombinant protein in host cells. Incubation of this protein with murine embryonic stem (ES) cells, as described above, also appeared to induce cardiomyocytes.

Based on the homology of members of the Dkk family of proteins in the cysteine rich domains, this discovery also predicts that the C-terminal cysteine-rich regions from structurally similar Dkk proteins (e.g., Dkk2) are also likely to be potent heart inducers.

### Example 7

### Effects of Recombinant Dkk1 on Cardiac Differentiation of ES Cells

Cells were grown on gelatin-coated plates without LIF for 2d, then allowed to aggregate on petri dish plastic for 2d, aggregates were then dispersed by mild trypsinization and plated onto gelatin-coated plates with rDkk1- or rC1-conditioned medium or control conditioned medium lacking rDkk for duration of experiment. Presence of beating foci was scored on days 8-14. Instances of beating foci presented as “beating”. Precocious beating sometimes occurred at day 8. The results for 8 trials are shown below.

| | | | | | | Positive |
|---|---|---|---|---|---|---|
| | | Beating Foci Detected in Dish | | | | effect |
| | | Dkk | | Controls | | of DKK |
| Trial | Dkk | Day | Day | Day | Day | relative |
| # | Type | 8 | 11-14 | 8 | 11-14 | to control |
| 1 | Dkk1 | beating | beating | no beating | beating | yes |
| 2 | C1 | beating | beating | no beating | beating | yes |
| 3 | Dkk1 | no beating | beating | no beating | beating | no |
| 4 | Dkk1 | no beating | beating | no beating | beating | no |
| 5 | Dkk1 | no beating | beating | no beating | beating | no |
| 6 | Dkk1 | no beating | beating | no beating | beating | no |
| 7 | Dkk1 | no beating | no beating | no beating | no beating | no |
| 8 | Dkk1 | no beating | beating | no beating | no beating | yes |

### Example 8

### Isolation of SP Cells from Quail Tissues

SP cells were isolated from quail by FACS isolation relying on Hoechst 33342 and propidium iodide to distinguish different cell populations. Cells were incubated in Hoechst 33342 at 37° C. for 60-90 minutes. Cells were then collected by centrifugation, washed in PBS and resuspended in a propidium iodide solution. As a negative control, a fraction of the primary cells were incubated in parallel with verapamil, which blocks Hoechst 33342 efflux. Sorting was performed on a FACS Advantage Plus flow cytometer and fluorescence of Hoechst 33342 and propidium iodide was measured on a linear scale (Goodell et al. (1996) J. Exp. Med. 183:1797; Goodell et al. (1997) Nat. Med. 3:1337 and Gussoni et al. (1999) Nature 401:390). The cells were then pelleted by centrifugation.

FIG. 22 shows FACS profiles of SP cells isolated. SP is the minor population within the boxed region and compreises 1.5%, 3.2%, 8.2% of bone marrow, skeletal muscle and cardiac muscle, respectively. MP stands for majority population. As indicated in FIG. 22, verapamil (right panels) blocks the channel(s) responsible for low dye retention causing SP cells to sort with the MP population.

A summary of the PCR data showing that SP cells express genetic markers indicative of tissue of origin, suggestive of cells biased or committed to a lineage within the SP population is set forth in Table 2:

TABLE 2

| Cells | Nkx2.5 | MyoD | myogenin |
|---|---|---|---|
| Heart SP | + | − | − |
| Heart MP | + | − | − |
| Sk. Musc. SP | − | + | + |
| Sk. Musc. MP | − | + | + |

Thus, cells in the heart SP population express Nkx2.5, which marks the heart-forming region in early embryos. In contrast, skeletal muscle SP cells do not express Nkx2.5. These results indicate that heart and skeletal muscle SP populations differ and raise the possibility that at least some heart SP cells might be predisposed or committed to a cardiac lineage.

### Equivalents

The present invention provides among other things novel methods and compositions for stimulating differentiation of stem cells into cardiac cells. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

### Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: US 2003/0013192A1; U.S. Pat. No. 6,159,462; U.S. Pat. No. 6,485,972; US 2002/0128439 A1; U.S. Pat. No. 6,133,232; US 2002/0061837; U.S. Pat. No. 6,033,906; U.S. Pat. No. 6,344,541; U.S. Pat. No. 6,200,806; US 2002/0142457; US 2002/0166134; U.S. Pat. No. 5,602,301; and US 2002/0160509.

We claim:

1. A method for stimulating differentiation of stem cells into cardiac cells, comprising contacting a population of cells comprising stem cells with a sufficient amount of at least one Wnt antagonist to stimulate differentiation of at least a portion of the stem cells into cardiac cells.

2. The method of claim 1, wherein the Wnt antagonist is an antagonist of one or more of the following: Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16.

3. The method of claim 2, wherein the Wnt antagonist is an antagonist of Wnt3a.

4. The method of claim 3, wherein the Wnt antagonist is an antagonist of Wnt8.

5. The method of claim 1, wherein the Wnt antagonist is one or more of the following:
polypeptides, nucleic acids, or small molecules.

6. The method of claim 5, wherein the antagonist is a polypeptide.

7. The method of claim 6, wherein the antagonist is one or more of the following polypeptides or a fragment thereof: a Dkk polypeptide, a crescent polypeptide, a cerberus polypeptide, an axin polypeptide, a Frzb polypeptide, a glycogen synthase kinase polypeptide, a T-cell factor polypeptide, or a dominant negative dishevelled polypeptide.

8. The method of claim 7, wherein the antagonist is a crescent polypeptide.

9. The method of claim 1, wherein the stem cells are embryonic stem (ES) cells.

10. The method of claim 1, wherein the stem cells are side population (SP) stem cells.

11. The method of claim 1, wherein the stem cells are germ cells.

12. The method of claim 1, wherein the stem cells are from a subject.

13. The method of claim 1, wherein the stem cells are vertebrate cells.

14. The method of claim 13, wherein stem cells are mammalian cells.

15. The method of claim 14, wherein stem cells are human cells.

16. The method of claim 1 which further comprises contacting the population of cells with at least one BMP polypeptide.

17. The method of claim 16, wherein the BMP is one or more of the following: BMP1, BMP2, BMP3, BMP4, BMP5, BMP6, BMP7, BMP8, BMP9, BMP10, BMP11, or BMP15.

18. The method of claim 17, wherein the BMP is BMP2 or BMP4.

19. The method of claim 16, wherein the BMP is human BMP.

20. A method for stimulating differentiation of stem cells into cardiac cells, comprising contacting a population of cells comprising stem cells with a Dkk protein or portion thereof sufficient to stimulate differentiation of a stem cell into a cardiac cell, such that the stem cells differentiate into cardiac cells.

21. The method of claim 20, wherein the Dkk protein is Dkk1 or Dkk2.

22. The method of claim 21, wherein the Dkk protein is human Dkk1 or human Dkk2.

23. The method of claim 22, wherein the Dkk protein comprises SEQ ID NO: 2 or 4.

24. The method of claim 20, wherein the Dkk protein is a fusion protein comprising an N-terminal cysteine rich domain of a Dkk1 protein and a C-terminal cysteine rich domain of a Dkk2 protein.

25. The method of claim 20, wherein the Dkk protein comprises the amino acid sequence set forth in SEQ ID NO: 5 or 6.

26. The method of claim 20, comprising contacting the population of cells with a fragment of a Dkk protein sufficient to stimulate differentiation of a stem cell into a cardiac cell.

27. The method of claim 26, wherein the fragment of the Dkk protein comprises at most about 110 amino acids and a C-terminal cysteine rich domain.

28. The method of claim 27, wherein the fragment of the Dkk protein comprises about amino acids 159 to 266 of SEQ ID NO: 2.

29. The method of claim 20, wherein the stem cells are embryonic stem (ES) cells.

30. The method of claim 20, wherein the stem cells are side population (SP) stem cells.

31. The method of claim 20, wherein the stem cells are germ cells.

32. The method of claim 20, wherein the stem cells are from a subject.

33. The method of claim 20, further comprising inhibiting LDL-receptor related protein (LRP) 6.

34. A method for producing cardiac cells from stem cells of a subject, comprising
obtaining stem cells from a subject; and
contacting the stem cells with a sufficient amount of at least one Wnt antagonist to stimulate the differentiation of the stem cells into cardiac cells, thereby producing cardiac cells.

35. The method of claim 34, wherein the Wnt antagonist is an antagonist of Wnt3a or Wnt 8.

36. The method of claim 34, wherein the Wnt antagonist is a Dkk1 or Dkk2 polypeptide.

37. The method of claim 34, wherein the Wnt antagonist is a human Dkk1 or Dkk2 polypeptide.

38. The method of claim 34, wherein the Wnt antagonist is a fragment of a Dkk polypeptide.

39. The method of claim 38, wherein the fragment of the Dkk protein comprises at most about 110 amino acids and a C-terminal cysteine rich domain.

40. The method of claim 38, wherein the fragment of the Dkk protein comprises about amino acids 159 to 266 of SEQ ID NO: 2.

41. The method of claim 38, wherein the Dkk protein comprises the amino acid sequence set forth in SEQ ID NO: 5 or 6.

42. The method of claim 34, wherein the Wnt antagonist is a crescent polypeptide.

43. The method of claim 34, wherein the stem cells are SP cells.

44. A composition, comprising:
an isolated population of cells comprising stem cells; and
a Wnt antagonist,
wherein the Wnt antagonist is in a sufficient concentration in the composition to cause more of the stem cells to differentiate into cardiac cells than would have differentiated in the absence of the Wnt antagonist.

45. The compositions of claim 44, further comprising a BMP protein.

46. A method for identifying a Wnt antagonist that has cardiomyogenesis inducing activity, comprising:
providing a population of cells comprising stem cells;
contacting the population of cells with one or more test compounds;
assaying for differentiation of the stem cells into cardiac cells; and
identifying a test compound that causes more of the stem cells to differentiate into cardiac cells than differentiated in the absence of the test compound, thereby identifying a Wnt antagonist with cardiomyogenesis inducing activity.

47. A method for stimulating differentiation of stem cells into cardiac cells, comprising contacting a population of cells comprising stem cells with a sufficient amount of at least one Wnt antagonist to stimulate differentiation of at least a portion of the stem cells into cardiac cells, wherein the Wnt antagonist was identified using the method of claim 46.

48. A method for inducing cardiomyogenesis in a vertebrate, comprising administering to the vertebrate a sufficient amount of at least one Wnt antagonist to stimulate differentiation of a stem cell into a cardiac cell, such that cardiomyogenesis is induced in the vertebrate.

49. A method for modulating lineage determination of a stem cell, comprising contacting a population of cells comprising stem cells with a sufficient amount of a Wnt antagonist to stimulate differentiation of the stem cells.

50. Isolated cardiac cells obtained according to the method of claim 1.

51. An isolated population of cardiac cells, wherein at least about 90% of the cells are cardiac cells.

52. A fragment of a Dkk protein that is at least about 5 times more potent than the full length Dkk protein in inducing differentiation of a stem cell into a cardiac cell.

53. A polypeptide comprising a fragment of a Dkk protein comprising at most about 110 amino acids and a C-terminal cysteine rich domain.

54. The polypeptide of claim 53, comprising about amino acids 159 to 266 of a Dkk1 protein.

55. The polypeptide of claim 54, comprising about amino acids 159 to 266 of SEQ ID NO: 2.

56. The polypeptide of claim 53, further comprising a signal peptide.

57. The polypeptide of claim 55, comprising a signal peptide consisting of about amino acids 1 to 31 of SEQ ID NO: 2.

58. An isolated nucleic acid encoding a polypeptide of claim 52.

59. An isolated nucleic acid encoding a polypeptide of claim 56.

60. A vector comprising the nucleic acid of claim 58.

61. A host cell comprising the nucleic acid of claim 58.
